(19) United States
(12) Patent Application Publication
(10) Pub. No.: US 2025/0277030 A1
(43) Pub. Date: Sep. 4, 2025

(54) ANTI-NAPI2B ANTIBODIES AND METHODS OF USE

(71) Applicant: Zymeworks BC Inc., Vancouver (CA)

(72) Inventors: Grant Raymond Wickman, Vancouver (CA); James R. Rich, Vancouver (CA); Andrea Hernandez Rojas, Vancouver (CA); Eric ESCOBAR-CABRERA, Burnaby (CA); Robert William Gene, Vancouver (CA); Stuart Daniel Barnscher, Vancouver (CA); Samuel Oliver Lawn, Vancouver (CA); Sukhbir Singh Kang, Vancouver (CA); Danny Chui, Vancouver (CA); Brandon Clavette, Vancouver (CA); Duncan Browman, Vancouver (CA); Gesa Volkers, Vancouver (CA)

(21) Appl. No.: 19/093,924

(22) Filed: Mar. 28, 2025

Related U.S. Application Data
(63) Continuation of application No. PCT/CA2023/051386, filed on Oct. 19, 2023.
(60) Provisional application No. 63/417,678, filed on Oct. 19, 2022.

(51) Int. Cl. C07K 16/28; A61K 39/00; A61K 47/68; A61P 35/00

(52) U.S. Cl. CPC C07K 16/28; A61K 47/68031; A61K 47/6849; A61P 35/00; A61K 2039/505; C07K 2317/24; C07K 2317/33; C07K 2317/34; C07K 2317/77; C07K 2317/92; C07K 2317/94

(57) ABSTRACT

Described herein are antibody constructs that bind human NaPi2b (or SLC34A2) and antibody-drug conjugates (ADCs) comprising an anti-NaPi2b antibody construct conjugated to a drug, such as a cytotoxin or an immune modulator, and their use as therapeutics or diagnostics, for example, in the treatment or diagnosis of cancer.

### CROSS-REFERENCE TO RELATED APPLICATIONS

This application is a continuation of PCT/CA2023/051386, filed Oct. 19, 2023, which claims priority to U.S. Application No. 63/417,678, filed Oct. 19, 2022, both of which are herein incorporated in their entirety by reference.

### FIELD

The present disclosure relates to the field of antibody therapeutics and, in particular, to antibodies targeting human sodium-dependent phosphate transporter 2B (hNaPi2b).

### SEQUENCE LISTING

The instant application contains a Sequence Listing which is hereby incorporated by reference in its entirety. Said XML copy, created on May 14, 2025, is named 2025 05 14 Amended ZYME093 Seq List.xml, and is 77,285 bytes in size.

### BACKGROUND

Sodium-dependent phosphate transporter 2B (NaPi2b) is a transmembrane protein encoded by the SLC34A2 gene. The NaPi2b polypeptide is 690 amino acids in length, with a limited extracellular domain of amino acids 188-361 exposed on the surface of cells. It is widely expressed in normal tissues and overexpressed in a variety of cancers including ovarian cancer, endometrial cancer, and lung cancer.

Given the overexpression of NaPi2b in certain types of cancers, NaPi2b-targeted agents have been studied in clinical trials for the treatment of cancer but have returned mixed results. A Phase I/II clinical trial to study upifitamab rilsodotin, an antibody-drug conjugate (ADC) of the NaPi2b-targeting antibody MX-35 with an auristatin-F payload (Dolaflexin platform) in patients with platinum-resistant ovarian cancer or non-small cell lung cancer (NSCLC) was undertaken by Mersana Therapeutics. The NSCLC arm of the study was discontinued due to lack of efficacy, while upifitamab rilsodotin was granted Fast Track Designation for the treatment of platinum-resistant ovarian cancer patients who have received three to four prior lines of therapy. Mersana has also completed a Phase I/II clinical trial of XMT-1592 in ovarian cancer; XMT-1592 is a site-specific ADC, comprised of antibody MX-35 conjugated to an auristatin-F payload using their Dolasynthen platform. However, the development of this ADC has been discontinued.

Lifastuzumab vedotin, an ADC of lifastuzumab with an MMAE payload was studied in a clinical trial sponsored by Genentech in patients with ovarian cancer or NSCLC, but this trial has since been discontinued.

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are anti-NaPi2b antibodies and methods of use. One aspect of the present disclosure relates to an antibody construct comprising an antigen-binding domain that binds to human NaPi2b (sodium-dependent phosphate transport protein 2B), the antigen-binding domain comprising: a heavy chain CDR1 (HCDR1) amino acid sequence comprising the sequence as set forth in SEQ ID NO: 7, a heavy chain CDR2 (HCDR2) amino acid sequence comprising the sequence as set forth in SEQ ID NO: 8, and a heavy chain CDR3 (HCDR3) amino acid sequence comprising the sequence as set forth in SEQ ID NO: 9, and a light chain CDR1 (LCDR1) amino acid sequence comprising the sequence as set forth in SEQ ID NO: 19, a light chain CDR2 (LCDR2) amino acid sequence comprising the sequence as set forth in SEQ ID NO: 20, and a light chain CDR3 (LCDR3) amino acid sequence comprising the sequence as set forth in SEQ ID NO: 18.

Another aspect of the present disclosure relates to a polynucleotide or set of polynucleotides encoding the anti-NaPi2b antibody construct as described herein.

Another aspect of the present disclosure relates to an expression vector or set of expression vectors comprising a polynucleotide or set of polynucleotides encoding the anti-NaPi2b antibody construct as described herein. Another aspect of the present disclosure relates to a host cell comprising the expression vector or set of expression vectors.

Another aspect of the present disclosure relates to an antibody-drug conjugate comprising the anti-NaPi2b antibody construct as described herein conjugated to one or more drug moieties.

Another aspect of the present disclosure relates to a pharmaceutical composition comprising an anti-NaPi2b antibody construct as described herein or an antibody-drug conjugate as described herein, and a pharmaceutically acceptable carrier or diluent.

Another aspect of the present disclosure relates to an anti-NaPi2b antibody construct as described herein or an antibody-drug conjugate as described herein for use in therapy, for example, in the treatment of cancer.

Another aspect of the present disclosure relates to a use of an anti-NaPi2b antibody construct as described herein or an antibody-drug conjugate as described herein in the manufacture of a medicament for the treatment of cancer.

Another aspect of the present disclosure relates to a method of inhibiting the growth of NaPi2b-positive tumor cells comprising contacting the cells with an anti-NaPi2b antibody construct as described herein or an antibody-drug conjugate as described herein.

Another aspect of the present disclosure relates to a method of treating a subject having a cancer comprising administering to the subject an effective amount of an anti-NaPi2b antibody construct as described herein or an antibody-drug conjugate as described herein.

### BRIEF DESCRIPTION OF THE DRAWINGS

FIG. 1A shows the sequence of the mouse heavy chain variable domain CDRs of the chimeric anti-NaPi2b antibody v23855 ported onto a human VH germline (IGHV1-46*03), and FIG. 1B shows the sequence of the mouse light chain variable domain CDRs of chimeric antibody v23855 ported onto a human VL framework (IGKVID-39*01). The CDRs were assigned with the AbM definition and marked in bold and underlined text.

FIG. 2A shows non-reducing (NR) SDS-PAGE profiles for all humanized variants and the parental chimeric variant 23855. FIG. 2B shows reducing (R) SDS-PAGE profiles for all humanized variants and parental chimeric variant 23855. FIG. 2C shows the UPLC-SEC profile for the parental mouse-human chimeric antibody v23855. FIG. 2D shows the UPLC-SEC profile for a representative humanized antibody, v29456.

FIG. 3A depicts binding of humanized antibody variant v29456, MX-35 (v18992) and lifastuzumab (v18993) to human NaPi2b. FIG. 3B depicts binding of humanized antibody variant v29456, MX-35 (v18992) and lifastuzumab (v18993) to cynomolgus NaPi2b. FIG. 3C depicts binding of humanized antibody variant v29456, MX-35 (v18992) and lifastuzumab (v18993) to mouse NaPi2b.

FIG. 4A depicts the N-curve analysis of binding to NaPi2b expressed on IGROV-1 cells for v29814. FIG. 4B depicts the N-curve analysis of binding to NaPi2b expressed on IGROV-1 cells for v36123. FIG. 4C depicts the N-curve analysis of binding to NaPi2b expressed on IGROV-1 cells for v36124. For each panel, the right curve shows the data for 500 pM constant binding partner and the left curve shows the data for 50 pM constant binding partner.

FIG. 5A shows a comparison of the ability of v23855 (parental chimeric), v29456 (H1L2), v18992 (MX35), and v18993 (lifastuzumab) to internalize in HCC-78 cells. FIG. 5B shows a comparison of the ability of v23855 (parental chimeric), v29456 (H1L2), v18992 (MX35), and v18993 (lifastuzumab) to internalize in NCI-H441 cells.

FIG. 6 depicts binding of parental chimeric antibody (v23855), humanized antibody variants v29452 and v29456, in addition to binding of MX35 and lifastuzumab ADCs to IGROV-1 cells.

FIG. 7A depicts the ability of humanized antibody variant v29456, and v29456 conjugated to DL1 or DL2 to bind to IGROV-1 cells. FIG. 7B depicts the ability of v29456, and v29456 conjugated to DL1 or DL2 to bind to HCC-78 cells.

FIG. 8A shows the cytotoxicity of ADCs of humanized antibody variant v29456 and ADCs of reference antibodies MX35 and lifastuzumab in OVCAR-3 cells. FIG. 8B shows the cytotoxicity of ADCs of v29456 and ADCs of reference antibodies MX35 and lifastuzumab in IGROV-1 cells. FIG. 8C shows the cytotoxicity of ADCs of v29456 and ADCs of reference antibodies MX35 and lifastuzumab in HCC-78 cells.

FIG. 9A depicts the in vivo efficacy of an ADC of the parental chimeric v23855 compared to an ADC of reference antibody v18992 (MX35) in an OVCAR-3 xenograft model of ovarian cancer. FIG. 9B depicts the in vivo efficacy of an ADC of the parental chimeric v23855 compared to an ADC of reference antibody v18993 (lifastuzumab) in an OVCAR-3 xenograft model of ovarian cancer.

FIG. 10 depicts the in vivo efficacy of an ADC of v29456 at 1, 3, and 10 mg/kg in an OVCAR-3 xenograft model of ovarian cancer.

FIG. 11A shows the in vivo efficacy of an ADC of v29456 at 1, 3, and 10 mg/kg in an NCI-H441 xenograft model of lung cancer. FIG. 11B shows the in vivo efficacy of an ADC of v29456 at 0.3, and 1 mg/kg in an NCI-H441 xenograft model of lung cancer.

FIG. 12A depicts the result of the Membrane Proteome Array™ assay using v38591. FIG. 12B depicts validation data for CLDN3, showing weak binding to CLDN3.

FIG. 13A-FIG. 13B depicts binding of humanized antibody variants v38591 and v29456 to IGROV-1 cells and to TOV-21G cells, compared to binding of lifastuzumab.

FIG. 14 shows the PK profile of v29456 and v18993 (lifastuzumab)-MC-VC-PABC-MMAE (DL3) in Tg32 mice.

### DETAILED DESCRIPTION

The present disclosure relates to antibody constructs that bind human sodium-dependent phosphate transporter 2B (NaPi2b). In certain embodiments, the anti-NaPi2b antibody constructs of the present disclosure are also capable of binding to cynomolgus monkey NaPi2b.

The present disclosure also relates to antibody-drug conjugates (ADCs) comprising an anti-NaPi2b antibody construct as described herein conjugated to a drug, such as a cytotoxin or an immune modulator. The anti-NaPi2b antibody constructs and ADCs of the present disclosure may find use, for example, as therapeutics or diagnostics. Certain aspects of the present disclosure relate to therapeutic methods and uses of the anti-NaPi2b antibody constructs and ADCs, for example, in the treatment of cancer. Some aspects relate to diagnostic methods and uses of the anti-NaPi2b antibody constructs and ADCs, for example, in the diagnosis or analysis of cancer.

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

A “complementarity determining region” or “CDR” is an amino acid sequence that contributes to antigen-binding specificity and affinity. “Framework” regions (FR) can aid in maintaining the proper conformation of the CDRs to promote binding between the antigen-binding region and an antigen. From N-terminus to C-terminus, both the light chain variable region (VL) and the heavy chain variable region (VH) of an antibody typically comprise the domains FR1, CDR1, FR2, CDR2, FR3, CDR3, and FR4. The three heavy chain CDRs are referred to herein as HCDR1, HCDR2, and HCDR3, and the three light chain CDRs are referred to as LCDR1, LCDR2, and LCDR3. CDRs provide the majority of contact residues for the binding of the antibody to the antigen or epitope. Often, the three heavy chain CDRs and the three light chain CDRs are required to bind antigen. However, in some instances, even a single variable domain can confer binding specificity to the antigen. Furthermore, as is known in the art, in some cases, antigen-binding may also occur through a combination of a minimum of one or more CDRs selected from the VH and/or VL domains, for example HCDR3.

A number of different definitions of the CDR sequences are in common use, including those described by Kabat et al. (1983, Sequences of Proteins of Immunological Interest, NIH Publication No. 369-847, Bethesda, MD), by Chothia et al. (1987, J Mol Biol, 196:901-917), as well as the IMGT, AbM (University of Bath) and Contact (MacCallum, et al., 1996, J Mol Biol, 262(5):732-745) definitions. By way of example, CDR definitions according to Kabat, Chothia, IMGT, AbM and Contact are provided in Table 1 below. Accordingly, as would be readily apparent to one skilled in the art, the exact numbering and placement of CDRs may differ based on the numbering system employed. However, it is to be understood that the disclosure herein of a VH includes the disclosure of the associated (inherent) heavy chain CDRs (HCDRs) as defined by any of the known numbering systems. Similarly, disclosure herein of a VL includes the disclosure of the associated (inherent) light chain CDRs (LCDRs) as defined by any of the known numbering systems.

TABLE 1
Common CDR Definitions¹

| | Heavy Chain | | | Light Chain | | |
|---|---|---|---|---|---|---|
| Definition | CDR1² | CDR2 | CDR3 | CDR1 | CDR2 | CDR3 |
| Kabat | H31-H35B | H50-H65 | H95-H102 | L24-L34 | L50-L56 | L89-L97 |
| Chothia | H26-H32, | H52-H56 | H95-H102 | L24-L34 | L50-L56 | L89-L97 |
| | H33 or H34 | | | | | |
| IMGT | H26-H33, | H51-H57 | H93-H102 | L27-L32 | L50-L52 | L89-L97 |
| | H34, H35, | | | | | |
| | H35A or | | | | | |
| | H35B | | | | | |
| AbM | H26-H35B | H50-H58 | H95-H102 | L24-L34 | L50-L56 | L89-L97 |
| Contact | H30-H35B | H47-H58 | H93-H101 | L30-L36 | L46-L55 | L89-L96 |

¹Either the Kabat or Chothia numbering system may be used for HCDR2, HCDR3 and the light chain CDRs for all definitions except Contact, which uses Chothia numbering.
²Using Kabat numbering. The position in the Kabat numbering scheme that demarcates the end of the Chothia and IMGT CDR-H1 loop varies depending on the length of the loop because Kabat places insertions outside of those CDR definitions at positions 35A and 35B. However, the IMGT and Chothia CDR-H1 loop can be unambiguously defined using Chothia numbering. CDR-H1 definitions using Chothia numbering: Kabat H31-H35, Chothia H26-H32, AbM H26-H35, IMGT H26-H33, Contact H30-H35.

The term “identical” in the context of two or more polynucleotide or polypeptide sequences, refers to two or more sequences or subsequences that are the same. Sequences are “substantially identical” if they have a percentage of amino acid residues or nucleotides that are the same (for example, about 80%, about 85%, about 90%, about 95%, or about 98% identity, over a specified region) when compared and aligned for maximum correspondence over a comparison window or over a designated region as measured using one of the commonly used sequence comparison algorithms as known to persons of ordinary skill in the art or by manual alignment and visual inspection. For sequence comparison, typically test sequences are compared to a designated reference sequence. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window” refers to a segment of a sequence comprising contiguous amino acid or nucleotide positions which may be, for example, from about 10 to 600 contiguous amino acid or nucleotide positions, or from about 10 to about 200, or from about 10 to about 150 contiguous amino acid or nucleotide positions over which a test sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, for example, by the local homology algorithm of Smith & Waterman, 1970, Adv. Appl. Math., 2:482c; by the homology alignment algorithm of Needleman & Wunsch, 1970, J. Mol. Biol., 48:443; by the search for similarity method of Pearson & Lipman, 1988, Proc. Natl. Acad. Sci. USA, 85:2444, or by computerized implementations of these algorithms (for example, GAP, BESTFJT, FASTA or TFASTA in the Wisconsin Genetics Software Package, Genetics Computer Group, Madison, WI), or by manual alignment and visual inspection (see, for example, Ausubel et al., Current Protocols in Molecular Biology, (1995 supplement), Cold Spring Harbor Laboratory Press). Examples of available algorithms suitable for determining percent sequence identity are the BLAST and BLAST 2.0 algorithms, which are described in Altschul et al., 1997, Nuc. Acids Res., 25:3389-3402, and Altschul et al., 1990, J. Mol. Biol., 215:403-410, respectively. Software for performing BLAST analyses is publicly available through the website for the National Center for Biotechnology Information (NCBI).

The term “subject,” as used herein, refers to an animal, in some embodiments a mammal, which is the object of treatment, observation or experiment. The animal may be a human, a non-human primate, a companion animal (for example, dog, cat, or the like), farm animal (for example, cow, sheep, pig, horse, or the like) or a laboratory animal (for example, rat, mouse, guinea pig, non-human primate, or the like). In certain embodiments, the subject is a human.

It is contemplated that any embodiment discussed herein can be implemented with respect to any method, use or composition disclosed herein, and vice versa.

Particular features, structures and/or characteristics described in connection with an embodiment disclosed herein may be combined with features, structures and/or characteristics described in connection with another embodiment disclosed herein in any suitable manner to provide one or more further embodiments.

It is also to be understood that the positive recitation of a feature in one embodiment, serves as a basis for excluding the feature in an alternative embodiment. For example, where a list of options is presented for a given embodiment or claim, it is to be understood that one or more option may be deleted from the list and the shortened list may form an alternative embodiment, whether or not such an alternative embodiment is specifically referred to.

### Anti-NaPi2b Antibody Constructs

The present disclosure relates to antibody constructs that specifically bind to human NaPi2b (hNaPi2b). In this context, the term “antibody construct” refers to a polypeptide or a set of polypeptides that comprises one or more antigen-binding domains, where each of the one or more antigen-binding domains specifically binds to an epitope or antigen. Where the antibody construct comprises two or more antigen-binding domains, each of the antigen-binding domains may bind the same epitope or antigen (i.e. the antibody construct is monospecific) or they may bind to different epitopes or antigens (i.e. the antibody construct is bispecific or multispecific). The antibody construct may further comprise a scaffold and the one or more antigen-binding domains can be fused or covalently attached to the scaffold, optionally via a linker.

In accordance with the present disclosure, the anti-NaPi2b antibody construct comprises at least one antigen-binding domain that specifically binds to hNaPi2b. By “specifically binds” to hNaPi2b, it is meant that the antibody construct binds to hNaPi2b but does not exhibit significant binding to any of human NaPi2a or NaPi2c. In certain embodiments, the anti-NaPi2b antibody constructs of the present disclosure may be capable of binding to a NaPi2b from one or more non-human species. In certain embodiments, the anti-NaPi2b antibody constructs of the present disclosure are capable of binding to cynomolgus monkey NaPi2b.

Human NaPi2b is also known as human “solute carrier family 34 member 2” or “SLC34A2.” The protein sequences of hNaPi2b from various sources are known in the art and readily available from publicly accessible databases, such as GenBank or UniProtKB. Examples of hNaPi2b sequences include for example those provided under NCBI reference numbers NP_006415.3, NP_001171470.2, and NP_001171469.2. An exemplary hNaPi2b protein sequence is provided in Table 2 as SEQ ID NO: 1 (UniProt ID: 095436). An exemplary cynomolgus monkey NaPi2b protein sequence is also provided in Table 2 (SEQ ID NO: 2; UniProt ID: A0A2K5UHY1), as is an exemplary mouse NaPi2b protein sequence (SEQ ID NO:3; UniProt ID: Q9DBP0).

TABLE 2
Human, Cynomolgus Monkey, and Mouse NaPi2b Protein Sequences

| | | SEQ |
|---|---|---|
| Organism | Sequence | ID NO |
| Human | MAPWPELGDAQPNPDKYLEGAAGQQPTAPDKSKETNKTDNTEAPV | 1 |
| | TKIELLPSYSTATLIDEPTEVDDPWNLPTLQDSGIKWSERDTKGKILC | |
| | FFQGIGRLILLLGFLYFFVCSLDILSSAFQLVGGKMAGQFFSNSSIMS | |
| | NPLLGLVIGVLVTVLVQSSSTSTSIVVSMVSSSLLTVRAAIPIIMGANI | |
| | GTSITNTIVALMQVGDRSEFRRAFAGATVHDFFNWLSVLVLLPVEV | |
| | ATHYLEIITQLIVESFHFKNGEDAPDLLKVITKPFTKLIVQLDKKVISQ | |
| | IAMNDEKAKNKSLVKIWCKTFTNKTQINVTVPSTANCTSPSLCWTD | |
| | GIQNWTMKNVTYKENIAKCQHIFVNFHLPDLAVGTILLILSLLVLCG | |
| | CLIMIVKILGSVLKGQVATVIKKTINTDFPFPFAWLTGYLAILVGAG | |
| | MTFIVQSSSVFTSALTPLIGIGVITIERAYPLTLGSNIGTTTTAILAALA | |
| | SPGNALRSSLQIALCHFFFNISGILLWYPIPFTRLPIRMAKGLGNISAK | |
| | YRWFAVFYLIIFFFLIPLTVFGLSLAGWRVLVGVGVPVVFIIILVLCLR | |
| | LLQSRCPRVLPKKLQNWNFLPLWMRSLKPWDAVVSKFTGCFQMR | |
| | CCCCCRVCCRACCLLCDCPKCCRCSKCCEDLEEAQEGQDVPVKAP | |
| | ETFDNITISREAQGEVPASDSKTECTAL | |
| Cynomolgus | MAPWPELGDAQPNPSKYLEGATSQQPITPDKSKETNKTDNTEVPVT | 2 |
| Monkey | KFELLPTYSTATLIEEPTEVDDPWNLPTLQDSGIKWSERDAKGKILC | |
| | VFQGIGRLILLLGFLYFFVCSLDVLSSAFQLVGGKMAGQFFSNSSIMS | |
| | NPLLGLVIGVLVTVFVQSSSTSTSIVVSMVASSLLTVRAAIPIIMGANI | |
| | GTSITNTIVALMQVGDRSEFRRAFAGATVHDFFNWLSVLVLLPVEV | |
| | ATHYLEVVTQLIVESFHFKNGEDAPDLLKVITKPFTKLIVQLDKKVIS | |
| | QIAMNDETAKNKSLVKIWCKTFTNMTQMNVTVPSMANCTSHSLC | |
| | WTDGIQIWTMKNVTYKENIAKCQHIFVNFHLPDLAVGIILLIISLLVL | |
| | CGCLIMIVKILGSVLKGQVATVIKKTINTDFPFPFAWLTGYLAILVGA | |
| | GMTFIVQSSSVFTSALTPLIGIGVITIERAYPLTLGSNIGTTTTAILAAL | |
| | ASPGNTLRSSLQIALCHFFFNISGILLWYPIPFTRLPIRMAKGLGNISA | |
| | KYRWFAVFYLIIFFFLIPLTVFGLSLAGWPVLVGVGVPVVFIIILVLCL | |
| | RLLQSRCPRVLPKKLQNWNFLPLCMHSLKPWDAVISKFTGPFQMPC | |
| | CCCCRVCCRVCCLLCGCPKCCRCSKCCQDLEEGQEAQGVPVKAPE | |
| | TFDNITISREAQGEVRAPDSKTECTAL | |
| Mouse | MAPWPELENAQPNPGKFIEGASGPQSSIPAKDKEASKTNDNGTPVA | 3 |
| | KTELLPSYSALVLIEEHPEGTDPWDLPELQDTGIKWSERDTKGKTLC | |
| | IFQGVGKFILLLGFLYLFVCSLDVLSSAFQLVGGKVAGQFFSNNSIM | |
| | SNPVAGLVIGVLVTVMVQSSSTSSSIIVSMVASSLLTVRAAIPIIMGA | |
| | NIGTSITNTIVALMQAGDRNEFRRAFAGATVHDFFNWLSVFVLLPLE | |
| | AATHYLEILTNLVLETFKFQNGEDAPDILKVITDPFTKLIIQLDKKVI | |
| | QQIAMGDSAAQNKSLIKIWCKSITNVTEMNVTVPSTDNCTSPSYCW | |
| | TDGIQTWTIQNVTQKENIAKCQHIFVNFSLPDLAVGIILLTVSLVVLC | |
| | GCLIMIVKLLGSVLRGQVATVIKKTLNTDFPFPFAWLTGYLAILVGA | |
| | GMTFIVQSSSVFTSAMTPLIGIGVISIERAYPLTLGSNIGTTTTAILAAL | |
| | ASPGNTLRSSLQIALCHFFFNISGILLWYPIPFTRLPIRLAKGLGNISAK | |
| | YRWFAVFYLIFFFFVTPLTVFGLSLAGWPVLVGVGVPIILLLLLVLCL | |
| | RMLQFRCPRILPLKLRDWNFLPLWMHSLKPWDNVISLATTCFQRRC | |
| | CCCCRVCCRVCCMVCGCKCCRCSKCCRDQGEEEEEKEQDIPVKAS | |
| | GAFDNAAMSKECQDEGKGQVEVLSMKALSNTTVF | |

Specific binding of an antigen-binding domain to a target antigen or epitope may be measured, for example, through an enzyme-linked immunosorbent assay (ELISA), a surface plasmon resonance (SPR) technique (employing, for example, a BIAcore instrument) (Liljeblad et al., 2000, Glyco J, 17:323-329), flow cytometry or a traditional binding assay (Heeley, 2002, Endocr Res, 28:217-229). In certain embodiments, specific binding may be defined as the extent of binding to a non-target protein (such as hNaPi2a or hNaPi2c) being less than about 5% to 10% of the binding to hNaPi2b as measured by ELISA or flow cytometry, for example.

The term “dissociation constant (K_{D} or K_{d})” as used herein, is intended to refer to the equilibrium dissociation constant of a particular ligand-protein interaction. As used herein, ligand-protein interactions refer to, but are not limited to protein-protein interactions or antibody-antigen interactions. The K_{D} measures the propensity of two proteins complexed together (e.g. AB) to dissociate reversibly into constituent components (A+B), and is defined as the ratio of the rate constant of dissociation, also called the “off-rate (k_{off})”, to the association rate constant, or “on-rate (kₒₙ)”. Thus, K_{D} equals k_{off}/kₒₙ and is expressed as a molar concentration (M). It follows that the smaller the K_{D}, the stronger the affinity of binding, and thus a decrease in K_{D} indicates an increase in affinity. Therefore, a K_{D} of 1 mM indicates weak binding affinity compared to a K_{D} of 1 nM. Affinity is sometimes measured in terms of a K_{A} or Kₐ, which is the reciprocal of the K_{D} or K_{d}. K_{D} between antibody and its antigen can be determined using methods well established in the art. One method for determining such K_{D} is by using surface plasmon resonance (SPR), typically using a biosensor system such as a Biacore® system. Isothermal titration calorimetry (ITC) is another method that can be used to measure K_{D}. The Octet™ system may also be used to measure the affinity of antibodies for a target antigen.

In certain embodiments, specific binding of an antibody construct for NaPi2b may be defined by a dissociation constant (K_{d} or K_{D}) of <1 μM, for example, <500 nM, <250 nM, <100 nM, <50 nM, or <10 nM. In certain embodiments, specific binding of an antibody construct for a particular antigen or an epitope may be defined by a dissociation constant (K_{D}) of 10⁻⁶ M or less, for example, 10⁻⁷ M or less, or 10⁻⁸ M or less. In some embodiments, specific binding of an antibody construct for a particular antigen or an epitope may be defined by a dissociation constant (K_{D}) between 10⁻⁶ M and 10⁻⁹ M, for example, between 10⁻⁷ M and 10⁻⁹ M. As is known in the art the numerical value of the dissociation constant obtained may vary depending on how it is tested. For example, the expression level of NaPi2b in the cell line, format of the antibody construct (i.e. monovalent or bivalent), and type of assay (i.e. ELISA or flow cytometry), may affect the numerical value of the dissociation constant when measured in a cell-based assay. The data provided in the Examples illustrate this general point.

In some embodiments, the anti-NaPi2b antibody constructs of the present disclosure have a Kd that is lower than that of reference antibody lifastuzumab, and comparable to that of reference antibody MX35, when measured by flow cytometry in cells that express NaPi2b at high levels. Accordingly, in these embodiments, the anti-NaPi2b antibody constructs of the present disclosure comprise an antigen-binding domain having an affinity for human NaPi2b that is greater than that of reference antibody lifastuzumab and comparable to that of reference antibody MX35.

In certain embodiments the anti-NaPi2b antibody constructs exhibit comparable levels of internalization to the reference antibody MX35 and exhibit greater levels of internalization compared to reference antibody lifastuzumab in high and mid NaPi2b-expressing cells. In some embodiments, internalization is measured after 4 hours, after 5 hours or after 24 hours of treatment.

Antibody internalization may be measured using art-known methods, for example, by a direct internalization method according to the protocol detailed in Schmidt, M. et al., 2008, Cancer Immunol. Immunother., 57:1879-1890, or using commercially available fluorescent dyes such as the pHAb Dyes (Promega Corporation, Madison, WI), pHrodo iFL and Deep Red Dyes (ThermoFisher Scientific Corporation, Wahban, MA) and Incucyte® Fabfluor-pH Antibody Labeling Reagent (Sartorius AG, Göttingen, Germany) and analysis techniques such as microscopy, FACS, high content imaging or other plate-based assays.

NaPi2b expression varies depending on cell type as indicated throughout the disclosure and the level of NaPi2b expression is referred to herein as “high”, “mid,” “low” or “negative.” These terms are used for reference to describe levels of expression in general according to the designations shown in Table 10.1 in Example 10 and are not intended to be limited to the specific numerical values for average NaPi2b protein per cell included therein.

### Antigen-Binding Domains

The anti-NaPi2b antibody constructs of the present disclosure comprise at least one antigen-binding domain that is capable of binding to hNaPi2b. The at least one antigen-binding domain capable of binding to hNaPi2b typically is an immunoglobulin-based binding domain, such as an antigen-binding antibody fragment. Examples of an antigen-binding antibody fragment include, but are not limited to, a Fab fragment, a Fab′ fragment, a single chain Fab (scFab), a single chain Fv (scFv) and a single domain antibody (sdAb).

A “Fab fragment” contains the constant domain of the light chain (CL) and the first constant domain of the heavy chain (CH1) along with the variable domains of the light and heavy chains (VL and VH, respectively). Fab′ fragments differ from Fab fragments by the addition of a few amino acid residues at the C-terminus of the heavy chain CH1 domain, including one or more cysteines from the antibody hinge region. A Fab fragment may also be a single-chain Fab molecule, i.e. a Fab molecule in which the Fab light chain and the Fab heavy chain are connected by a peptide linker to form a single peptide chain. For example, the C-terminus of the Fab light chain may be connected to the N-terminus of the Fab heavy chain in the single-chain Fab molecule.

An “scFv” includes a heavy chain variable domain (VH) and alight chain variable domain (VL) of an antibody in a single polypeptide chain. The scFv may optionally further comprise a polypeptide linker between the VH and VL domains which enables the scFv to form a desired structure for antigen binding. For example, an scFv may include a VL connected from its C-terminus to the N-terminus of a VH by a polypeptide linker. Alternately, an scFv may comprise a VH connected through its C-terminus to the N-terminus of a VL by a polypeptide linker (see review in Pluckthun in The Pharmacology of Monoclonal Antibodies, vol. 113, Rosenburg and Moore eds., Springer-Verlag, New York, pp. 269-315 (1994)).

An “sdAb” format refers to a single immunoglobulin domain. The sdAb may be, for example, of camelid origin. Camelid antibodies lack light chains and their antigen-binding sites consist of a single domain, termed a “VHH.” An sdAb comprises three CDR/hypervariable loops that form the antigen-binding site: CDR1, CDR2 and CDR3. sdAbs are fairly stable and easy to express, for example, as a fusion with the Fc chain of an antibody (see, for example, Harmsen & De Haard, 2007, Appl. Microbiol Biotechnol., 77(1):13-22).

In those embodiments in which the anti-NaPi2b antibody constructs comprise two or more antigen-binding domains, each additional antigen-binding domain may independently be an immunoglobulin-based domain, such as an antigen-binding antibody fragment, or a non-immunoglobulin-based domain, such as a non-immunoglobulin-based antibody mimetic, or other polypeptide or small molecule capable of specifically binding to its target, for example, a natural or engineered ligand. Non-immunoglobulin-based antibody mimetic formats include, for example, anticalins, fynomers, affimers, alphabodies, DARPins and avimers.

The present disclosure describes herein the identification of a mouse antibody that specifically binds hNaPi2b; a mouse-human chimeric variant of this antibody is identified as variant 23855. The anti-NaPi2b antibody construct of the present disclosure comprises an antigen-binding domain derived from this mouse antibody or humanized antibody variants of same. Representative humanized antibody variants (v29449, v29450, v29451, v29452, v29453, v29454, v29455, v29456, v29457, v29458, v29459, and v29460) of the mouse antibody are also described. In certain embodiments, the anti-NaPi2b antibody constructs described herein specifically bind human NaPi2b having the sequence as set forth in SEQ ID NO:1.

In certain embodiments, the anti-NaPi2b antibody construct competes with any one of humanized antibody variants v29449, v29450, v29451, v29452, v29453, v29454, v29455, v29456, v29457, v29458, v29459, and v29460, or with parental chimeric antibody v23855, for binding to human NaPi2b. In assessing competition as described below, each of variants v29449, v29450, v29451, v29452, v29453, v29454, v29455, v29456, v29457, v29458, v29459, v29460, and v23855, are referred to as a competition reference antibody.

One can determine whether antibody constructs compete with variants v29449, v29450, v29451, v29452, v29453, v29454, v29455, v29456, v29457, v29458, v29459, and v29460, or parental chimeric antibody v23855 for binding to hNaPi2b using competition assays known in the art. For example, the competition reference antibody is first allowed to bind to hNaPi2b under saturating conditions and then the ability of the test antibody construct to bind to hNaPi2b is measured. If the test antibody construct is able to bind to hNaPi2b at the same time as the competition reference antibody, then the test antibody construct is considered to bind to a different epitope than the competition reference antibody. Conversely, if the test antibody construct is not able to bind to hNaPi2b at the same time as the competition reference antibody, then the test antibody construct is considered to bind to the same epitope, to an overlapping epitope, or to an epitope that is in close proximity to the epitope bound by the competition reference antibody. Such competition assays can be performed using techniques such as ELISA, radioimmunoassay, surface plasmon resonance (SPR), bio-layer interferometry, flow cytometry and the like. An “antibody that competes with” a competition reference antibody refers to an antibody that blocks binding of the reference antibody to its epitope in a competition assay by 50% or more.

In certain embodiments, the anti-NaPi2b antibody constructs of the present disclosure comprise at least one antigen-binding domain that specifically binds to hNaPi2b, where the antigen-binding domain comprises a set of CDRs based on the CDRs of parental chimeric antibody v23855 described herein. The CDR sequences of the parental chimeric antibody v23855 and representative humanized antibody variants are shown in Table 3.

TABLE 3
CDR sequences of anti-NaPi2b antibody constructs

| Numbering | | | | | | |
|---|---|---|---|---|---|---|
| System | Sequence | | | | | |
| | | SEQ | | SEQ | | SEQ |
| | Heavy Chain | ID | Heavy Chain | ID | Heavy Chain | ID |
| | CDR1 | NO | CDR2 | NO | CDR3 | NO |
| IMGT | GFTFTSYN | 4 | ISPGNGDL | 5 | ARGRTGMGAVDY | 6 |
| Kabat | SYNVH | 7 | AISPGNGDLS | 8 | GRTGMGAVDY | 9 |
| | | | YNQRFKD | | | |
| Chothia | GFTFTSY | 10 | SPGNGD | 11 | GRTGMGAVDY | 9 |
| AbM | GFTFTSYNVH | 12 | AISPGNGDLS | 13 | GRTGMGAVDY | 9 |
| Contact | TSYNVH | 14 | WIAAISPGNG | 15 | ARGRTGMGAVD | 16 |
| | | | DLS | | | |
| | | SEQ | | SEQ | | SEQ |
| | Light Chain | ID | Light Chain | ID | Light Chain | ID |
| | CDR1 | NO | CDR2 | NO | CDR3 | NO |
| IMGT | QDVYSY | 17 | YTS | — | QQYNIFPWT | 18 |
| Kabat | RASQDVYSYLN | 19 | YTSRLQS | 20 | QQYNIFPWT | 18 |
| Chothia | RASQDVYSYLN | 19 | YTSRLQS | 20 | QQYNIFPWT | 18 |
| AbM | RASQDVYSYLN | 19 | YTSRLQS | 20 | QQYNIFPWT | 18 |
| Contact | YSYLNWY | 21 | LLIYYTSRLQ | 22 | QQYNIFPW | 23 |

In certain embodiments, the anti-NaPi2b antibody constructs of the present disclosure comprise an antigen-binding domain having heavy chain CDR amino acid sequences (HCDR1, HCDR2 and HCDR3) comprising the sequences as set forth in SEQ ID NOs: 7, 8, and 9, and light chain CDR amino acid sequences (LCDR1, LCDR2 and LCDR3) comprising the sequences as set forth in SEQ ID NOs: 19, 20, and 18.

In certain embodiments, the anti-NaPi2b antibody constructs of the present disclosure comprise an antigen-binding domain having:
(i) an HCDR1 amino acid sequence selected from the HCDR1 amino acid sequences of any one of variants v23855, v29449, v29450, v29451, v29452, v29453, v29454, v29455, v29456, v29457, v29458, v29459, or v29460; an HCDR2 amino acid sequence selected from the HCDR2 amino acid sequences of any one of variants v23855, v29449, v29450, v29451, v29452, v29453, v29454, v29455, v29456, v29457, v29458, v29459, or v29460, and an HCDR3 amino acid sequence selected from the HCDR3 amino acid sequences of any one of variants v23855, v29449, v29450, v29451, v29452, v29453, v29454, v29455, v29456, v29457, v29458, v29459, or v29460, and
(ii) an LCDR1 amino acid sequence selected from the LCDR1 amino acid sequences of any one of variants v23855, v29449, v29450, v29451, v29452, v29453, v29454, v29455, v29456, v29457, v29458, v29459, or v29460; an LCDR2 amino acid sequence selected from the LCDR2 amino acid sequences of any one of variants v23855, v29449, v29450, v29451, v29452, v29453, v29454, v29455, v29456, v29457, v29458, v29459, or v29460, and an LCDR3 amino acid sequence selected from the LCDR3 amino acid sequences of any one of variants v23855, v29449, v29450, v29451, v29452, v29453, v29454, v29455, v29456, v29457, v29458, v29459, or v29460, wherein the CDR amino acid sequences are as defined by any one of the IMGT, Chothia, Kabat, Contact or AbM numbering systems (see Table 3).

In certain embodiments, the anti-NaPi2b antibody constructs of the present disclosure comprise an antigen-binding domain having heavy chain CDR amino acid sequences (HCDR1, HCDR2 and HCDR3) selected from the heavy chain CDR amino acid sequences of any one of variants v23855, v29449, v29450, v29451, v29452, v29453, v29454, v29455, v29456, v29457, v29458, v29459, or v29460, as defined by any one of the IMGT, Chothia, Kabat, Contact or AbM numbering systems, and light chain CDR amino acid sequences (LCDR1, LCDR2 and LCDR3) selected from the light chain CDR amino acid sequences of any one of variants v23855, v29449, v29450, v29451, v29452, v29453, v29454, v29455, v29456, v29457, v29458, v29459, or v29460, as defined by any one of the IMGT, Chothia, Kabat, Contact or AbM numbering systems.

In certain embodiments, the anti-NaPi2b antibody constructs of the present disclosure comprise an antigen-binding domain comprising heavy chain CDR amino acid sequences (HCDR1, HCDR2 and HCDR3) and light chain CDR amino acid sequences (LCDR1, LCDR2 and LCDR3) of any one of variants v23855, v29449, v29450, v29451, v29452, v29453, v29454, v29455, v29456, v29457, v29458, v29459, or v29460, as defined by any one of the IMGT, Chothia, Kabat, Contact or AbM numbering systems.

In certain embodiments, the anti-NaPi2b antibody constructs of the present disclosure comprise an antigen-binding domain having a VH sequence comprising the CDR sequences of the VH sequence of any one of variants v23855, v29449, v29450, v29451, v29452, v29453, v29454, v29455, v29456, v29457, v29458, v29459, or v29460. In certain embodiments, the anti-NaPi2b antibody constructs of the present disclosure comprise an antigen-binding domain having a VL sequence comprising the CDR sequences of the VL sequence of any one of variants v23855, v29449, v29450, v29451, v29452, v29453, v29454, v29455, v29456, v29457, v29458, v29459, or v29460.

One skilled in the art will appreciate that a limited number of amino acid substitutions may be introduced into the CDR sequences or into the VH or VL sequences of known antibodies without the antibody losing its ability to bind its target. Candidate amino acid substitutions may be identified by computer modeling or by art-known techniques such as alanine scanning, with the resulting variants being tested for binding activity by standard techniques. Accordingly, in certain embodiments, the anti-NaPi2b antibody constructs of the present disclosure comprise an antigen-binding domain that comprises a set of CDRs (i.e. heavy chain HCDR1, HCDR2 and HCDR3, and light chain LCDR1, LCDR2 and LCDR3) that have 90% or greater, 95% or greater, 98% or greater, 99% or greater, or 100% sequence identity to a set of CDRs of any one of variants v23855, v29449, v29450, v29451, v29452, v29453, v29454, v29455, v29456, v29457, v29458, v29459, or v29460, where the % sequence identity is calculated across all six CDRs and where the antigen-binding domain retains the ability to bind hNaPi2b.

In certain embodiments, the anti-NaPi2b antibody constructs of the present disclosure comprise an antigen-binding domain that comprises a variant of the set of CDR sequences of any one of variants v23855, v29449, v29450, v29451, v29452, v29453, v29454, v29455, v29456, v29457, v29458, v29459, or v29460, where the variant comprises between 1 and 10 amino acid substitutions across the set of CDRs (i.e. the CDRs may be modified by up to 10 amino acid substitutions with any combination of the six CDRs being modified), and where the antigen-binding domain retains the ability to bind hNaPi2b. In some embodiments, the anti-NaPi2b antibody constructs of the present disclosure comprise an antigen-binding domain that comprises a variant of the set of CDR sequences of any one of variants v23855, v29449, v29450, v29451, v29452, v29453, v29454, v29455, v29456, v29457, v29458, v29459, or v29460, where the variant comprises between 1 and 7 amino acid substitutions, between 1 and 5 amino acid substitutions, between 1 and 4 amino acid substitutions, between 1 and 3 amino acid substitutions, between 1 and 2 amino acid substitutions, or 1 amino acid substitution, across the set of CDRs, and where the antigen-binding domain retains the ability to bind hNaPi2b.

In certain embodiments, the anti-NaPi2b antibody constructs of the present disclosure comprise an antigen-binding domain that comprises a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH sequence of any one of variants v23855, v29449, v29450, v29451, v29452, v29453, v29454, v29455, v29456, v29457, v29458, v29459, or v29460, where the antigen-binding domain retains the ability to bind hNaPi2b. In certain embodiments, the anti-NaPi2b antibody constructs of the present disclosure comprise an antigen-binding domain that comprises a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL sequence of any one of variants v23855, v29449, v29450, v29451, v29452, v29453, v29454, v29455, v29456, v29457, v29458, v29459, or v29460, where the antigen-binding domain retains the ability to bind hNaPi2b.

In certain embodiments, the anti-NaPi2b antibody constructs of the present disclosure comprise an antigen-binding domain comprising a VH amino acid sequence selected from the VH amino acid sequences of any one of variants v23855, v29449, v29450, v29451, v29452, v29453, v29454, v29455, v29456, v29457, v29458, v29459, or v29460. In certain embodiments, the anti-NaPi2b antibody constructs of the present disclosure comprise an antigen-binding domain comprising a VL amino acid sequence selected from the VL amino acid sequences of any one of variants v23855, v29449, v29450, v29451, v29452, v29453, v29454, v29455, v29456, v29457, v29458, v29459, or v29460.

In certain embodiments, the anti-NaPi2b antibody constructs of the present disclosure comprise an antigen-binding domain that comprises a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH sequence of v23855, and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL sequence of v23855, where the antigen-binding domain retains the ability to bind hNaPi2b.

In certain embodiments, the anti-NaPi2b antibody constructs of the present disclosure comprise an antigen-binding domain that comprises a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH sequence of v29456, and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL sequence of v29456, where the antigen-binding domain retains the ability to bind hNaPi2b.

In certain embodiments, the anti-NaPi2b antibody constructs of the present disclosure comprise an antigen-binding domain that comprises a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH sequence of v29452, and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL sequence of v29452, where the antigen-binding domain retains the ability to bind hNaPi2b.

In some embodiments, the anti-NaPi2b antibody constructs of the present disclosure comprise the VH and VL sequences of any one of v23855, v29449, v29450, v29451, v29452, v29453, v29454, v29455, v29456, v29457, v29458, v29459, or v29460. The SEQ ID NOs: of the VH and VL sequences of these variants are provided below in Table 4. The sequences themselves are provided in Table 2.4 of the Examples.

TABLE 4
VH and VL sequences of parental chimeric
and humanized anti-NaPi2b antibodies

| | VH sequence | VL sequence |
|---|---|---|
| Variant | (SEQ ID NO:) | (SEQ ID NO:) |
| 23855 | 31 | 32 |
| 29449 | 27 | 30 |
| 29450 | 26 | 30 |
| 29451 | 25 | 30 |
| 29452 | 24 | 30 |
| 29453 | 27 | 29 |
| 29454 | 26 | 29 |
| 29455 | 25 | 29 |
| 29456 | 24 | 29 |
| 29457 | 27 | 28 |
| 29458 | 26 | 28 |
| 29459 | 25 | 28 |
| 29460 | 24 | 28 |

In some embodiments, the anti-NaPi2b antibody construct of the present disclosure comprises the VH sequence and the VL sequence of v29456. In some embodiments, the anti-NaPi2b antibody construct of the ADC of the present disclosure comprises the VH sequence and the VL sequence of v29452.

In certain embodiments, the anti-NaPi2b antibody construct of the present disclosure comprise a) a VH sequence having the 3 HCDRs of v29456 and having at least at least 91%, at least 92%, at least 93%, at least 94%, at least 95%, at least 96%, at least 97%, at least 98%, or at least 99% identity to the VH sequence of v29456, and b) a VL sequence having the 3 LCDRs of v29456 and having at least at least 91%, at least 92%, at least 93%, at least 94%, at least 95%, at least 96%, at least 97%, at least 98%, or at least 99% identity to the VL sequence of v29456, wherein the HCDRs and LCDRs are defined by any one of the IMGT, Chothia, Kabat, Contact or AbM numbering systems.

In certain other embodiments, the anti-NaPi2b antibody construct of the present disclosure comprise a) a VH sequence having the 3 HCDRs of v29452 and having at least at least 91%, at least 92%, at least 93%, at least 94%, at least 95%, at least 96%, at least 97%, at least 98%, or at least 99% identity to the VH sequence of v29452, and b) a VL sequence having the 3 LCDRs of v29452 and having at least at least 91%, at least 92%, at least 93%, at least 94%, at least 95%, at least 96%, at least 97%, at least 98%, or at least 99% identity to the VL sequence of v29452, wherein the HCDRs and LCDRs are defined by any one of the IMGT, Chothia, Kabat, Contact or AbM numbering systems.

### Formats

The anti-NaPi2b antibody constructs of the present disclosure may have various formats. The minimal component of the anti-NaPi2b antibody construct is an antigen-binding domain that binds to hNaPi2b. The anti-NaPi2b antibody constructs may further optionally comprise one or more additional antigen-binding domains and/or a scaffold. In those embodiments in which the anti-NaPi2b antibody construct comprises two or more antigen-binding domains, each additional antigen-binding domain may bind to the same epitope within hNaPi2b, may bind to a different epitope within hNaPi2b, or may bind to a different antigen. Thus, the anti-NaPi2b antibody construct may be, for example, monospecific, biparatopic, bispecific or multispecific.

In certain embodiments, the anti-NaPi2b antibody construct comprises at least one antigen-binding domain that binds to hNaPi2b and a scaffold, where the antigen-binding domain is operably linked to the scaffold. The term “operably linked,” as used herein, means that the components described are in a relationship permitting them to function in their intended manner. Suitable scaffolds are described below.

In certain embodiments, the anti-NaPi2b antibody construct comprises two antigen-binding domains optionally operably linked to a scaffold. In some embodiments, the anti-NaPi2b antibody construct may comprise three or four antigen-binding domains and optionally a scaffold. In these formats, when comprising a scaffold, at least a first antigen-binding domain is operably linked to the scaffold and the remaining antigen-binding domain(s) may each independently be operably linked to the scaffold or to the first antigen-binding domain or, when more than two antigen-binding domains are present, to another antigen-binding domain.

Anti-NaPi2b antibody constructs that lack a scaffold may comprise a single antigen-binding domain in an appropriate format, such as an sdAb, or they may comprise two or more antigen-binding domains optionally operably linked by one or more linkers. In such anti-NaPi2b antibody constructs, the antigen-binding domains may be in the form of scFvs, Fabs, sdAbs, or a combination thereof. For example, using scFvs as the antigen-binding domains, formats such as a tandem scFv ((scFv)₂ or taFv) may be constructed, in which the scFvs are connected together by a flexible linker. scFvs may also be used to construct diabody formats, which comprise two scFvs connected by a short linker (usually about 5 amino acids in length). The restricted length of the linker results in dimerization of the scFvs in a head-to-tail manner. In any of the preceding formats, the scFvs may be further stabilized by inclusion of an interdomain disulfide bond. For example, a disulfide bond may be introduced between VL and VH through substitution of non-cysteine residues to cysteine residues in each chain (for example, at position 44 in VH and 100 in VL) (see, for example, Fitzgerald et al., 1997, Protein Engineering, 10:1221-1225), or a disulfide bond may be introduced between two VHs to provide a construct having a DART format (see, for example, Johnson et al., 2010, J Mol. Biol., 399:436-449).

Similarly, formats comprising two sdAbs, such as VHs or VHHs, connected together through a suitable linker may be employed in some embodiments. Other examples of anti-NaPi2b antibody construct formats that lack a scaffold include those based on Fab fragments, for example, Fab₂ and F(ab′)₂ formats, in which the Fab fragments are connected through a linker or an IgG hinge region.

Combinations of antigen-binding domains in different forms may also be employed to generate alternative scaffold-less formats. For example, an scFv or a sdAb may be fused to the C-terminus of either or both of the light and heavy chain of a Fab fragment resulting in a bivalent (Fab-scFv/sdAb) construct.

In certain embodiments, the anti-NaPi2b antibody construct may be in an antibody format that is based on an immunoglobulin (Ig). This type of format is referred to herein as a full-size antibody format (FSA) or Mab format and includes anti-NaPi2b antibody constructs that comprise two Ig heavy chains and two Ig light chains. In certain embodiments, the anti-NaPi2b antibody construct may be based on an IgG class immunoglobulin, for example, an IgG1, IgG2, IgG3 or IgG4 immunoglobulin. In some embodiments, the anti-NaPi2b antibody construct may be based on an IgG1 immunoglobulin. In the context of the present disclosure, when an anti-NaPi2b antibody construct is based on a specified immunoglobulin isotype, it is meant that the anti-NaPi2b antibody construct comprises all or a portion of the constant region of the specified immunoglobulin isotype. For example, an anti-NaPi2b antibody construct based on a given Ig isotype may comprise at least one antigen-binding domain operably linked to an Ig scaffold, where the scaffold comprises an Fc region from the given isotype and optionally an Ig hinge region from the same or a different isotype. It is to be understood that the anti-NaPi2b antibody constructs may also comprise hybrids of isotypes and/or subclasses in some embodiments. It is also to be understood that the Fc region and/or hinge region may optionally be modified to impart one or more desirable functional properties as is known in the art. Thus, in certain embodiments, the anti-NaPi2b antibody construct comprises a VH amino acid sequence fused to IgG1 constant domain amino acid sequences (i.e. CH1, hinge, CH2, CH3 amino acid sequences) and a VL amino acid sequence fused to kappa or lambda constant amino acid sequences domain (i.e. CL amino acid sequences). Exemplary amino acid sequences are provided in the Examples and Sequence Tables.

In some embodiments, the anti-NaPi2b antibody constructs may be derived from two or more immunoglobulins that are from different species, for example, the anti-NaPi2b antibody construct may be a chimeric antibody or a humanized antibody. The terms “chimeric antibody” and “humanized antibody” both refer generally to antibodies that combine immunoglobulin regions or domains from more than one species.

A “chimeric antibody” typically comprises at least one variable domain from a non-human antibody, such as a rabbit or rodent (for example, murine) antibody, and at least one constant domain from a human antibody. The human constant domain of a chimeric antibody need not be of the same isotype as the non-human constant domain it replaces. Chimeric antibodies are discussed, for example, in Morrison et al., 1984, Proc. Natl. Acad. Sci. USA, 81:6851-55, and U.S. Pat. No. 4,816,567.

A “humanized antibody” is a type of chimeric antibody that contains minimal sequence derived from a non-human antibody. Generally, humanized antibodies are human immunoglobulins (recipient antibody) in which residues from a hypervariable region of the recipient are replaced by residues from a hypervariable region of a non-human species (donor antibody), such as mouse, rat, rabbit or non-human primate, having the desired specificity and affinity for a target antigen. This technique for creating humanized antibodies is often referred to as “CDR grafting.”

In some instances, additional modifications are made to further refine antibody performance. For example, framework region (FR) residues of the human immunoglobulin are replaced by corresponding non-human residues, or the humanized antibodies may comprise residues that are not found in either the recipient antibody or the donor antibody. In general, a variable domain in a humanized antibody will comprise all or substantially all of the hypervariable regions from a non-human immunoglobulin and all or substantially all of the FRs from a human immunoglobulin sequence. Humanized antibodies are described in more detail in Jones, et al., 1986, Nature, 321:522-525; Riechmann, et al., 1988, Nature, 332:323-329, and Presta, 1992, Curr. Op. Struct. Biol., 2:593-596, for example.

A number of approaches are known in the art for selecting the most appropriate human frameworks in which to graft the non-human CDRs. Early approaches used a limited subset of well-characterised human antibodies, irrespective of the sequence identity to the non-human antibody providing the CDRs (the “fixed frameworks” approach). More recent approaches have employed variable regions with high amino acid sequence identity to the variable regions of the non-human antibody providing the CDRs (“homology matching” or “best-fit” approach). An alternative approach is to select fragments of the framework sequences within each light or heavy chain variable region from several different human antibodies. CDR-grafting may in some cases result in a partial or complete loss of affinity of the grafted molecule for its target antigen. In such cases, affinity can be restored by back-mutating some of the residues of human origin to the corresponding non-human ones. Methods for preparing humanized antibodies by these approaches are well-known in the art (see, for example, Tsurushita & Vasquez, 2004, Humanization of Monoclonal Antibodies, Molecular Biology of B Cells, 533-545, Elsevier Science (USA); Jones et al., 1986, Nature, 321:522-525; Riechmann et al., 1988, Nature, 332:323-329; Presta et al., 1997, Cancer Res, 57(20):4593-4599).

Alternatively, or in addition to, these traditional approaches, more recent technologies may be employed to further reduce the immunogenicity of a CDR-grafted humanized antibody. For example, frameworks based on human germline sequences or consensus sequences may be employed as acceptor human frameworks rather than human frameworks with somatic mutation(s). Another technique that aims to reduce the potential immunogenicity of non-human CDRs is to graft only specificity-determining residues (SDRs). In this approach, only the minimum CDR residues required for antigen-binding activity (the “SDRs”) are grafted into a human germline framework. This method improves the “humanness” (i.e. the similarity to human germline sequence) of the humanized antibody and thus may help reduce the risk of immunogenicity of the variable region. These techniques have been described in various publications (see, for example, Almagro & Fransson, 2008, Front Biosci, 13:1619-1633; Tan, et al., 2002, J Immunol, 169:1119-1125; Hwang, et al., 2005, Methods, 36:35-42; Pelat, et al., 2008, J Mol Biol, 384:1400-1407; Tamura, et al., 2000, J Immunol, 164:1432-1441; Gonzales, et al., 2004, Mol Immunol, 1:863-872, and Kashmiri, et al., 2005, Methods, 36:25-34).

In certain embodiments, the anti-NaPi2b antibody construct of the present disclosure comprises humanized antibody sequences, for example, one or more humanized variable domains. In some embodiments, the anti-NaPi2b antibody construct can be a humanized antibody. Non-limiting examples of humanized antibodies based on the anti-NaPi2b antibody v23855 are described herein (see Examples and Sequence Tables and sequences for v29449, v29450, v29451, v29452, v29453, v29454, v29455, v29456, v29457, v29458, v29459, and v29460).

### Scaffolds

In certain embodiments, the anti-NaPi2b antibody constructs of the present disclosure comprise one or more antigen-binding domains operably linked to a scaffold. The antigen-binding domain(s) may be in one or a combination of the forms described above (for example, scFvs, Fabs and/or sdAbs). Examples of suitable scaffolds are described in more detail below and include, but are not limited to, immunoglobulin Fc regions, albumin, albumin analogues and derivatives, heterodimerizing peptides (such as leucine zippers, heterodimer-forming “zipper” peptides derived from Jun and Fos, IgG CH1 and CL domains or barnase-barstar toxins), cytokines, chemokines or growth factors. Other examples include antibodies based on the DOCK-AND-LOCK™ (DNL™) technology developed by IBC Pharmaceuticals, Inc. and Immunomedics, Inc. (see, for example, Chang, et al., 2007, Clin. Cancer Res., 13:5586s-5591s).

A scaffold may be a peptide, polypeptide, polymer, nanoparticle or other chemical entity. Where the scaffold is a polypeptide, each antigen-binding domain of the anti-NaPi2b antibody construct may be linked to either the N- or C-terminus of the polypeptide scaffold. Anti-NaPi2b antibody constructs comprising a polypeptide scaffold in which one or more of the antigen-binding polypeptide constructs are linked to a region other than the N- or C-terminus, for example, via the side chain of an amino acid with or without a linker, are also contemplated in certain embodiments.

In embodiments where the anti-NaPi2b antibody construct comprises a scaffold that is a peptide or polypeptide, the antigen-binding domain(s) may be linked to the scaffold by genetic fusion or chemical conjugation. Typically, when the scaffold is a peptide or polypeptide, the antigen-binding domain(s) are linked to the scaffold by genetic fusion. In some embodiments, where the scaffold is a polymer or nanoparticle, the antigen-binding domain(s) may be linked to the scaffold by chemical conjugation.

A number of protein domains are known in the art that comprise selective pairs of two different polypeptides and may be used to form a scaffold. An example is leucine zipper domains such as Fos and Jun that selectively pair together (Kostelny, et al., J Immunol, 148:1547-53 (1992); Wranik, et al., J. Biol. Chem., 287: 43331-43339 (2012)). Other selectively pairing molecular pairs include, for example, the barnase-barstar pair (Deyev, et al., Nat Biotechnol, 21:1486-1492 (2003)), DNA strand pairs (Chaudri, et al., FEBS Letters, 450(1-2):23-26 (1999)) and split fluorescent protein pairs (International Patent Application Publication No. WO 2011/135040).

Other examples of protein scaffolds include immunoglobulin Fc regions, albumin, albumin analogues and derivatives, toxins, cytokines, chemokines and growth factors. The use of protein scaffolds in combination with antigen-binding moieties has been described (see, for example, Müller et al., 2007, J. Biol. Chem., 282:12650-12660; McDonaugh et al., 2012, Mol. Cancer Ther., 11:582-593; Vallera et al., 2005, Clin. Cancer Res., 11:3879-3888; Song et al., 2006, Biotech. Appl. Biochem., 45:147-154, and U.S. Patent Application Publication No. 2009/0285816).

For example, fusing antigen-binding moieties such as scFvs, diabodies or single chain diabodies to albumin has been shown to improve the serum half-life of the antigen-binding moieties (Müller et al., ibid.). Antigen-binding moieties may be fused at the N- and/or C-termini of albumin, optionally via a linker.

Derivatives of albumin in the form of heteromultimers that comprise two transporter polypeptides obtained by segmentation of an albumin protein such that the transporter polypeptides self-assemble to form quasi-native albumin have been described (see International Patent Application Publication Nos. WO 2012/116453 and WO 2014/012082). As a result of the segmentation of albumin, the heteromultimer includes four termini and thus can be fused to up to four different antigen-binding moieties, optionally via linkers.

In certain embodiments, the anti-NaPi2b antibody construct may comprise a protein scaffold. In some embodiments, the anti-NaPi2b antibody construct may comprise a protein scaffold that is based on an immunoglobulin Fc region, an albumin or an albumin analogue or derivative. In some embodiments, the anti-NaPi2b antibody construct may comprise a protein scaffold that is based on an immunoglobulin Fc region, for example, an IgG Fc region.

### Fc Regions

The terms “Fe region,” “Fc” or “Fc domain” as used herein refer to a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, MD (1991).

In certain embodiments, the anti-NaPi2b antibody constructs of the present disclosure may comprise a scaffold that is based on an immunoglobulin Fc region. The Fc region may be dimeric and composed of two Fc polypeptides or alternatively, the Fc region may be composed of a single polypeptide.

An “Fe polypeptide” in the context of a dimeric Fc refers to one of the two polypeptides forming the dimeric Fc domain, i.e. a polypeptide comprising one or more C-terminal constant regions of an immunoglobulin heavy chain that is capable of stable self-association. When referring to a dimeric Fc region, the terms “first Fc polypeptide” and “second Fc polypeptide” may be used interchangeably provided that the Fc region comprises one first Fc polypeptide and one second Fc polypeptide.

An Fc region may comprise a CH3 domain or it may comprise both a CH3 and a CH2 domain. For example, in certain embodiments, an Fc polypeptide of a dimeric IgG Fc region may comprise an IgG CH2 domain sequence and an IgG CH3 domain sequence. In such embodiments, the CH3 domain comprises two CH3 sequences, one from each of the two Fc polypeptides of the dimeric Fc region, and the CH2 domain comprises two CH2 sequences, one from each of the two Fc polypeptides of the dimeric Fc region.

In some embodiments, the anti-NaPi2b antibody construct may comprise a scaffold that is based on an IgG Fc region. In some embodiments, the anti-NaPi2b antibody construct may comprise a scaffold that is based on a human IgG Fc region. In some embodiments, the anti-NaPi2b antibody construct may comprise a scaffold based on an IgG1 Fc region. In some embodiments, the anti-NaPi2b antibody construct may comprise a scaffold based on a human IgG1 Fc region.

In certain embodiments, the anti-NaPi2b antibody construct may comprise a scaffold based on an IgG Fc region, which is a heterodimeric Fc region, comprising a first Fc polypeptide and a second Fc polypeptide, each comprising a CH3 sequence, and optionally a CH2 sequence and in which the first and second Fc polypeptides are different. In some embodiments, the anti-NaPi2b antibody construct may comprise a scaffold based on an Fc region which comprises two CH3 sequences, at least one of which comprises one or more amino acid modifications. In some embodiments, the anti-NaPi2b antibody construct may comprise a scaffold based on an Fc region which comprises two CH3 sequences and two CH2 sequences, at least one of the CH2 sequences comprising one or more amino acid modifications.

In some embodiments, the anti-NaPi2b antibody construct may comprise a heterodimeric Fc region comprising a modified CH3 domain, where the modified CH3 domain is an asymmetrically modified CH3 domain comprising one or more asymmetric amino acid modifications. As used herein, an “asymmetric amino acid modification” refers to a modification, such as a substitution or an insertion, in which an amino acid at a specific position on a first CH3 or CH2 sequence is different to the amino acid on a second CH3 or CH2 sequence at the same position. These asymmetric amino acid modifications can be a result of modification of only one of the two amino acids at the same respective amino acid position on each sequence, or different modifications of both amino acids on each sequence at the same respective position on each of the first and second CH3 or CH2 sequences. Each of the first and second CH3 or CH2 sequences of a heterodimeric Fc may comprise one or more than one asymmetric amino acid modification.

In some embodiments, the anti-NaPi2b antibody construct may comprise a heterodimeric Fc comprising a modified CH3 domain, where the modified CH3 domain comprises one or more amino acid modifications that promote formation of the heterodimeric Fc over formation of a homodimeric Fc. In some embodiments, one or more of the amino acid modifications are asymmetric amino acid modifications.

Amino acid modifications that may be made to the CH3 domain of an Fc in order to promote formation of a heterodimeric Fc are known in the art and include, for example, those described in International Publication No. WO 96/027011 (“knobs into holes”), Gunasekaran et al., 2010, J Biol Chem, 285, 19637-46 (“electrostatic steering”), Davis et al., 2010, Prot Eng Des Sel, 23(4):195-202 (strand exchange engineered domain (SEED) technology) and Labrijn et al., 2013, Proc Natl Acad Sci USA, 110(13):5145-50 (Fab-arm exchange). Other examples include approaches combining positive and negative design strategies to produce stable asymmetrically modified Fc regions as described in International Publication Nos. WO 2012/058768 and WO 2013/063702. In certain embodiments, the anti-NaPi2b antibody construct may comprise a scaffold based on a modified Fc region as described in International Publication No. WO 2012/058768 or WO 2013/063702.

Table 5 provides the amino acid sequence of the human IgG1 Fc sequence (SEQ ID NO:16), corresponding to amino acids 231 to 447 of the full-length human IgG1 heavy chain. The CH3 sequence comprises amino acids 341-447 of the full-length human IgG1 heavy chain. Also shown in Table 5 are CH3 domain amino acid modifications that promote formation of a heterodimeric Fc as described in in International Patent Application Publication Nos. WO 2012/058768 and WO 2013/063702.

In certain embodiments, the anti-NaPi2b antibody construct may comprise a heterodimeric Fc scaffold having a modified CH3 domain comprising the modifications of any one of Variant 1, Variant 2, Variant 3, Variant 4 or Variant 5, as shown in Table 5.

TABLE 5
Human IgG1 Fc Sequence¹ and CH3 Domain Amino Acid Modifications
Promoting Heterodimer Formation

APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK (SEQ ID NO: 60)

| Variant No | Chain | Mutations |
|---|---|---|
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

¹Sequence from positions 231-447 (EU numbering)

In some embodiments, the anti-NaPi2b antibody construct may comprise a scaffold based on an Fc region comprising two CH3 sequences and two CH2 sequences, at least one of the CH2 sequences comprising one or more amino acid modifications. Modifications in the CH2 domain can affect the binding of Fc receptors (FcRs) to the Fc, such as receptors of the FcγRI, FcγRII and FcγRIII subclasses.

In some embodiments, the anti-NaPi2b antibody construct comprises a scaffold based on an IgG Fc having a modified CH2 domain, wherein the modification of the CH2 domain results in altered binding to one or more of the FcγRI, FcγRII and FcγRIII receptors.

A number of amino acid modifications to the CH2 domain that selectively alter the affinity of the Fc for different Fcγ receptors are known in the art. Amino acid modifications that result in increased binding and amino acid modifications that result in decreased binding can each be useful in certain indications. For example, increasing binding affinity of an Fc for FcγRIIIa (an activating receptor) may result in increased antibody dependent cell-mediated cytotoxicity (ADCC), which in turn results in increased lysis of the target cell. Decreased binding to FcγRIIb (an inhibitory receptor) likewise may be beneficial in some circumstances. In certain indications, a decrease in, or elimination of, ADCC and complement-mediated cytotoxicity (CDC) may be desirable. In such cases, modified CH2 domains comprising amino acid modifications that result in increased binding to FcγRIIb or amino acid modifications that decrease or eliminate binding of the Fc region to all of the Fcγ receptors (“knock-out” variants) may be useful.

Examples of amino acid modifications to the CH2 domain that alter binding of the Fc by Fcγ receptors include, but are not limited to, the following: S298A/E333A/K334A and S298A/E333A/K334A/K326A (increased affinity for FcγRIIIa) (Lu, et al., 2011, J Immunol Methods, 365(1-2):132-41); F243L/R292P/Y300L/V305I/P396L (increased affinity for FcγRIIIa) (Stavenhagen, et al. 2007, Cancer Res 67(18):8882-90); F243L/R292P/Y300L/L235V/P396L (increased affinity for FcγRIIIa) (Nordstrom J L, et al., 2011, Breast Cancer Res, 13(6):R123); F243L (increased affinity for FcγRIIIa) (Stewart, et al., 2011, Protein Eng Des Sel., 24(9):671-8); S298A/E333A/K334A (increased affinity for FcγRIIIa) (Shields, et al., 2001, J Biol Chem, 276(9):6591-604); S239D/I332E/A330L and S239D/I332E (increased affinity for FcγRIIIa) (Lazar, et al., 2006, Proc Natl Acad Sci USA, 103(11):4005-10), and S239D/S267E and S267E/L328F (increased affinity for FcγRIIb) (Chu, et al., 2008, Mol Immunol, 45(15):3926-33). Various amino acid modifications to the CH2 domain that alter binding of the Fc by FcγRIIb are described in International Publication No. WO 2021/232162. Additional modifications that affect Fc binding to Fcγ receptors are described in Therapeutic Antibody Engineering (Strohl & Strohl, Woodhead Publishing series in Biomedicine No 11, ISBN 1 907568 37 9, October 2012, page 283).

In certain embodiments, the anti-NaPi2b antibody construct comprises a scaffold based on an IgG Fc having a modified CH2 domain, in which the modified CH2 domain comprises one or more amino acid modifications that result in decreased or eliminated binding of the Fc region to all of the Fcγ receptors (i.e. a “knock-out” variant).

Various publications describe strategies that have been used to engineer antibodies to produce “knock-out” variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation, use of IgG2/IgG4 scaffolds, or the introduction of mutations in the hinge or CH2 domain of the Fc (see also, U.S. Patent Publication No. 2011/0212087, International Publication No. WO 2006/105338, U.S. Patent Publication No. 2012/0225058, U.S. Patent Publication No. 2012/0251531 and Strop et al., 2012, J. Mol. Biol., 420: 204-219).

Examples of mutations that may be introduced into the hinge or CH2 domain to produce a “knock-out” variant include the amino acid modifications L234A/L235A, and L234A/L235A/D265S.

In certain embodiments, the anti-NaPi2b antibody constructs described herein may comprise a scaffold based on an IgG Fc in which native glycosylation has been modified. As is known in the art, glycosylation of an Fc may be modified to increase or decrease effector function. For example, mutation of the conserved asparagine residue at position 297 to alanine, glutamine, lysine or histidine (i.e. N297A, Q, K or H) results in an aglycoslated Fc that lacks all effector function (Bolt et al., 1993, Eur. J. Immunol., 23:403-411; Tao & Morrison, 1989, J. Immunol., 143:2595-2601).

Conversely, removal of fucose from heavy chain N297-linked oligosaccharides has been shown to enhance ADCC, based on improved binding to FcγRIIIa (see, for example, Shields et al., 2002, J Biol Chem., 277:26733-26740, and Niwa et al., 2005, J. Immunol. Methods, 306:151-160). Such low fucose antibodies may be produced, for example in knockout Chinese hamster ovary (CHO) cells lacking fucosyltransferase (FUT8) (Yamane-Ohnuki et al., 2004, Biotechnol. Bioeng., 87:614-622); in the variant CHO cell line, Lee 13, that has a reduced ability to attach fucose to N297-linked carbohydrates (International Publication No. WO 03/035835), or in other cells that generate afucosylated antibodies (see, for example, Li et al., 2006, Nat Biotechnol, 24:210-215; Shields et al., 2002, ibid, and Shinkawa et al., 2003, J. Biol. Chem., 278:3466-3473). In addition, International Publication No. WO 2009/135181 describes the addition of fucose analogues to culture medium during antibody production to inhibit incorporation of fucose into the carbohydrate on the antibody.

Other methods of producing antibodies with little or no fucose on the Fc glycosylation site (N297) are well known in the art. For example, the GlymaX® technology (ProBioGen AG) (see von Horsten et al., 2010, Glycobiology, 20(12):1607-1618 and U.S. Pat. No. 8,409,572).

Other glycosylation variants include those with bisected oligosaccharides, for example, variants in which a biantennary oligosaccharide attached to the Fc region of the antibody is bisected by N-acetylglucosamine (GlcNAc). Such glycosylation variants may have reduced fucosylation and/or improved ADCC function (see, for example, International Publication No. WO 2003/011878, U.S. Pat. No. 6,602,684 and US Patent Application Publication No. US 2005/0123546). Useful glycosylation variants also include those having at least one galactose residue in the oligosaccharide attached to the Fc region, which may have improved CDC function (see, for example, International Publication Nos. WO 1997/030087, WO 1998/58964 and WO 1999/22764).

### Preparation of Anti-NaPi2b Antibody Constructs

The anti-NaPi2b antibody constructs described herein may be produced using standard recombinant methods known in the art (see, for example, U.S. Pat. No. 4,816,567 and “Antibodies: A Laboratory Manual,” 2^{nd} Edition, Ed. Greenfield, Cold Spring Harbor Laboratory Press, New York, 2014).

Typically, for recombinant production of an antibody construct, a polynucleotide or set of polynucleotides encoding the anti-NaPi2b antibody construct is generated and inserted into one or more vectors for further cloning and/or expression in a host cell. Polynucleotide(s) encoding the anti-NaPi2b antibody construct may be produced by standard methods known in the art (see, for example, Ausubel et al., Current Protocols in Molecular Biology, John Wiley & Sons, New York, 1994 & update, and “Antibodies: A Laboratory Manual,” 2^{nd} Edition, Ed. Greenfield, Cold Spring Harbor Laboratory Press, New York, 2014). As would be appreciated by one of skill in the art, the number of polynucleotides required for expression of the anti-NaPi2b antibody construct will be dependent on the format of the construct, including whether or not the antibody construct comprises a scaffold. For example, when an anti-NaPi2b antibody construct is in a monospecific mAb or FSA format, two polynucleotides each encoding one polypeptide chain will be required. When multiple polynucleotides are required, they may be incorporated into one vector or into more than one vector.

Generally, for expression, the polynucleotide or set of polynucleotides is incorporated into an expression vector or vectors together with one or more regulatory elements, such as transcriptional elements, which are required for efficient transcription of the polynucleotide. Examples of such regulatory elements include, but are not limited to, promoters, enhancers, terminators, and polyadenylation signals. One skilled in the art will appreciate that the choice of regulatory elements is dependent on the host cell selected for expression of the antibody construct and that such regulatory elements may be derived from a variety of sources, including bacterial, fungal, viral, mammalian or insect genes. The expression vector may optionally further contain heterologous nucleic acid sequences that facilitate expression or purification of the expressed protein. Examples include, but are not limited to, signal peptides and affinity tags such as metal-affinity tags, histidine tags, avidin/streptavidin encoding sequences, glutathione-S-transferase (GST) encoding sequences and biotin encoding sequences. The expression vector may be an extrachromosomal vector or an integrating vector.

Suitable host cells for cloning or expression of the anti-NaPi2b antibody constructs include various prokaryotic or eukaryotic cells as known in the art. Eukaryotic host cells include, for example, mammalian cells, plant cells, insect cells and yeast cells (such as Saccharomyces or Pichia cells). Prokaryotic host cells include, for example, E. coli, A. salmonicida or B. subtilis cells.

In certain embodiments, the anti-NaPi2b antibody construct may be produced in bacteria, in particular when glycosylation and Fc effector function are not needed, as described for example in U.S. Pat. Nos. 5,648,237; 5,789,199, and 5,840,523, and in Charlton, Methods in Molecular Biology, Vol. 248, pp. 245-254, B.K.C. Lo, ed., Humana Press, Totowa, N.J., 2003.

Eukaryotic microbes such as filamentous fungi or yeast may be suitable expression host cells in certain embodiments, in particular fungi and yeast strains whose glycosylation pathways have been “humanized” resulting in the production of an antibody construct with a partially or fully human glycosylation pattern (see, for example, Gerngross, 2004, Nat. Biotech. 22:1409-1414, and Li et al., 2006, Nat. Biotech. 24:210-215).

Suitable host cells for the expression of glycosylated anti-NaPi2b antibody constructs are usually eukaryotic cells. For example, U.S. Pat. Nos. 5,959,177, 6,040,498, 6,420,548, 7,125,978 and 6,417,429 describe PLANTIBODIES™ technology for producing antigen-binding constructs in transgenic plants. Mammalian cell lines adapted to grow in suspension may be particularly useful for expression of antibody constructs. Examples include, but are not limited to, monkey kidney CV1 line transformed by SV40 (COS-7), human embryonic kidney (HEK) line 293 or 293 cells (see, for example, Graham et al., 1977, J. Gen Virol., 36:59), baby hamster kidney cells (BHK), mouse sertoli TM4 cells (see, for example, Mather, 1980, Biol Reprod, 23:243-251), monkey kidney cells (CV1), African green monkey kidney cells (VERO-76), human cervical carcinoma (HeLa) cells, canine kidney cells (MDCK), buffalo rat liver cells (BRL 3A), human lung cells (W138), human liver cells (Hep G2), mouse mammary tumour (MMT 060562), TRI cells (see, for example, Mather et al., 1982, Annals N.Y. Acad Sci, 383:44-68), MRC 5 cells, FS4 cells, Chinese hamster ovary (CHO) cells (including DHFR⁻ CHO cells, see Urlaub et al., 1980, Proc Natl Acad Sci USA, 77:4216), and myeloma cell lines (such as Y0, NS0 and Sp2/0). Exemplary mammalian host cell lines suitable for production of antibody constructs are reviewed in Yazaki & Wu, Methods in Molecular Biology, Vol. 248, pp. 255-268 (B.K.C. Lo, ed., Humana Press, Totowa, N.J., 2003).

In certain embodiments, the host cell may be a transient or stable higher eukaryotic cell line, such as a mammalian cell line. In some embodiments, the host cell may be a mammalian HEK293T, CHO, HeLa, NS0 or COS cell line, or a cell line derived from any one of these cell lines. In some embodiments, the host cell may be a stable cell line that allows for mature glycosylation of the antibody construct.

The host cells comprising the expression vector(s) encoding the anti-NaPi2b antibody construct may be cultured using routine methods to produce the anti-NaPi2b antibody construct. Alternatively, in some embodiments, host cells comprising the expression vector(s) encoding the anti-NaPi2b antibody construct may be used therapeutically or prophylactically to deliver the anti-NaPi2b antibody construct to a subject, or polynucleotides or expression vectors may be administered to a cell from a subject ex vivo and the cell then returned to the body of the subject.

Typically, the anti-NaPi2b antibody constructs are purified after expression. Proteins may be isolated or purified in a variety of ways known to those skilled in the art (see, for example, Protein Purification: Principles and Practice, 3^{rd} Ed., Scopes, Springer-Verlag, NY, 1994). Standard purification methods include chromatographic techniques, including ion exchange, hydrophobic interaction, affinity, sizing or gel filtration, and reverse-phase, carried out at atmospheric pressure or at high pressure using systems such as FPLC and HPLC. Additional purification methods include electrophoretic, immunological, precipitation, dialysis and chromatofocusing techniques. Ultrafiltration and diafiltration techniques, in conjunction with protein concentration, are also useful. As is well known in the art, a variety of natural proteins bind Fc and antibodies, and these proteins may be used for purification of certain antibody constructs. For example, the bacterial proteins A and G bind to the Fc region. Likewise, the bacterial protein L binds to the Fab region of some antibodies. Purification may also be enabled by a particular fusion partner. For example, antibodies may be purified using glutathione resin if a GST fusion is employed, Ni⁺² affinity chromatography if a His-tag is employed or immobilized anti-flag antibody if a flag-tag is used. The degree of purification necessary will vary depending on the use of the anti-NaPi2b antibody constructs. In some instances, no purification may be necessary.

In certain embodiments, the anti-NaPi2b antibody constructs are substantially pure. The term “substantially pure” (or “substantially purified”) when used in reference to an anti-NaPi2b antibody construct described herein, means that the antibody construct is substantially or essentially free of components that normally accompany or interact with the protein as found in its naturally occurring environment, such as a native cell, or a host cell in the case of recombinantly produced construct. In certain embodiments, an anti-NaPi2b antibody construct that is substantially pure is a protein preparation having less than about 30%, less than about 25%, less than about 20%, less than about 15%, less than about 10%, or less than about 5% (by dry weight) of contaminating protein.

Certain embodiments of the present disclosure relate to a method of making an anti-NaPi2b antibody construct comprising culturing a host cell into which one or more polynucleotides encoding the anti-NaPi2b antibody construct, or one or more expression vectors encoding the anti-NaPi2b antibody construct, have been introduced, under conditions suitable for expression of the anti-NaPi2b antibody construct, and optionally recovering the anti-NaPi2b antibody construct from the host cell (or from host cell culture medium).

### Post-Translational Modifications

In certain embodiments, the anti-NaPi2b antibody constructs described herein may comprise one or more post-translational modifications. Such post-translational modifications may occur in vivo, or they be conducted in vitro after isolation of the anti-NaPi2b antibody construct from the host cell.

Post-translational modifications include various modifications as are known in the art (see, for example, Proteins—Structure and Molecular Properties, 2nd Ed., T. E. Creighton, W. H. Freeman and Company, New York, 1993; Post-Translational Covalent Modification of Proteins, B. C. Johnson, Ed., Academic Press, New York, pgs. 1-12, 1983; Seifter et al., 1990, Meth. Enzymol., 182:626-646, and Rattan et al., 1992, Ann. N.Y. Acad. Sci., 663:48-62). In those embodiments in which the anti-NaPi2b antibody constructs comprise one or more post-translational modifications, the constructs may comprise the same type of modification at one or several sites, or it may comprise different modifications at different sites.

Examples of post-translational modifications include glycosylation, acetylation, phosphorylation, amidation, derivatization by known protecting/blocking groups, formylation, oxidation, reduction, proteolytic cleavage or specific chemical cleavage by cyanogen bromide, trypsin, chymotrypsin, papain, V8 protease or NaBH₄.

Other examples of post-translational modifications include, for example, addition or removal of N-linked or O-linked carbohydrate chains, chemical modifications of N-linked or 0-linked carbohydrate chains, processing of N-terminal or C-terminal ends, attachment of chemical moieties to the amino acid backbone, and addition or deletion of an N-terminal methionine residue resulting from prokaryotic host cell expression. Post-translational modifications may also include modification with a detectable label, such as an enzymatic, fluorescent, luminescent, isotopic or affinity label to allow for detection and isolation of the protein. Examples of suitable enzyme labels include, but are not limited to, horseradish peroxidase, alkaline phosphatase, beta-galactosidase and acetylcholinesterase. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride and phycoerythrin. Examples of luminescent materials include luminol, and bioluminescent materials such as luciferase, luciferin and aequorin. Examples of suitable radioactive materials include iodine, carbon, sulfur, tritium, indium, technetium, thallium, gallium, palladium, molybdenum, xenon and fluorine.

Additional examples of post-translational modifications include acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, gamma-carboxylation, GPI anchor formation, hydroxylation, iodination, methylation, myristylation, pegylation, prenylation, racemization, selenoylation, sulfation, transfer-RNA mediated addition of amino acids to proteins such as arginylation, and ubiquitination.

### Polynucleotides, Nucleotides and Host Cells

Certain embodiments of the present disclosure relate to an isolated polynucleotide or a set of polynucleotides encoding an anti-NaPi2b antibody construct described herein. A polynucleotide in this context may encode all or part of an anti-NaPi2b antibody construct.

The terms “nucleic acid,” “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers.

A polynucleotide that “encodes” a given polypeptide is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

Certain embodiments of the present disclosure relate to vectors (such as expression vectors) comprising one or more polynucleotides encoding an anti-NaPi2b antibody construct described herein. The polynucleotide(s) may be comprised by a single vector or by more than one vector. In some embodiments, the polynucleotides are comprised by a multicistronic vector.

Certain embodiments of the present disclosure relate to host cells comprising polynucleotide(s) encoding an anti-NaPi2b antibody construct described herein or one or more vectors comprising the polynucleotide(s). In some embodiments, the host cell is eukaryotic, for example, a Chinese Hamster Ovary (CHO) cell, a human embryonic kidney (HEK) cell or a lymphoid cell (e.g. Y0, NS0, Sp20 cell).

### Antibody-Drug Conjugates

Certain embodiments of the present disclosure relate to antibody-drug conjugates (ADCs) comprising an anti-NaPi2b antibody construct conjugated to one or more drug moieties, such as cytotoxins or immune modulators.

Typically, in an ADC, the anti-NaPi2b antibody construct is conjugated to a drug moiety via a linker, which may be a cleavable or non-cleavable linker. The anti-NaPi2b antibody construct may be conjugated to a single drug molecule, or it may be conjugated to multiple drug molecules. The number of drug molecules conjugated to a single anti-NaPi2b antibody construct is defined by the drug-to-antibody ratio (DAR). In certain embodiments, in the ADCs of the present disclosure, the DAR is in the range of from about 1 to about 12, or from about 2 to about 12, or from about 2 to about 8.

In certain embodiments, the ADCs comprising an anti-NaPi2b antibody construct have the general Formula I:

A-(L-(D)ₘ)ₙ (I)

where A is an anti-NaPi2b antibody construct as described herein; L is a linker; D is a drug moiety; m is an integer between 1 and about 8, and n is between 1 and about 12.

In certain embodiments in Formula I, m is between 1 and 6. In some embodiments, m is 1 or 2. In some embodiments, n is between about 1 and about 8, for example, between about 2 and about 8.

Various compounds known to be useful as cytotoxic or immunomodulatory ADC payloads may be employed as the drug moiety in the ADCs comprising the anti-NaPi2b antibody constructs. Examples include, but are not limited to, maytansinoids and maytansinoid analogues, benzodiazepines and pyrrolobenzodiazepines, duocarmycins such as CC-1065 and analogues thereof, calicheamicins and calicheamicin analogues, auristatins and auristatin analogues, hemiasterlins and hemiasterlin analogues, tubulysins and tubulysin analogues, amatoxins and amatoxin analogues, camptothecins and camptothecin analogues, eribulin, TLR agonists (such as agonists of TLR7 and/or TLR8) and STING agonists.

In certain embodiments, the drug moiety comprised by the ADCs of the present disclosure is an auristatin or auristatin analogue, a hemiasterlin or a hemiasterlin analogue, a camptothecin or camptothecin analogue, or eribulin.

Typically, in the ADCs of the present disclosure, the drug moiety is linked to the anti-NaPi2b antibody construct by a linker. Linkers are bifunctional or multifunctional moieties capable of linking one or more drug molecules to the antibody construct. In some embodiments, the linker may be bifunctional (or monovalent) such that it links a single drug molecule to a single site on the antibody construct. In some embodiments, the linker may be multifunctional (or polyvalent) such that it links more than one drug molecule to a single site on the antibody construct. Multifunctional linkers may also be used to link one drug molecule to more than one site on the antibody construct in some embodiments.

Attachment of a linker to an anti-NaPi2b antibody construct can be accomplished in a variety of ways, such as through surface lysines, reductive-coupling to oxidized carbohydrates, or through cysteine residues liberated by reducing interchain disulfide linkages. Alternatively, attachment of a linker to an anti-NaPi2b antibody construct may be achieved by modification of the antibody construct to include additional cysteine residues (see, for example, U.S. Pat. Nos. 7,521,541; 8,455,622 and 9,000,130) or non-natural amino acids that provide reactive handles, such as selenomethionine, p-acetylphenylalanine, formylglycine or p-azidomethyl-L-phenylalanine to allow for site-specific conjugation (see, for example, Hofer et al., 2009, Biochemistry, 48:12047-12057; Axup et al., 2012, PNAS, 109:16101-16106; Wu et al., 2009, PNAS, 106:3000-3005; Zimmerman et al., 2014, Bioconj. Chem., 25:351-361). A further option is the use of GlycoConnect™ technology (Synaffix BV, Nijmegen, Netherlands), which involves enzymatic remodelling of the antibody glycans to allow for attachment of a linker by metal-free click chemistry (see, for example, European Patent No. EP 2 911 699).

Linkers typically include a functional group capable of reacting with the target group or groups on the antigen binding construct and one or more functional groups capable of reacting with a target group on the drug moiety. Suitable functional groups are known in the art and include those described, for example, in Bioconjugate Techniques (G. T. Hermanson, 2013, Academic Press). Non-limiting examples of functional groups for reacting with free cysteines or thiols include maleimide, haloacetamide, haloacetyl, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Also useful in this context are “self-stabilizing” maleimides such as those described in Lyon et al., 2014, Nat. Biotechnol., 32:1059-1062. Non-limiting examples of functional groups for reacting with surface lysines and amines include activated esters such as N-hydroxysuccinamide (NHS) esters or sulfo-NHS esters, imido esters such as Traut's reagent, isothiocyanates, aldehydes and acid anhydrides such as diethylenetriaminepentaacetic anhydride (DTPA). Other examples include succinimido-1,1,3,3-tetra-methyluronium tetrafluoroborate (TSTU) and benzotriazol-1-yl-oxytripyrrolidinophosphonium hexafluorophosphate (PyBOP). Non-limiting examples of functional groups capable of reacting with an electrophilic group on the antibody construct or drug moiety (such as an aldehyde or ketone carbonyl group) include hydrazide, oxime, amino, hydrazine, thiosemicarbazone, hydrazine carboxylate and arylhydrazide.

In certain embodiments, a linker that includes a functional group that allows for bridging of two interchain cysteines on the antibody binding construct may be used, such as a ThioBridge™ linker (Badescu et al., 2014, Bioconjug. Chem., 25:1124-1136), a dithiomaleimide (DTM) linker (Behrens et al., 2015, Mol. Pharm., 12:3986-3998), a dithioaryl(TCEP)pyridazinedione-based linker (Lee et al., 2016, Chem. Sci., 7:799-802) or a dibromopyridazinedione-based linker (Maruani et al., 2015, Nat. Commun., 6:6645).

A variety of linkers for linking drugs to antibodies are known in the art, including hydrazone-, disulfide- and peptide-based linkers. Linkers may be cleavable or non-cleavable. A cleavable linker is typically susceptible to cleavage under intracellular conditions, for example, through lysosomal processes. Examples include linkers that are protease-sensitive, acid-sensitive or reduction-sensitive. Non-cleavable linkers by contrast, rely on the degradation of the antibody in the cell, which typically results in the release of an amino acid-linker-drug moiety.

An example of a cleavable linker that may be useful in certain embodiments is a peptide-containing linker cleavable by an intracellular protease, such as lysosomal protease or an endosomal protease. Examples include dipeptide-containing linkers, such as those comprising the dipeptides Val-Cit, Phe-Lys, Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Met-(D)Lys, Asn-(D)Lys, Pro-(D)Lys or Met-(D)Lys; tripeptide-containing linkers such as those comprising the tripeptides Met-Cit-Val, Gly-Cit-Val, (D)Phe-Phe-Lys or (D)Ala-Phe-Lys, and tetrapeptide-containing linkers such as those comprising the tetrapeptides Gly-Phe-Leu-Gly, Gly-Gly-Phe-Gly or Ala-Leu-Ala-Leu.

Additional useful cleavable linkers include disulfide-containing linkers and linkers hydrolyzable at a specific pH or within a pH range, such as hydrazone linkers. Examples of disulfide-containing linkers include, but are not limited to, N-succinimydyl-4-(2-pyridyldithio) butanoate (SPDB) and N-succinimydyl-4-(2-pyridyldithio)-2-sulfo butanoate (sulfo-SPDB). Disulfide-containing linkers may optionally include additional groups to provide steric hindrance adjacent to the disulfide bond in order to improve the extracellular stability of the linker, for example, inclusion of a geminal dimethyl group. Linkers comprising combinations of these functionalities may also be useful, for example, linkers comprising both a hydrazone and a disulfide are known in the art.

A further example of a cleavable linker is a linker comprising a β-glucuronide, which is cleavable by β-glucuronidase, an enzyme present in lysosomes and tumor interstitium (see, for example, De Graaf et al., 2002, Curr. Pharm. Des., 8:1391-1403).

Cleavable linkers may optionally further comprise one or more additional functionalities such as self-immolative/self-elimination groups, stretchers or hydrophilic moieties.

Self-immolative/self-elimination groups that find use in linkers include, for example, p-aminobenzyloxycarbonyl (PABC) and p-aminobenzyl ether (PABE) groups, and methylated ethylene diamine (MED). Other examples of self-immolative groups include, but are not limited to, aromatic compounds that are electronically similar to the PABC or PABE group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., 1995, Chemistry Biology, 2:223-227) and 2-aminophenylpropionic acid amides (Amsberry, et al., 1990, J. Org. Chem., 55:5867-5877). Self-immolative/self-elimination groups, alone or in combination, are often included in peptide-based linkers but may also be included in other types of linkers. In some embodiments, the linker may include one or more self-immolative/self-elimination groups, for example, a PABC group, a PABE group, or a combination of a PABC or PABE group and an MED.

Stretchers that find use in linkers for ADCs include, for example, alkylene groups and stretchers based on aliphatic acids, diacids, amines or diamines, such as diglycolate, malonate, caproate and caproamide. Other stretchers include, for example, glycine-based stretchers and polyethylene glycol (PEG) or monomethoxy polyethylene glycol (mPEG) stretchers. PEG and mPEG stretchers also function as hydrophilic moieties and may be particularly useful with hydrophobic drugs, although their use in linkers with other drugs is also contemplated in some embodiments.

ADCs comprising an anti-NaPi2b antibody construct may be prepared by one of several routes known in the art, employing standard organic chemistry reactions, conditions, and reagents (see, for example, Bioconjugate Techniques (G. T. Hermanson, 2013, Academic Press). For example, conjugation may be achieved by (1) reaction of a functional group of an antibody construct with a bivalent linker reagent, to form antibody-linker intermediate Ab-L, via a covalent bond, followed by reaction with an activated drug moiety D; or (2) reaction of a functional group of a drug moiety with a linker reagent, to form drug-linker intermediate D-L, via a covalent bond, followed by reaction with a functional group of an antibody construct. Conjugation methods (1) and (2) may be employed with a variety of antibody constructs, drug moieties, and linkers to prepare the ADCs described here.

Various prepared linkers, linker components and drugs are commercially available or may be prepared using standard synthetic organic chemistry techniques (see, for example, March's Advanced Organic Chemistry (Smith & March, 2006, Sixth Ed., Wiley); Toki et al., 2002, J. Org. Chem., 67:1866-1872; Frisch et al., 1997, Bioconj. Chem., 7:180-186; Bioconjugate Techniques (G. T. Hermanson, 2013, Academic Press), and Antibody-Drug Conjugates: Methods in Molecular Biology (Ducry (Ed.), 2013, Springer)). In addition, a number of pre-formed drug-linkers suitable for reaction with a selected antibody construct are also available commercially, for example, drug-linkers comprising DM1, DM4, MMAE, MMAF or Duocarmycin SA are available from Creative BioLabs (Shirley, NY). Various antibody drug conjugation services are also available commercially from companies such as Lonza Inc. (Allendale, NJ), Abzena PLC (Cambridge, UK), ADC Biotechnology (St. Asaph, UK), Baxter BioPharma Solutions (Baxter Healthcare Corporation, Deerfield, IL) and Piramal Pharma Solutions (Grangemouth, UK).

The ADCs, once prepared, may be purified by standard techniques such as chromatography (for example, HPLC, size-exclusion, adsorption, ion exchange and/or affinity capture), dialysis and/or tangential flow filtration.

### Methods of Use

Certain aspects of the present disclosure relate to the therapeutic or diagnostic use of the anti-NaPi2b antibody constructs and ADCs. NaPi2b is overexpressed in a variety of cancers and certain embodiments of the present disclosure thus relate to the methods of using the anti-NaPi2b antibody constructs and ADCs in the treatment or diagnosis of an NaPi2b-positive cancer.

Examples of cancers which may be treated in certain embodiments are carcinomas, including adenocarcinomas and squamous cell carcinomas; melanomas and sarcomas. Carcinomas and sarcomas are also frequently referred to as “solid tumors.” Examples of commonly occurring solid tumors that may be treated in certain embodiments include, but are not limited to, brain cancer, breast cancer, cervical cancer, colon cancer, head and neck cancer, kidney cancer, lung cancer, ovarian cancer, pancreatic cancer, prostate cancer, stomach cancer, uterine cancer, non-small cell lung cancer (NSCLC) and colorectal cancer. Various forms of lymphoma also may result in the formation of a solid tumor and, therefore, may also be considered to be solid tumors in certain situations. Typically, the cancer to be treated is an NaPi2b-expressing cancer.

Certain embodiments relate to methods of inhibiting the growth of NaPi2b-positive tumor cells comprising contacting the cells with an anti-NaPi2b antibody construct or ADC described herein. The cells may be in vitro or in vivo. In certain embodiments, the anti-NaPi2b antibody constructs and ADCs may be used in methods of treating an NaPi2b-positive cancer or tumor in a subject.

Cancers that overexpress NaPi2b are typically solid tumors. Examples include, but are not limited to, ovarian cancer, endometrial cancer, and lung cancers (such as non-small cell lung cancer (NSCLC)).

Treatment of an NaPi2b-positive cancer may result in one or more of alleviation of symptoms, shrinking the size of the tumor, inhibiting growth of the tumor, diminishing one or more direct or indirect pathological consequences of the disease, preventing metastasis, decreasing the rate of disease progression, amelioration or palliation of the disease state, improving survival, increasing progression-free survival, remission and/or improving prognosis.

In certain embodiments, when used in the treatment of cancer, the anti-NaPi2b antibody constructs and ADCs may be administered systemically to the subject to be treated, for example, by bolus injection or continuous infusion into the subject's bloodstream. In certain embodiments, when used in the treatment of cancer, the anti-NaPi2b antibody constructs and ADCs may be administered to the subject locally at the site to be treated.

It is contemplated that the anti-NaPi2b antibody constructs and ADCs may be used alone or in combination with one or more known chemotherapeutic or immunotherapeutic agents typically used in the treatment of cancer. Combinations of the anti-NaPi2b antibody constructs or ADCs with standard chemotherapeutics or immunotherapeutics may act to improve the efficacy of the chemotherapeutic or immunotherapeutic and, therefore, may improve standard cancer therapies. This application can be important in the treatment of drug-resistant cancers which are not responsive to standard treatment. When used in conjunction with one or more known chemotherapeutic or immunotherapeutic agents, the anti-NaPi2b antibody construct or ADC may be administered prior to, or after, administration of the chemotherapeutic or immunotherapeutic agents, or they can be administered concomitantly.

The dosage of the anti-NaPi2b antibody construct or ADC to be administered is not subject to defined limits, but it will be a therapeutically effective amount. A “therapeutically effective amount” refers to that amount of an anti-NaPi2b antibody construct or ADC described herein which, when administered to a subject, is sufficient to effect a treatment of the particular indication. A therapeutically effective amount of anti-NaPi2b antibody construct or ADC in respect of cancer treatment may, for example, have one or more of the following effects: reduce the number of cancer cells, reduce the tumor size, inhibit cancer cell infiltration into peripheral organs, inhibit tumor metastasis, inhibit tumor growth; increase survival time and/or relieve to some extent one or more of the symptoms associated with the cancer. For cancer therapy, efficacy may alternatively be measured, for example, by assessing the time to disease progression (TTP) and/or determining the response rate (RR).

Certain embodiments relate to methods of detecting the presence of NaPi2b in a biological sample, such as a sample comprising cells or tissue, using an anti-NaPi2b antibody construct described herein. In some embodiments, the biological sample may have been taken from a patient, for example, a patient known or suspected to have a cancer. Some embodiments relate to methods of detecting the presence of NaPi2b in a biological sample that comprise contacting the sample with an anti-NaPi2b antibody construct described herein.

Certain embodiments relate to methods of diagnosing a disorder associated with increased expression of NaPi2b, such as a cancer, using an anti-NaPi2b antibody construct described herein. The method of diagnosis may be an in vivo method in which the anti-NaPi2b antibody construct is administered to the subject, or it may be an in vitro method in which a sample taken from the subject is contacted with the anti-NaPi2b antibody construct. For in vivo methods, administration may be systemic or local.

In methods of detecting the presence of NaPi2b or diagnosing a disorder associated with increased expression of NaPi2b, the anti-NaPi2b antibody construct may be labelled with a detectable label, such as a fluorescent, luminescent, chromophoric, chemiluminescent, radioactive or enzymatic label as is known in the art.

### Pharmaceutical Compositions

For therapeutic use, the anti-NaPi2b antibody constructs and ADCs may be provided in the form of pharmaceutical compositions comprising the anti-NaPi2b antibody construct or ADC and a pharmaceutically acceptable carrier or diluent. The compositions may be prepared by known procedures using well-known and readily available ingredients.

Pharmaceutical compositions may be formulated for administration to a subject by, for example, parenteral, oral (including, for example, buccal or sublingual), topical, rectal or vaginal routes, or by inhalation or spray. The term “parenteral” as used herein includes subcutaneous injection, and intradermal, intra-articular, intravenous, intramuscular, intravascular, intrasternal or intrathecal injection or infusion. The pharmaceutical composition will typically be formulated in a format suitable for administration to the subject, for example, as a syrup, elixir, tablet, troche, lozenge, hard or soft capsule, pill, suppository, oily or aqueous suspension, dispersible powder or granule, emulsion, injectable or solution. Pharmaceutical compositions may be provided as unit dosage formulations.

In certain embodiments, pharmaceutical compositions comprising the anti-NaPi2b antibody constructs or ADCs may be formulated for parenteral administration by infusion or in a unit dosage injectable form, for example as lyophilized formulations or aqueous solutions.

Pharmaceutically acceptable carriers are generally nontoxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 residues) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, pharmaceutical compositions comprising the anti-NaPi2b antibody constructs or ADCs may be in the form of a sterile injectable aqueous or oleaginous solution or suspension. Such suspensions may be formulated using suitable dispersing or wetting agents and/or suspending agents that are known in the art. The sterile injectable solution or suspension may comprise the anti-NaPi2b antibody construct or ADC in a non-toxic parentally acceptable diluent or solvent. Acceptable diluents and solvents that may be employed include, for example, 1,3-butanediol, water, Ringer's solution or isotonic sodium chloride solution. In addition, sterile, fixed oils may be employed as a solvent or suspending medium. For this purpose, various bland fixed oils may be employed, including synthetic mono- or diglycerides. In addition, fatty acids such as oleic acid find use in the preparation of injectables. Adjuvants such as local anesthetics, preservatives and/or buffering agents may also be included in the injectable solution or suspension.

In certain embodiments, pharmaceutical compositions comprising the anti-NaPi2b antibody constructs or ADCs may be formulated for intravenous administration to a subject, for example a human. Typically, compositions for intravenous administration are solutions in sterile isotonic aqueous buffer. Where necessary, the composition may also include a solubilizing agent and/or a local anaesthetic such as lignocaine to ease pain at the site of the injection. Generally, the ingredients are supplied either separately or mixed together in unit dosage form, for example, as a dry lyophilized powder or water-free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Pharmaceutical Kits

Certain embodiments relate to pharmaceutical kits comprising an anti-NaPi2b antibody construct or ADC as described herein.

The kit typically will comprise a container holding the anti-NaPi2b antibody construct or ADC and a label and/or package insert on or associated with the container. The label or package insert contains instructions customarily included in commercial packages of therapeutic products, providing information about the indications, usage, dosage, administration, contraindications and/or warnings concerning the use of such therapeutic products. The label or package insert may further include a notice in the form prescribed by a governmental agency regulating the manufacture, use or sale of pharmaceuticals or biological products, which notice reflects approval by the agency of manufacture, for use or sale for human or animal administration. In some embodiments, the container may have a sterile access port. For example, the container may be an intravenous solution bag or a vial having a stopper that may be pierced by a hypodermic injection needle.

In addition to the container holding the anti-NaPi2b antibody construct or ADC, the kit may optionally comprise one or more additional containers comprising other components of the kit. For example, a pharmaceutically acceptable buffer (such as bacteriostatic water for injection (BWFI), phosphate-buffered saline, Ringer's solution or dextrose solution), other buffers or diluents.

Suitable containers include, for example, bottles, vials, syringes, intravenous solution bags, and the like. The containers may be formed from a variety of materials such as glass or plastic. If appropriate, one or more components of the kit may be lyophilized or provided in a dry form, such as a powder or granules, and the kit can additionally contain a suitable solvent for reconstitution of the lyophilized or dried component(s).

The kit may further include other materials desirable from a commercial or user standpoint, such as filters, needles, and syringes.

The following Examples are provided for illustrative purposes and are not intended to limit the scope of the invention in any way.

### EXAMPLES

### Example 1: Preparation of Anti-NaPi2b Antibodies

Antibody constructs that specifically bind human NaPi2b were generated by immunizing mice with human cells over-expressing NaPi2b as summarized below.

HEK293-6E cells (National Research Council of Canada) were transiently transfected with a pTT5-based expression plasmid (National Research Council of Canada) encoding human NaPi2b (pTT5-huNaPi2b, expressing the sequence of NaPi2b as set forth in SEQ ID NO:1), according to manufacturer's instructions for Lipofectamine 2000 (Thermo Fisher Scientific). Ten B6x 129 mice were subcutaneously immunized with transfected HEK293-6E cells over 63 days, after which blood was drawn and spleens harvested.

Anti-human NaPi2b antibody titers were determined by flow cytometry using CHO-S cells expressing human NaPi2b. All ten mice mounted a significant response against human NaPi2b.

Splenocytes from all mice were subsequently pooled and used for hybridoma generation. P3X63Ag8.653 cells (ATCC cat #CRL-1580) were mixed with IgG+ B cells isolated from spleens and fused using an ECM 2001 electrofusion instrument (BTX, Harvard Bioscience) with optimized settings. Following overnight recovery, hybridomas were diluted and plated in selection media containing the following final concentrations of substituents: 100 μM hypoxanthine, 0.4 μM aminopterin and 16 μM thymidine. Following 14 days of selection, hybridomas were diluted to an average of one cell per well and plated into 96-well plates. Cell supernatants containing secreted antibodies were assessed for binding on CHO-S cells transfected with the same plasmid used to transfect HEK293-6E cells (pTT5-huNaPi2b). Hybridoma cells from wells containing supernatants having antibodies that bound NaPi2b were harvested for sequencing.

The murine VH and VL sequences for one of the anti-NaPi2b antibodies identified were used to prepare a mouse-human chimeric IgG1/kappa antibody construct, v23855, as follows. Coding sequences for antibody variable regions were cloned in frame into a human IgG1 expression vector (with human IgG1 constant region starting with alanine 118 according to Kabat numbering) or a human C kappa expression vector (with human C kappa constant region starting at arginine 108 according to Kabat numbering), both expression vectors based on pTT5. The activities of the resultant recombinant chimeric antibody construct were confirmed in specificity binding assays and were found comparable to the parental antibodies (data not shown).

### Example 2: Humanization of Anti-NaPi2b Antibody Constructs

The chimeric anti-human NaPi2b antibody construct, variant v23855, generated as described in Example 1, was humanized. The CDR sequences of v23855 are provided in Table 2.1, and the mouse VH and VL sequences are provided in Table 2.2. Humanization was conducted as described below.

TABLE 2.1
CDR Sequences of the Anti-NaPi2b Antibody Construct v23855

| Numbering | | | | | | |
|---|---|---|---|---|---|---|
| System | Sequence | | | | | |
| | | SEQ | | SEQ | | SEQ |
| | Heavy Chain | ID | Heavy Chain | ID | Heavy Chain | ID |
| | CDR1 | NO | CDR2 | NO | CDR3 | NO |
| IMGT | GFTFTSYN | 4 | ISPGNGDL | 5 | ARGRTGMGAVDY | 6 |
| Kabat | SYNVH | 7 | AISPGNGDLS | 8 | GRTGMGAVDY | 9 |
| | | | YNQRFKD | | | |
| Chothia | GFTFTSY | 10 | SPGNGD | 11 | GRTGMGAVDY | 9 |
| AbM | GFTFTSYNVH | 12 | AISPGNGDLS | 13 | GRTGMGAVDY | 9 |
| Contact | TSYNVH | 14 | WIAAISPGNG | 15 | ARGRTGMGAVD | 16 |
| | | | DLS | | | |
| | | SEQ | | SEQ | | SEQ |
| | Light Chain | ID | Light Chain | ID | Light Chain | ID |
| | CDR1 | NO | CDR2 | NO | CDR3 | NO |
| IMGT | QDVYSY | 17 | YTS | — | QQYNIFPWT | 18 |
| Kabat | RASQDVYSYLN | 19 | YTSRLQS | 20 | QQYNIFPWT | 18 |
| Chothia | RASQDVYSYLN | 19 | YTSRLQS | 20 | QQYNIFPWT | 18 |
| AbM | RASQDVYSYLN | 19 | YTSRLQS | 20 | QQYNIFPWT | 18 |
| Contact | YSYLNWY | 21 | LLIYYTSRLQ | 22 | QQYNIFPW | 23 |

TABLE 2.2
VH and VL Sequences of the Anti-NaPi2b Antibody Construct v23855

| | | SEQ ID |
|---|---|---|
| | Sequence | NO |
| VH | QAYLQQSGAELVRPGASVKMSCKASGFTFTSYNVHWVKQTPRQGLEWI | 31 |
| | AAISPGNGDLSYNQRFKDKATLTVDRSSSTAYMQLSGLTSEDSAVYFCAR | |
| | GRTGMGAVDYWGQGTSVTVSS | |
| VL | DVQMTQTASSLSASLGDRVTISCRASQDVYSYLNWYQQKPHGTVKLLIY | 32 |
| | YTSRLQSGVPSRFTGSGSGTDYSLTISNLEPEDIATYYCQQYNIFPWTFGG | |
| | GTKLEIK | |

### 2.1 Humanization

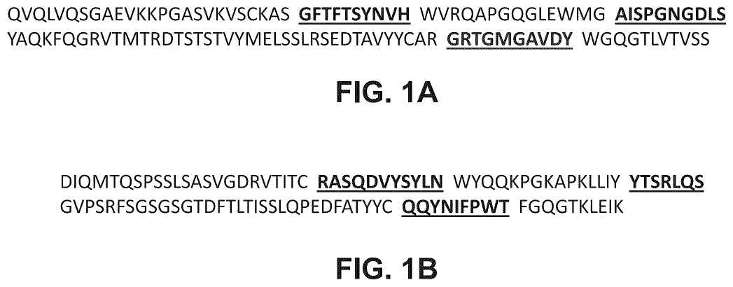

Sequence alignment of the mouse VH and VL sequences of v23855 to respective human germline sequences identified IGHV1-46*03 and IGKVID-39*01 as the closest, as well as most frequent, human germline sequences (and respectively IGHJ4*03 and IGKJ2*04 joining region germline sequences were selected). CDR sequences according to the AbM definition (see Table 2.1) were ported onto the framework of these selected human germline sequences as shown in FIG. 1A and FIG. 1B. Back mutations to mouse residues in the resultant sequences at positions judged likely to be important for the retention of binding affinity to antigen, NaPi2b, were included creating several humanized sequences in which generated sequences were built on the previous sequence, and where the first humanized sequence contained no back mutations. None of the variants modified the CDRs of the parent antibody as defined by the AbM method.

This process resulted in four variable heavy chain humanized sequences and three variable light chain humanized sequences. Full heavy chain sequences containing humanized heavy chain variable domain (VH) and hIgG1 heavy chain constant domains (CH1, hinge, CH2, CH3), and full light chain sequence containing humanized light chain variable domain (VL) and human kappa light chain constant domain (kappa CL) were assembled. Monoclonal antibody (mAb) variants were then assembled such that each of the humanized heavy chains was paired with each of the humanized light chains to provide 12 humanized variants to be evaluated experimentally.

### 2.2 Production of Humanized Antibody Constructs

Each of the 12 humanized antibody constructs, as well as the parental v23855 construct, were produced in full-size antibody (FSA) format containing two identical full-length heavy chains and two identical kappa light chains.

The full-length heavy chain contained the human CH1-hinge-CH2-CH3 domain sequence of IGHG1*01 [SEQ ID NO:33]; (see Table 2.3). The light chain contained the human kappa CL sequence of IGKC*01 [SEQ ID NO:34]; (see Table 2.3).

TABLE 2.3
Constant Heavy and Light Chain Sequences

| | | SEQ ID |
|---|---|---|
| | Sequence | NO |
| CH1-hinge- | ASTKGPSVFPLAPSSKSTSGGTAALGCLVKDYFPEPVTVSWNSGALTS | 33 |
| CH2-CH3 | GVHTFPAVLQSSGLYSLSSVVTVPSSSLGTQTYICNVNHKPSNTKVDK | |
| (IGHG1*01) | KVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSRD | |
| | ELTKNQVSLTCLVKGFYPSDIAVEWESNGQPENNYKTTPPVLDSDGSF | |
| | FLYSKLTVDKSRWQQGNVFSCSVMHEALHNHYTQKSLSLSPG | |
| CL (kappa) | RTVAAPSVFIFPPSDEQLKSGTASVVCLLNNFYPREAKVQWKVDNALQ | 34 |
| (IGKC*01) | SGNSQESVTEQDSKDSTYSLSSTLTLSKADYEKHKVYACEVTHQGLSS | |
| | PVTKSFNRGEC | |

Each of the humanized VH domain sequences as well as the mouse VH domain sequence was appended to the human CH1-hinge-CH2-CH3 domain sequence of IGHG1*01 to provide four humanized full heavy chain sequences and one parental mouse-human chimeric full heavy chain sequence. Each of the humanized VL domain sequences or mouse VL domain sequence was appended to the human kappa CL sequence of IGKC*01 to provide three humanized light chain sequences and one parental mouse-human chimeric light chain sequence. All sequences were reverse translated to DNA, codon optimized for mammalian expression and gene synthesized.

Heavy chain vector inserts comprising a signal peptide (artificially designed sequence: MRPTWAWWLFLVLLLALWAPARG [SEQ ID NO:35](Barash et al., 2002, Biochem and Biophys Res. Comm., 294:835-842)) and the heavy chain clone terminating at residue G446 (EU numbering) of the CH3 domain were ligated into a pTT5 vector to produce heavy chain expression vectors. Light chain vector inserts comprising the same signal peptide were ligated into a pTT5 vector to produce light chain expression vectors. The resulting heavy and light chain expression vectors were sequenced to confirm correct reading frame and sequence of the coding DNA. Sequences of the humanized VH and VL sequences are provided in Table 2.4 below.

TABLE 2.4
Amino Acid Sequences of Humanized VH and VL Sequences

| | | SEQ ID |
|---|---|---|
| Name | Amino Acid Sequence | NO |
| H1 | QVQLVQSGAEVKKPGASVKVSCKASGFTFTSYNVHWVRQAPGQ | 24 |
| | GLEWMGAISPGNGDLSYAQKFQGRVTMTRDTSTSTVYMELSSL | |
| | RSEDTAVYYCARGRTGMGAVDYWGQGTLVTVSS | |
| H2 | QVQLVQSGAEVKKPGASVKVSCKASGFTFTSYNVHWVRQAPGQ | 25 |
| | GLEWIAAISPGNGDLSYAQKFQGRVTMTVDTSTSTVYMELSSLR | |
| | SEDTAVYYCARGRTGMGAVDYWGQGTLVTVSS | |
| H3 | QVQLVQSGAEVKKPGASVKMSCKASGFTFTSYNVHWVRQAPG | 26 |
| | QGLEWIAAISPGNGDLSYAQKFQGRATLTVDTSTSTAYMELSSLR | |
| | SEDTAVYYCARGRTGMGAVDYWGQGTLVTVSS | |
| H4 | QVQLVQSGAEVKKPGASVKMSCKASGFTFTSYNVHWVRQAPG | 27 |
| | QGLEWIAAISPGNGDLSYAQKFQGRATLTVDRSTSTAYMELSSL | |
| | RSEDTAVYYCARGRTGMGAVDYWGQGTLVTVSS | |
| L1 | DIQMTQSPSSLSASVGDRVTITCRASQDVYSYLNWYQQKPGKAP | 28 |
| | KLLIYYTSRLQSGVPSRFSGSGSGTDFTLTISSLQPEDFATYYCQQ | |
| | YNIFPWTFGQGTKLEIK | |
| L2 | DVQMTQSPSSLSASVGDRVTITCRASQDVYSYLNWYQQKPGKA | 29 |
| | VKLLIYYTSRLQSGVPSRFSGSGSGTDYTLTISSLQPEDFATYYCQ | |
| | QYNIFPWTFGQGTKLEIK | |
| L3 | DVQMTQSPSSLSASVGDRVTITCRASQDVYSYLNWYQQKPGKA | 30 |
| | VKLLIYYTSRLQSGVPSRFSGSGSGTDYTLTISSLQPEDIATYYCQ | |
| | QYNIFPWTFGQGTKLEIK | |

The heavy and light chains of each of the humanized antibody variants and parental mouse-human chimeric antibody variant were expressed in 300 mL cultures of CHO-3E7 cells. Briefly, CHO-3E7 cells, at a density of 1.7-2×10⁶ cells/mL, viability >95%, were cultured at 37° C. in FreeStyle™ F17 medium (Thermo Fisher Scientific, Waltham, MA) supplemented with 4 mM glutamine (Hyclone SH30034.01) and 0.1% Pluronic® F-68 (Gibco/Thermo Fisher Scientific, Waltham, MA). A total volume of 300 mL CHO-3E7 cells+1× antibiotic/antimycotics (GE Life Sciences, Marlborough, MA) was transfected with a total of 300 μg DNA (150 μg of antibody DNA and 150 μg of GFP/AKT/stuffer DNA) using PEI-MAX® (Polyscience, Inc., Philadelphia, PA) at a DNA:PEI ratio of 1:4 (w/w). Twenty-four hours after the addition of the DNA-PEI mixture, 0.5 mM valproic acid (final concentration)+1% w/v Tryptone (final concentration) were added to the cells, which were then transferred to 32° C. and incubated for 6 more days prior to harvesting.

Protein-A purification was performed using 1 mL HiTrap™ MabSelect™ SuRe™ columns (Cytiva, Marlborough, MA). Clarified supernatant samples were loaded on equilibrated columns in Dulbecco's PBS (DPBS). The columns were washed with DPBS. Proteins were eluted with 100 mM sodium citrate buffer pH 3.0. The eluted fractions were pH adjusted by adding 10% (v/v) 1M HEPES (pH ˜10.6-10.7) to yield a final pH of 6-7. Samples were buffer exchanged into DPBS using 5 mL Zeba™ Spin columns (Thermo Scientific). Protein was quantitated based on absorbance at 280 nm (A280 nm).

Following purification, purity of samples was assessed by SDS-PAGE under non-reducing and reducing conditions. Protein sample was mixed with NuPAGE® LDS Sample Buffer and NuPAGE® Sample Reducing Agent (for reducing condition only) according to manufacturer's protocol, after which sample was heated at 70° C. for 15 min. Treated protein samples containing 1.5 g of protein and Molecular Weight (MW) Precision Plus Protein™ Dual Color (Bio-Rad) standards for MW estimation were loaded on the NuPAGE 4-12% Bis-Tris gel (15 wells). XCell SureLock® Mini-Cell system from Life Technologies (Thermo Fisher Scientific) as well as NuPAGE® MOPS SDS Running Buffer, were used to perform gel electrophoresis at 200 V for 50 minutes. Gels were stained with a Biosafe Coomassie solution and ChemiDoc™ MP Imaging system (Bio-Rad) was used to capture images of gels.

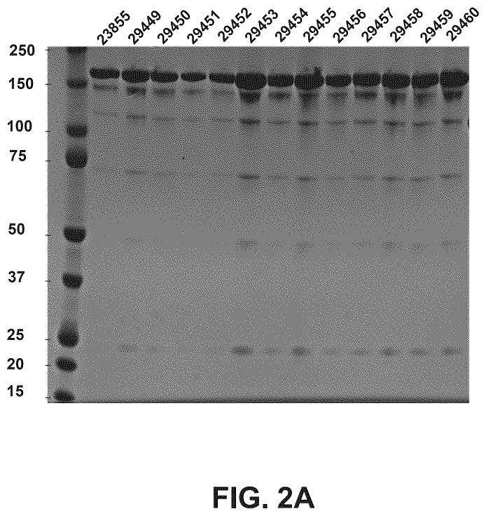

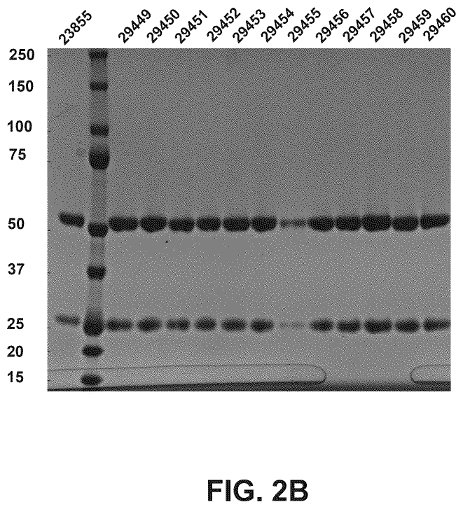

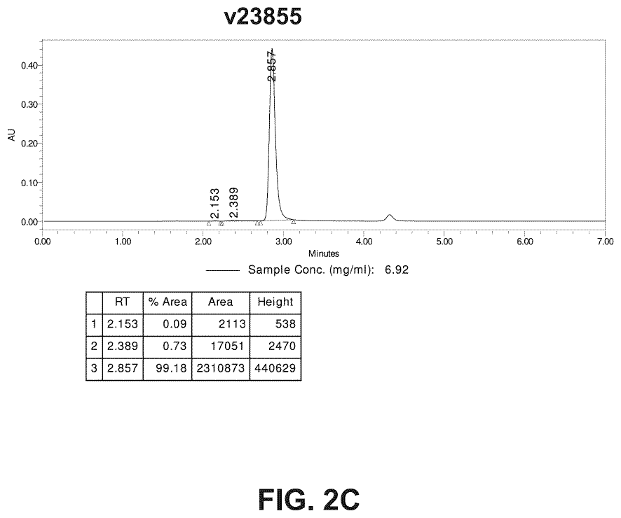

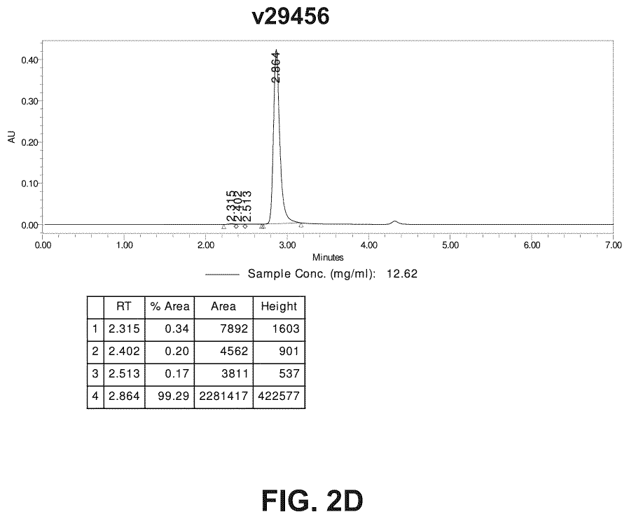

The yields for each of the twelve humanized antibody variants were similar, ranging from approximately 23-30 mg (or ˜77-100 mg/L of culture) and approximately 2-fold higher yield than the parental mouse-human chimeric antibody, v23855 (14 mg yield). The SDS-PAGE results for these antibody samples are shown in FIG. 2A (non-reducing) and FIG. 2B (reducing). As can be seen from these Figures, non-reducing (NR) and reducing (R) SDS-PAGE reflected a single species corresponding to full-size antibody and intact heavy and light chains.

### 2.3 Quality Assessment of Humanized Antibodies

Species homogeneity of the humanized antibody variants and parental mouse-human chimeric antibody variant samples was assessed by UPLC-SEC after protein-A purification.

UPLC-SEC was performed using a Waters Acquity BEH200 SEC column (2.5 mL, 4.6×150 mm, stainless steel, 1.7 m particles) (Waters LTD, Mississauga, ON) set to 30° C. and mounted on a Waters Acquity UPLC™ H-Class Bio system with a photodiode array (PDA) detector. The mobile phase was Dulbecco's phosphate buffered saline (DPBS) with 0.02% Tween 20 pH 7.4 and the flow rate was 0.4 mL/min. Total run time for each injection was 7 min with a total mobile phase volume of 2.8 mL. Elution was monitored by UV absorbance in the range 210-500 nm, and chromatograms were extracted at 280 nm. Peak integration was performed using Waters Empower® 3 software employing the Apex Track™ and detect shoulders features.

FIG. 2C and FIG. 2D show the UPLC-SEC profiles for the parental mouse-human chimeric antibody v23855 and representative humanized antibody v29456 samples. The UPLC-SEC profile for the representative humanized antibody sample reflected high species homogeneity, comparable to the parental mouse-human chimeric antibody sample. The samples from the remaining humanized antibody variants had similar profiles to that shown for the representative humanized antibody sample.

### 2.4 Purity Assessment of Humanized Antibodies

The apparent purity of the humanized antibody variants was assessed using mass spectrometry and non-denaturing deglycosylation.

10 μg of each sample were incubated with 1 μg of deglycosylation mix (NEB, P6044) for 1 hour at room temperature and transferred to the incubator at 37° C. for 16 hours.

After deglycosylation, 5 μL of the eluted sample were transferred into glass inserts in LC-MS vials. For LC-MS analysis, 1 μL of sample were injected into using an Agilent PLRP-S column (1000 Å, 2.1×50 mm, 8 μm) using an Agilent 1290 Infinity II LC system coupled to Agilent 6545 QTOF with Dual Jet Stream electrospray ionization source with a column temperature of 70° C. and a flow rate of 0.3 mL/mi. Mobile phases consisted of A: LC-MS grade water with 0.1% v/v formic acid, 0.025 v/v trifluoroacetic acid and 10% v/v isopropyl alcohol in, and B: acetonitrile with 0.1% v/v formic acid and 10% v/v isopropyl alcohol. The column was pre-equilibrated in 20% mobile phase B before injection. Then, a 20 minute 20 to 40% mobile phase B gradient was applied, followed by a 2 minute 27 to 90% mobile phase B gradient and a column wash of 2 minutes at 99% mobile phase B.

### Example 3: Characterization of Humanized Anti-Napi2b Antibody Constructs—Assessment of Thermal Stability

The thermal stability of the humanized antibody variants was assessed by differential scanning calorimetry (DSC) as described below.

400 μL of purified samples primarily at concentrations of 0.4 mg/mL in PBS were used for DSC analysis with a VP-Capillary DSC (Malvern Panalytical Inc., Westborough, MA). At the start of each DSC run, 5 buffer blank injections were performed to stabilize the baseline, and a buffer injection was placed before each sample injection for referencing. Each sample was scanned from 20° C. to 100° C. at a 60° C./hr rate, with low feedback, 8 sec filter, 3 min pre-scan thermostat, and 70 psi nitrogen pressure. The resulting thermograms were referenced and analyzed using Origin 7 software (OriginLab Corporation, Northampton, MA) to determine melting temperature (Tm) as an indicator of thermal stability.

The Fab Tm values determined for the humanized variants are shown in Table 3.1. All humanized variants exhibited increased thermal stability compared to the parental antibody, v23855 (Fab Tm of ˜72.4° C.), with Fab Tm values ranging from −78-83° C.

TABLE 3.1
Thermal Stability of Humanized Variants

| | Heavy and Light | Fab Tm |
|---|---|---|
| Variant | Chain Composition | (° C.) |
| v23855 | chimera | 72.4 |
| (parental) | | |
| v29449 | H4L3 | 80.0 |
| v29450 | H3L3 | 81.0 |
| v29451 | H2L3 | 83.0 |
| v29452 | H1L3 | 79.5 |
| v29453 | H4L2 | 78.9 |
| v29454 | H3L2 | 79.7 |
| v29455 | H2L2 | 81.5 |
| v29456 | H1L2 | 77.9 |
| v29457 | H4L1 | 80.5 |
| v29458 | H3L1 | 81.6 |
| v29459 | H2L1 | 83.1 |
| v29460 | H1L1 | 80.3 |

### Example 4: Functional Characterization of Anti-Napi2b Antibody Constructs—Competition Binding (Epitope Binning)

To characterize the binding of parental chimeric anti-NaPi2b antibody v23855 to NaPi2b, competition binding or epitope binning assays were carried out against anti-NaPi2b reference antibodies MX-35 (v18992) and lifastuzumab (v18993). Binding was assessed by flow cytometry using HEK293-6e cells as described below.

Each of the anti-NaPi2b detection antibodies: v23855, v18992, v18993, and Palivizumab (anti-RSV, v16955) was conjugated with AF647 fluorophores using Zenon Human IgG labeling kit (ThermoFisher Scientific Corporation, Waltham, MA; Cat. No. Z25408 Lot. No. 1937175). HEK293-6e cells were transfected for ˜24 hours to transiently express human NaPi2b (1 μg of pTT5-NaPi2b per 1 million cells) or transfected with GFP (ATUM, Menlo Park, CA; pD2610-v23, also 1 μg of DNA per 1 million cells). Following transfection, human NaPi2b-expressing HEK296-6e cells and transfected GFP-expressing HEK296-6e cells were mixed at 4:1 ratio. Each well of a V-bottom 96-well plate was seeded with 100,000 cells of the mixture and incubated with 100 μg/mL of unlabeled competitor anti-NaPi2b antibody for an hour on ice. Post incubation, cells were washed and stained with 1 μg/mL of AF647-conjugated anti-NaPi2b detection antibodies for an hour on ice. Following staining and washing, fluorescence was detected by flow cytometry on a BD LSRFortessa™ Cell Analyzer (BD Biosciences, Franklin Lake, NJ) with 10,000 minimum events collected per well. The AF647/APC-A GeoMean (fluorescence signal geometric mean, proportional to anti-Human AF647 binding) was calculated for the FITC/GFP negative live cell population and using FlowJo™ Version 10.8.1 (BD Biosciences, Franklin Lake, NJ). Percentage inhibition was calculated using the following formula:

$$\% inhibition of detection mAb = 100 - ( \frac{Competitor mAb GeoMean}{v 16955 GeoMean} * 100 )$$

The competition binding results of parental chimeric anti-NaPi2b antibody v23855 against v18992 (MX35) and v18993 (lifastuzumab) are shown in Table 4.1.

TABLE 4.1
Competition binding. Percentage inhibition (vs.
negative control antibody). GFP-population

| | | Labeled detection antibody (1 μg/mL) | | | |
|---|---|---|---|---|---|
| | | | | | v16955 |
| % Inhibition vs. | | | v18992 | v18993 | (negative |
| irrelevant mAb | | v23855 | (MX35) | (lifastuzumab) | control |
| Unlabeled | v23855 | 96 | 97 | 94 | −148 |
| competitor | v18992 | 95 | 97 | 95 | −135 |
| antibody | (MX35) | | | | |
| (100 μg/mL) | v18993 | 94 | 94 | 96 | −89 |
| | (lifastuzumab) | | | | |
| | v16955 | 0 | 0 | 0 | 0 |
| | (negative | | | | |
| | control) | | | | |
| | No mAb | −20 | −1 | −14 | −17 |

Each of the tested anti-NaPi2b antibodies competed against itself (>95% inhibition, see data in bold text) as expected. The chimeric anti-NaPi2b antibody v23855 competed for binding to v18992 and v18993, demonstrated by comparable % inhibition to itself (>94%). No competition binding against negative control palivizumab (v16955) was observed, as expected.

### Example 5: Functional Characterization of Humanized Anti-NaPi2b Antibody Constructs—Cynomolgus and Mouse Napi2b Binding

The binding cross-reactivity of humanized antibody variant v29456 to human, cynomolgus and mouse NaPi2b was assessed by flow cytometry using HEK293-6e transfected cells. Reference anti-NaPi2b antibodies MX-35 (v18992) and lifastuzumab (v18993) were included as comparators and the anti-RSV antibody palivizumab (v22277) was included as a negative control.

Briefly, HEK293-6e cells were transfected for ˜24 hours to transiently express human NaPi2b, cynomolgus or mouse NaPi2b, at 1 μg of DNA per 1 million cells. Following transfection, 50,000 cells per well were seeded in V-bottom 96-well plates and incubated with 200 nM of primary antibody for 18-24 hours at 4° C. to prevent internalization. Post-incubation, cells were washed and stained with anti-Human IgG Fc AF647 conjugate (Jackson Immuno Research Labs, West Grove, PA, Cat. No. 109-605-098, Lot. No. 124868) at 4° C. for 1 hour. Following staining and washing, fluorescence was detected by flow cytometry on BD LSRFortessa™ Cell Analyzer (BD Biosciences, Franklin Lake, NJ) with 10,000 minimum events collected per well. The AF647/APC-A GeoMean (fluorescence signal geometric mean, proportional to anti-Human AF647 binding) was calculated for the live singlet cell population for each primary antibody using FlowJo™ v8 software (BD Biosciences, Franklin Lake, NJ). The Bmax and Kd of each primary antibody were calculated using GraphPad Prism Version 9 (GraphPad Software, San Diego, CA)

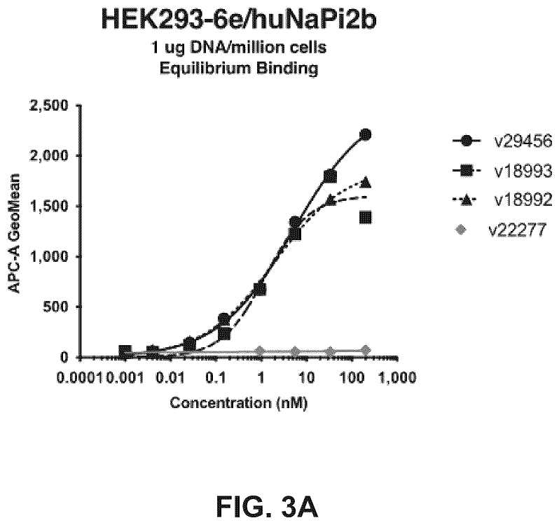

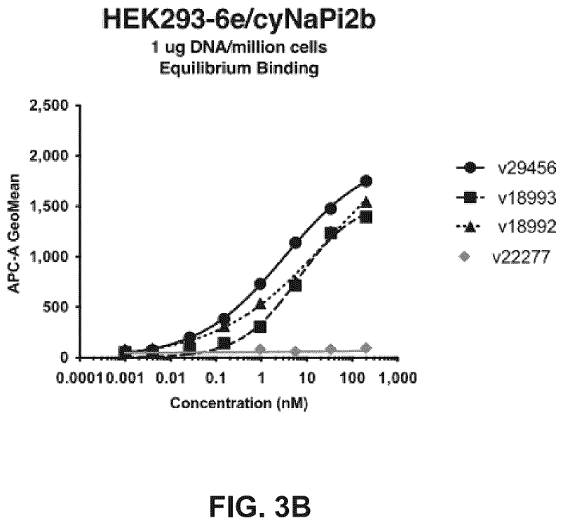

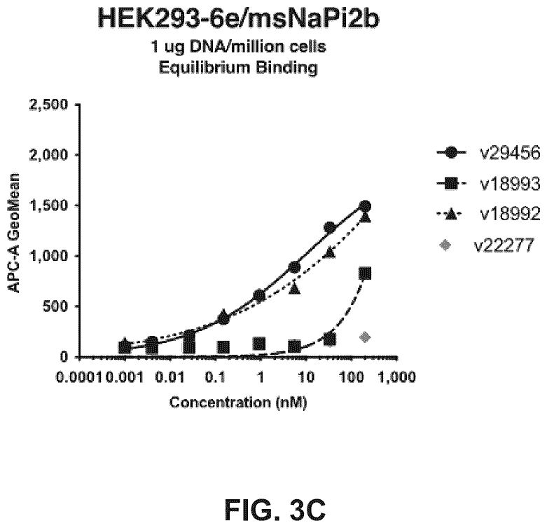

The binding results of humanized antibody variant v29456, MX-35 (v18992) and lifastuzumab (v18993) are shown in Table 5.1 and FIG. 3A (human NaPi2b), FIG. 3B (cynomolgus NaPi2b), and FIG. 3C (mouse NaPi2b).

TABLE 5.1
Cross-reactivity of v29456 to cynomolgus and mouse NaPi2b

| | Bmax (APC-A GeoMean) | | | Apparent Kd (nM) | | |
|---|---|---|---|---|---|---|
| | Hu | Cyno | Ms | Hu | Cyno | Ms |
| Sample | NaPi2b | NaPi2b | NaPi2b | NaPi2b | NaPi2b | NaPi2b |
| v29456 | 2,538 | 2,006 | 2,062 | 5.20 | 3.20 | 10.59 |
| v18992 | 1,896 | 2,365 | 3,930 | 1.98 | 31.03 | >200* |
| v18993 | 1,607 | 1,557 | N/A | 1.22 | 6.27 | N/A |
| v22277 | N/A | N/A | N/A | N/A | N/A | N/A |

*Apparent Kd value greater than the highest antibody testing concentration (>200 nM).

v29456 and v18992 showed binding to human, cynomolgus and mouse NaPi2b on transfected HEK296-6e cells. Lifastuzumab (v18993) displayed binding to human and cynomolgus NaPi2b and showed minimal binding to mouse NaPi2b transfected HEK293-6e cells.

v29456 had comparable apparent Kd value to v18992 and v18993 on human NaPi2b binding and exhibited the greatest binding to cynomolgus NaPi2b, yielding apparent Kd 10-fold and 2-fold lower than v18992 (MX35) and v18993 (lifastuzumab), respectively. Negative control palivizumab (v22277) did not bind to any species tested, as expected.

### Example 6: Functional Characterization of Anti-NaPi2b Antibody Constructs—Cell-Binding of Monovalent Antibodies by Kinexa

The binding affinity of anti-NaPi2b antibody constructs was assessed by Kinexa in the endogenous NaPi2b-expressing cell line, IGROV-1. Affinity of antibody constructs was assessed in monovalent format to reduce the effects of avidity and internalization and compared directly to the parental chimeric antibody variant, also in monovalent format. v29814 (monovalent format of parental chimeric variant 23855), v36123 (monovalent format of humanized antibody variant 29452, and v36124 (monovalent format of humanized antibody variant 29456) were assessed. The experiment was performed as described below.

### IGROV-1 Cell Preparation:

IGROV-1 cells were cultured in RPMI 1640 Medium, ATCC modification (Thermo Fisher Scientific, Waltham, MA) supplemented with 10% FBS (Thermo Fisher Scientific, Waltham, MA) in T175 culture flasks (Corning, Corning, NY) and incubated at 37° C. with 5% CO₂ until achieving 80% confluency. Cells were detached from culture vessels by incubation with Cell Dissociation Buffer (Invitrogen, Waltham, MA) for 30-60 minutes at 37° C. with 5% CO₂, collected by neutralizing Cell Dissociation Buffer with at least 5 times volume of RPMI 1640 Medium, ATCC modification supplemented with 10% FBS, and maintained on ice until use. Cells were counted using the Vi-Cell™ XR Cell Viability Analyzer (Beckman Coulter, Brea, California).

### KinExA Preparation:

The solid phase was prepared by coating one vial of PMMA (polymethyl methacrylate) beads (Sapidyne, Boise, Idaho) with 1 mL of 20 μg/mL BSA-biotin (Sigma-Aldrich, St. Louis, Missouri) in PBS pH 7.4. The beads were incubated for 2 hours at room temperature with gentle rotation. Beads were settled, supernatant removed, and beads rinsed five times with PBS pH 7.4. The beads were then coated with 1 mL of 100 μg/mL of streptavidin (Jackson ImmunoResearch, West Grove, PA) in PBS pH 7.4 with 10 mg/mL BSA (Sigma-Aldrich, St. Louis, Missouri) with rotation at room temperature for 1 hour. Beads were settled, supernatant removed, and beads rinsed five times with PBS pH7.4. The final step of the solid phase preparation was done by coating with 30 μg/mL of biotin goat anti-human IgG (Jackson ImmunoResearch, West Grove, PA) for 1 hour with rotation at room temperature.

### Cell-Binding Assay:

The cell-binding assay was set up using the antibody constructs or variants as constant binding partner at two different concentrations of 50 pM and 500 pM. For the titration curve with antibody variants fixed at 50 pM, at least 10 million IGROV-1 cells were used as titrant. For the titration curve with antibody variants fixed at 500 pM, at least 5 million cells were used as titrant. The antibody variants and cells were mixed in PBS pH 7.4, 1 mg/mL BSA, 0.2% NaN₃ and incubated at 4° C. for 7 days with gentle rotation until equilibrium was reached. After incubation, the mixture of antibody variants and cells was centrifuged to separate the cells from the unbound free antibody variants. The free antibody variants were loaded onto the KinExA 3200 (Sapidyne, Boise, Idaho) with biotinylated—anti-human IgG PMMA as solid phase and 0.5 μg/mL of Alexa 647 goat anti-human IgG (Jackson ImmunoResearch, West Grove, PA) as detection antibody.

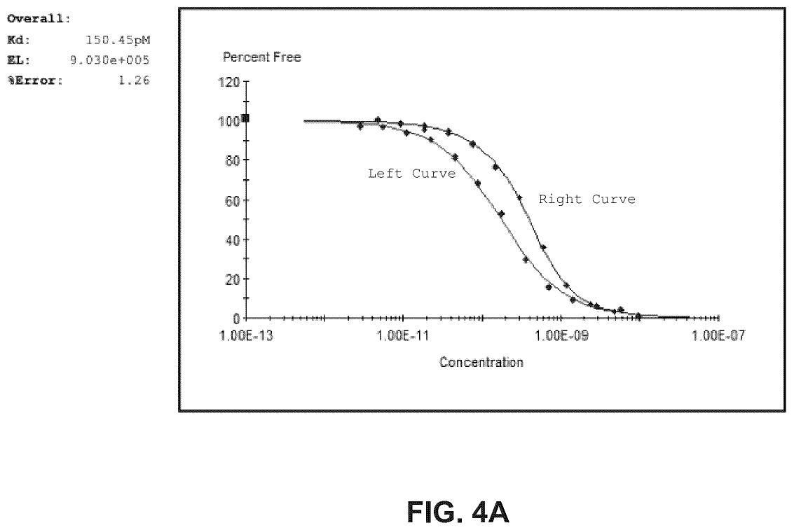

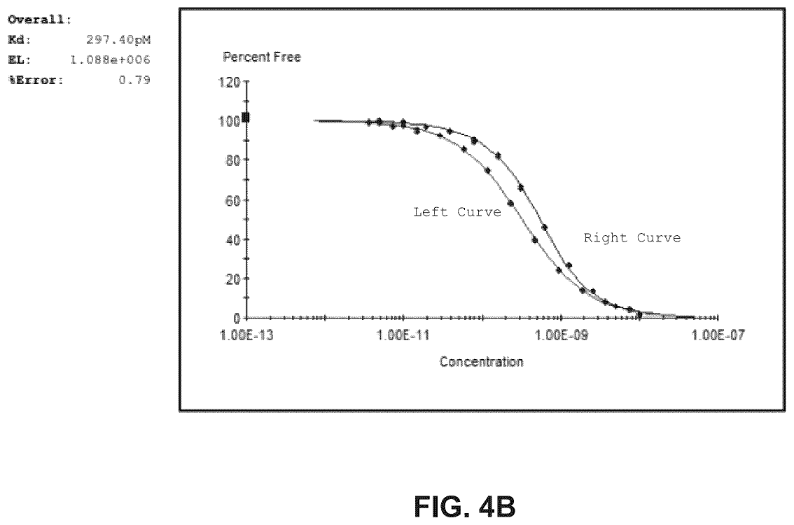

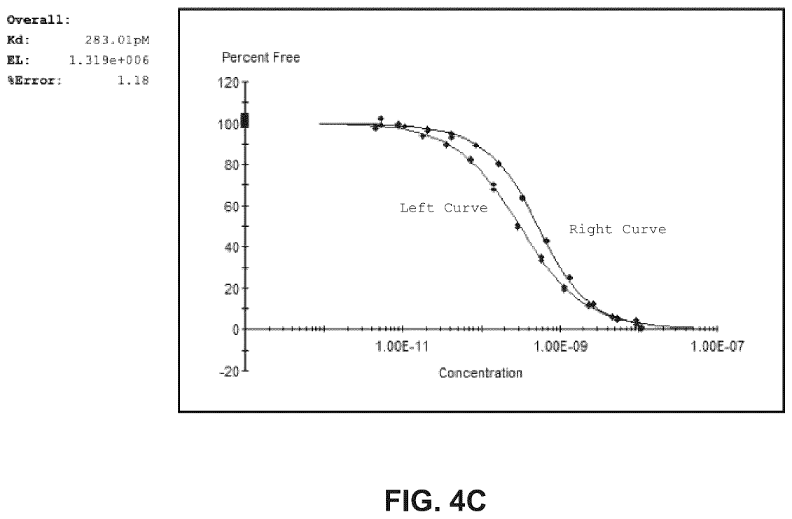

Results for parental chimeric monovalent antibody variant (v29814) and two humanized, monovalent antibody variants are shown in Table 6.1. N-curve analysis was used to calculate the affinity and receptor expression level with the concentrations of antibody variant as reference point. Narrow 95% confidence intervals were obtained for both the affinity and receptor expression level and % error of the fit was less than 1.5%. The N-curve analysis is shown in FIG. 4A (v29814), FIG. 4B (v36123), and FIG. 4C (v36124). For each panel, the right curve shows the data for 500 pM constant binding partner and the left curve shows the data for 50 pM constant binding partner.

TABLE 6.1
Binding of Anti-Napi2b Antibody Constructs to IGROV-1 cells

| | | Expression | |
|---|---|---|---|
| Variant/Construct | KD ^{#} | Level ^{#} | % Error |
| v29814 (parental | 150.45 pM | 9.01E+5 | 1.26 |
| chimeric) | (202.2 pM-114.3 pM) | (1.16E+6-7.34E+5) | |
| v36123 | 297.4 pM | 1.088E+6 | 0.79 |
| (VH1/VL3) | (379 pM-238.7 pM) | (1.37E+6-8.95E+5) | |
| v36124 | 283.01 pM | 1.32E+6 | 1.18 |
| (VH1/VL2) | (405.7 pM-207.8 pM) | (1.89E+6-1.0E+6) | |

^{#} Parentheses indicates 95% confidence interval

All humanized anti-NaPi2b antibody variants displayed similar binding profiles to each other and approximately 2-fold lower binding affinity compared to chimeric parental antibody construct. The calculated receptor expression level was between 0.9-1.3 million per cell.

### Example 7: Functional Characterization of Anti-NaPi2b Antibody Constructs—Internalization

Internalization of the chimeric parental anti-NaPi2b antibody v23855 and a representative humanized variant, v29456 (H1L2), in NaPi2b-expressing cell lines (HCC-78 and NCI-H441) was determined by flow cytometry as described below. The NaPi2b-targeting antibodies lifastuzumab (v18993) and MX35 (v18992) were used as positive controls, and the anti-RSV antibody palivizumab (v22277) was used as a negative control.

Briefly, antibodies were fluorescently labeled by coupling to a Fab fragment AF488 conjugate targeting anti-Human IgG Fe (Jackson Immuno Research Labs, West Grove, PA; Cat. No. 109-547-008) at a 1:1 molar ratio in PBS pH 7.4 (Thermo Fisher Scientific, Waltham, MA; Cat. No. 10010-023), for 24 hours at 4° C. Cells were seeded at 50,000 cells/well in RPMI 1640, ATCC modification (Thermo Fisher Scientific, Waltham, MA) supplemented with 10% fetal bovine serum (Thermo Fisher Scientific, Waltham, MA) in 48-well plates and incubated overnight under standard culturing conditions (37° C./5% CO₂) to allow attachment. Coupled antibodies were added to cells the following day at 10 nM and incubated under standard culturing conditions for 5-24 hours to allow for internalization. Following incubation, cells were dissociated, washed, and surface AF488 fluorescence was quenched using an anti-AF488 antibody (Life Technologies, Carlsbad, CA; Cat. No. A-11094) at 100 nM for 30 minutes at 4° C. Quenched AF488 fluorescence (internalized fluorescence) was detected by flow cytometry on a BD LSRFortessa™ Cell Analyzer (BD Biosciences, Franklin Lake, NJ) with 1,000 minimum events collected per well. The AF488/FITC-A GeoMean (fluorescence signal geometric mean, proportional to anti-Human Fab AF488 labelling) was calculated for the live single cell population using FlowJo™ Version 10.8.1 (BD Biosciences, Franklin Lake, NJ) and plotted using GraphPad Prism Version 9 (GraphPad Software, San Diego, CA).

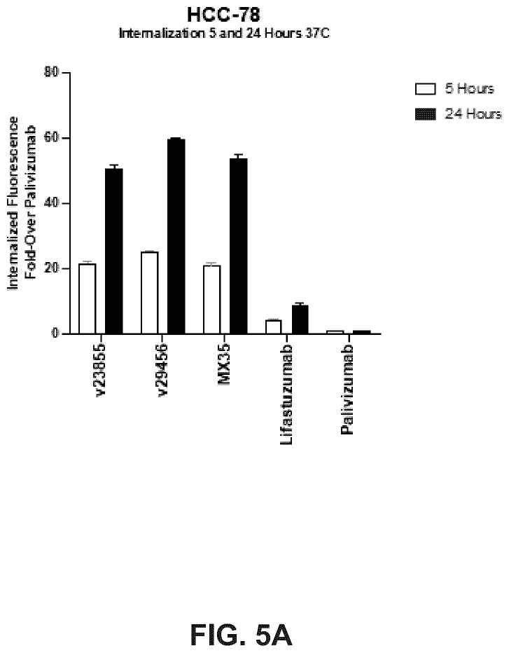

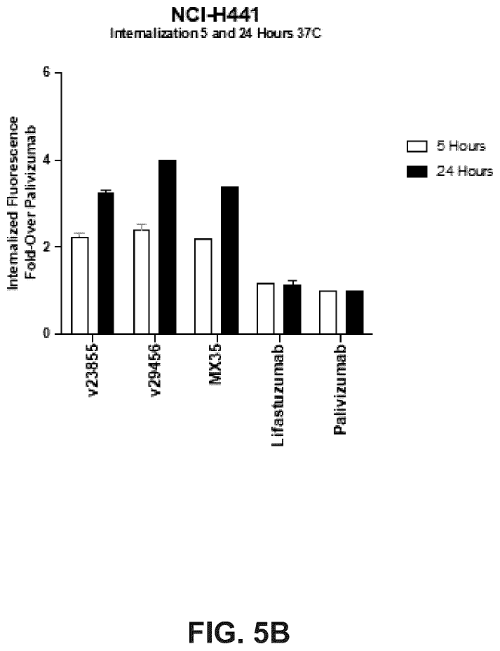

Results are shown in FIG. 5A (HCC-78) and FIG. 5B (NCI-H441), and in Table 7.1 below.

TABLE 7.1
Internalization of Antibody Constructs

| | Internalized Fluorescence Fold-Over Palivizumab | | | |
|---|---|---|---|---|
| | HCC-78 | | NCI-H441 | |
| Antibody (10 nM) | 5 hrs | 24 hrs | 5 hrs | 24 hrs |
| v23855 (parental | 21.9 | 50.9 | 2.2 | 3.3 |
| chimeric) | | | | |
| v29456 (H1L2) | 25.3 | 59.9 | 2.4 | 4.0 |
| v18992 (MX35) | 21.1 | 53.9 | 2.2 | 3.4 |
| v18993 | 4.6 | 9.1 | 1.2 | 1.2 |
| (lifastuzumab) | | | | |
| v22277 | 1.0 | 1.0 | 1.0 | 1.0 |
| (neg control) | | | | |

Chimeric parental antibody v23855 and the humanized antibody variant v29456 showed comparable levels of internalization to the humanized antibody MX35 (v18992) and much greater levels of internalization compared to humanized antibody lifastuzumab (v18993) across all time points (5 hours and 24 hours) at 10 nM antibody treatment on both HCC-78 and NCI-H441 cells. For instance, following a 5-hour incubation in HCC-78, v23855 and v29456 showed 21.9 and 25.3-fold increase in internalized fluorescence compared to negative control palivizumab, respectively. Similarly, following a 24-hour incubation in HCC-78 cells, v23855 and v29456 showed 50.9- and 59.9-fold increase in internalized fluorescence compared to negative control palivizumab, respectively.

### Example 8: Developability Assessment of Anti-NaPi2b Antibodies

The isoelectric point, propensity for self-aggregation, and non-specific binding of anti-NaPi2b antibodies v23855 (parental chimeric), v29452 (H1L3) and v29456 (H1L2), were determined in order to assess the developability of these antibodies. The isoelectric point was measured by capillary isoelectric focusing (cIEF), the propensity for self-aggregation was measured by Affinity-capture self-interaction nanoparticle spectroscopy (AC-SINS) and non-specific binding was measured by NS-ELISA, as described below.

Capillary Isoelectric Focusing (cIEF)

cIEF was carried out using Maurice C. (ProteinSimple®) system, System Suitability Kit and Method Development Kit. System suitability standard, fluorescence calibration standard, cartridge and samples were prepared according to vendor's recommendations. The capillary was automatically calibrated with a fluorescence standard preconditioned with Maurice cIEF System Suitability Kit to ensure the capillary was functioning properly. The antibody samples were diluted to a concentration of 0.5 mg/mL in a final volume of 40 μL in Gibco™ Distilled Water, and mixed Maurice cIEF Method Development Kit Samples. The samples were then vortexed, centrifuged and the supernatant pipetted into individual wells of a 96-well plate. All electropherograms were detected with UV absorbance at 280 nm. All data analyses were performed using vendor software Compass for iCE (ProteinSimple®). The Compass software aligns each electropherogram using the pI markers so that the x-axis is displayed as a normalized pI for each injection.

### AC-SINS Assay

AC-SINS method was carried out in a 384-well plate format (Corning®#3702). Initially, 20 nm gold nanoparticles (Ted Pella, Inc., #15705) washed with 0.22 μm filtered Gibco™ Distilled Water were coated with a mixture of capture antibody—80% AffiniPure Goat Anti-Human IgG (H+L) (Jackson ImmunoResearch Laboratories® #109-005-088), and the non-capture antibody −20% ChromPure Goat IgG, whole molecule (Jackson ImmunoResearch Laboratories® #005-000-003), that were initially buffer exchanged into 20 mM sodium acetate pH 4.3 and diluted to 0.4 mg/mL. The mixture of gold nanoparticles, capture antibody and non-capture antibody was incubated in the dark for 18 h at room temperature. Sites unoccupied on the gold nanoparticles were blocked with 1 μM thiolated polyethylene glycol (2 kD) in 20 mM sodium acetate, pH 4.3 to a final concentration of 0.1 μM, followed by 1 h incubation at room temperature. The coated nanoparticles were then concentrated by centrifugation at 21,000×g for 7 min, at 8° C. 95% of the supernatant was removed and the gold pellet was resuspended in the remaining buffer. 5 μL of concentrated nanoparticles were added to 45 μL of antibody at 0.05 mg/mL in Gibco™ PBS pH 7.4 in a 384-well plate. The coated nanoparticles were incubated with the antibody of interest for 4 h at room temperature in the dark. The absorbance was read from 450-700 nm at 1 nm increments, and a Microsoft Excel macro was used to identify the max absorbance, smooth the data, and fit the data using a second-order polynomial. The Alambda (nm) was calculated based on the smoothed max absorbance of the average blank (PBS alone) subtracted from the smoothed max absorbance of the antibody sample to determine the antibody AC-SINS score. Antibody-antibody interactions directly correlate with the shift in maximum absorbance wavelength of gold nanoparticles coated with the antibody of interest. The cutoff of Alambda 10 nm was set as high self-aggregation propensity of the antibody.

### NS-ELISA

NS-ELISA was used to measure the propensity of the antibodies to bind to a range of biomolecules to emulate the undesirable non-specific interactions to biological matrices in vivo as described below.

NS-ELISA was carried out in Corning®96-well EIA/RIA Easy Wash™ Clear Flat Bottom Polystyrene High Bind Microplate coated overnight at 4° C. with 50 μL of Heparin (Sigma, H3149) diluted with 50 mM sodium carbonate pH 9.6 to a final concentration of 250 μg/mL. The plate was incubated for 2 days at room temperature, wells that were coated with heparin were not covered to allow air dry. Insulin (Sigma-Aldrich®, 19278) and KLH (Sigma-Aldrich®, H8283) were each diluted with 50 mM sodium carbonate pH 9.6 to a final concentration of 5 μg/mL. ssDNA (Sigma-Aldrich®, D8899) and dsDNA (Sigma-Aldrich®, D4553) was diluted with Gibco™ PBS pH7.4 to a final concentration of 10 μg/mL. 50 μL each of insulin, KLH, dsDNA and ssDNA were added to a 96 well plate, followed by the incubation at 37° C. for 2 h. The coating materials were removed, and the plate was blocked with 200 μL of Gibco™ PBS pH7.4, 0.1% Tween®20, and incubated for 1 h at room temperature with shaking at 200 rpm. The plate was washed 3 times with Gibco™ PBS pH7.4, 0.1% Tween 20. 50 μL of each mAb at 100 nM (15 mg/mL) in Gibco™ PBS pH 7.4, 0.1% Tween®20 was added in duplicate to the wells and incubated for 1 h at room temperature with shaking at 200 rpm. Plates were washed three times with Gibco™ PBS pH7.4, 0.1% Tween 20, and 50 μL of 50 ng/mL anti-human IgG HRP (Thermofisher Scientific®, H10307) was added to each well. Plates were incubated for 1 h at room temperature, with shaking at 200 rpm. The plate was washed three times with Gibco™ PBS pH7.4, 0.1% Tween 20, and 100 μL of TMB substrate (Cell Signaling Technology®, 7004P6) added to each well. Reactions were stopped after approximately 10 minutes by adding 100 μL of 1 M HCl to each well, and absorbance was read at 450 nm. Binding scores were calculated as the ratio of the ELISA signal of the antibody (antibody-treated) to the signal of a well containing buffer instead of the primary antibody (untreated). The cutoffs considered for each binding molecule (ssDNA, KLH, insulin, dsDNA and heparin) were internally calculated, based on the average of Zymeworks Inc. produced antibodies and antibodies benchmarks published in the literature.

The results of all three assays are shown in Table 8.1. In these assays a score higher than the cutoff was taken to indicate potentially less desirable biophysical characteristics.

TABLE 8.1
Developability assessment results for anti-NaPi2b antibodies

| | | AC-SINS | NS-ELISA binding score (ratio of signal | | | | |
|---|---|---|---|---|---|---|---|
| | cIEF | Δlambda | between antibody-treated and untreated) | | | | |
| Sample | pI | (nm) | ssDNA | KLH | Insulin | dsDNA | Heparin |
| v23855 | 8.35 | 2.5 | 1.59 | 2.38 | 1.23 | 1.42 | 1.27 |
| v29452 | 8.53 | 2.0 | 1.22 | 1.34 | 1.03 | 1.0 | 1.05 |
| v29456 | 8.53 | 2.5 | 1.06 | 1.37 | 1.05 | 1.07 | 1.06 |

The pI values determined for the main isoform for the variants v23855; v29452 and v29456 are 8.35, 8.53 and 8.53 respectively, which all fall within the typical range for therapeutic antibodies. The analysis of Alambda showed no potential issues on AC-SINS for all variants. Additionally, there were no potential issues identified for NS-ELISA.

### Example 9: Stability of a Humanized Antibody Variant in Mouse Plasma or in PBS

The objective of this experiment was to assess if fragmentation of antibody variant v29456 and reference antibody MX35 (v18992) occurred over time after incubation in mouse plasma or in PBS pH 7.4 at 37° C.

Briefly, v29456 or v18992 were each diluted into either PBS or mouse plasma to a final concentration of 0.5 mg/ml and incubated at 37° C. Samples were removed after 0, 7 and 14 days and stored at −80° C. until characterization. For characterization, samples were thawed at room temperature and 50 μg were incubated with 5 μg of recombinant EndoS endoglycosidase for one hour at room temperature. An immunoprecipitation slurry was generated by a 45 min incubation of 95 μL/sample of magnetic Sepharose streptavidin-coated beads with 15 μg/sample of biotinylated goat anti-Human IgG Fc capture antibody, followed by 4 washes with PBS pH 7.4 with the aid of a DynaMag™-2 magnet (Invitrogen™).

After deglycosylation, plasma-incubated samples were incubated with 95 μL of the immunoprecipitation slurry for 1.5 hrs at room temperature. Then, the slurry underwent 6 washes with PBS pH 7.4 and 2 washes with LC-MS-grade water with the aid of a DynaMag™-2 magnet (Invitrogen™). A final wash with PBS pH 7.4 was performed before elution. Protein was eluted by incubating beads with 35 μL of LC-MS grade water with 20% acetonitrile and 0.1% formic acid for one hour at room temperature. PBS samples were not immunoprecipitated.

5 μL of the eluted sample were transferred to glass inserts in LC-MS vials. For LC-MS analysis, 1 μL of sample were injected into a Waters™ BioSuite Phenyl Column, 1000 Å, 10 μm, 4.6 mm×75 mm using a Waters™ ACQUITY™ UPLC I-Class HPLC system coupled to a Waters™ Synapt™ G2-Si HDMS with a column temperature of 70 C and a flow rate of 0.3 mL/min. Mobile phases consisted of A: LC-MS grade water with 0.1% v/v formic acid, 0.025 v/v trifluoroacetic acid and 10% v/v isopropyl alcohol in, and B: acetonitrile with 0.1% v/v formic acid and 10% v/v isopropyl alcohol. The column was pre-equilibrated in 10% mobile phase B before injection. Then, a 20 min 10 to 27% mobile phase B gradient was applied, followed by a 2 min 27 to 90% mobile phase B gradient and a column wash of 2 min at 99% mobile phase B.

The column was re-equilibrated to 10% mobile phase B for 2 minutes between runs. ESI was performed in positive mode with 3 kV of capillary voltage, 120 C source temperature, 100 V sampling cone voltage, source offset of 80 V, source gas flow of 0 ml/min, desolvation temperature 500 C, cone gas flow 0 μL/Hr, desolvation gas flow 800 μL/hr, nebuliser gas flow 6.5 bar. Data format was continuum with analyser set in sensitivity mode, with a m/z range from 500 to 7000.

Peak integration, MS deconvolution and mass assignments were performed in Protein Metrics Byos® v4.0 using a deconvolution window of 60000-160000 Da with an m/z range of 1000-4000. For all time points, the highest intensity deconvoluted mass was assigned as the reference mass of v29456. The reference mass was defined as the average mass of v29456 or v18992 with two 2-acetamido-2-deoxy-beta-D-glucopyranose-(1-4)-[alpha-L-fucopyranose-(1-6)] stubs arising from EndoS activity on N-glycans, 16 disulfide bonds and the formation of pyroglutamic acid, if applicable, at the N-terminus. Mass assignments had a mass tolerance of ±10 Da. Other assigned mAb proteoforms were: mAb reference mass with a phosphoric acid adduct, mAb reference mass with the loss of one fucose unit, mAb reference mass with the addition of a hexose unit. A pentasaccharide adduct (likely penta-mannose) relative to the reference mass of v29456 without 2-acetamido-2-deoxy-beta-D-glucopyranose-(1-4)-[alpha-L-fucopyranose-(1-6)] was also identified. Apparent purity was calculated as the ratio of all v29456 or v18992 mAb proteoforms deconvoluted peak intensities divided by all observed deconvoluted peak intensities. Mouse plasma proteins present in the day 0 mouse plasma control, but not observed in the PBS controls, were not considered in the apparent purity calculations.

The data is provided in Table 9.1 and shows that there was no evidence of fragmentation of v29456 incubated at 37° C. for 7 and 14 days in mouse plasma or PBS pH 7.4, as apparent purity after 7 and 14 days was similar to day 0 controls. v18992 showed fragmentation after 14 days in PBS based on appearance of low molecular weight species and a decrease in apparent purity of >10% relative to day 0 control. v18992 fragmentation was not observed in plasma.

TABLE 9.1
Stability of anti-NaPi2b antibodies in mouse
plasma (apparent % desired proteoforms)

| | v29456 | | v18992 | |
|---|---|---|---|---|
| Timepoint | PBS | Mouse Plasma | PBS | Mouse Plasma |
| (days) | 37° C. | 37° C. | 37° C. | 37° C. |
| 0 | 99.8% | 99.9% | 95.5% | 96.0% |
| 7 | 99.2% | 100.0% | 95.6% | 92.3% |
| 14 | 99.6% | 98.4% | 84.3% | 98.6% |

### Example 10: Quantification of Surface NaPi2b on Tumor Cells

Surface NaPi2b protein was measured on tumor cell lines by quantitative flow cytometry using a set of beads with known levels of antibody binding capacity (ABC) as described below. Reference humanized antibody MX35 (v18992) conjugated to Alexa Fluor® AF647 and control anti-RSV antibody palivizumab (v21995) conjugated to Alexa Fluor® AF647 were used to fluorescently label tumor cells and beads. Variant 22277 differs from anti-RSV antibody v21995 used in previous examples in that it has a heterodimeric Fc. This does not affect the function of this antibody. The representative cell lines evaluated were OVCAR-3, IGROV-1, HCC-78, TOV-21G, NCI-H441, HCT116, and EBC-1.

Conjugations of v18992 and v21995 to Alexa Fluor® AF647 were performed as follows: v18992 and v21995 were each reacted with 8 equivalents of NHS-AF647 (Thermo Fisher #A20006, 10 mM) in PBS. The reaction was protected from light at room temperature, and was allowed to proceed for 200 and 150 minutes, respectively. Following incubation, the reactions were purified through four and two rounds of purification, respectively, using a 40 kDa Zeba column (Thermo Fisher, Waltham, MA) pre-equilibrated with PBS, pH 7.4. Confirmation of conjugation and quantification of unconjugated NHS-AF647 were measured by SEC chromatography (Ex: 650 nm, Em: 665 nm).

Cells were detached from culture vessels using Cell Dissociation Buffer (Invitrogen, Waltham, MA) and seeded at 50,000 cells/well in conical-bottom 96-well plates in triplicate. Cells and anti-Human QSC® beads (Bangs Laboratories, Inc., Fishers, IN) were stained with v18992-AF647 with a pre-determined excess level of conjugated antibody or negative control v21995-AF647 at the same concentration and incubated for 30 minutes at 4° C. Following incubation, cells and beads were washed in FACS buffer and analyzed on the BD™ Fortessa HTS and processed using FlowJo™ v8 software (BD Biosciences, Franklin Lake, NJ).

The median AF647 fluorescence intensity for all bead populations were plotted against relevant ABC values using Bangs Laboratories QuickCal v 2.3 calibration line template for QSC® anti-Human IgG Lot #14490.

Surface protein expression for cell lines was calculated based on a monovalent binding model and therefore equivalent to the background subtracted ABC (SABC). The median fluorescence intensity of v21995-AF647-stained cells from each of the respective cell lines was used as a background value for determination of SABC.

Results are shown in Table 10.1. Reported NaPi2b proteins per cell are an average of at least two biological replicates. Tumor cell lines were designated with high, mid, low, or negative expression of the target; cell lines were designated as “high” expressors if the average number of NaPi2b proteins detected was greater than 900,000 per cell; “mid” if the number was between 40,000 and 900,000 per cell; “low” if the number was between 500 and 40,000 per cell; and “negative” if the number was negative (below limit of quantitation of the calibration beads).

TABLE 10.1
Surface NaPi2b quantification on tumor cell lines

| | Tissue and | Average | | |
|---|---|---|---|---|
| Cell Line | Disease | NaPi2b/cell | % CV* | Designation |
| OVCAR-3 | Ovarian | 2,340,000 | 18% | High |
| | adenocarcinoma | | | |
| IGROV-1 | Ovarian | 1,770,000 | 41% | High |
| | carcinoma | | | |
| HCC-78 | Lung carcinoma | 970,000 | 48% | High |
| | (NSCLC) | | | |
| TOV-21G | Ovarian | 442,000 | 51% | Mid |
| | adenocarcinoma | | | |
| NCI-H441 | Lung | 40,000 | 9% | Mid |
| | adenocarcinoma | | | |
| HCT-116 | Colorectal | 1,270 | 183% | Low |
| | carcinoma | | | |
| EBC-1 | Lung carcinoma | <0 | −333% | Negative |
| | (NSCLC) | | | |

*Percent coefficient of variation calculated across two to seven biological replicate measurements

### Example 11: Preparation of Antibody-Drug Conjugates

Antibody-drug conjugates (ADCs) shown in Table 11.1 were prepared. Exemplary protocols for preparing these ADCs are provided below, followed by a description of the Drug-Linkers (DLs) in Table 11.2 and structures of the payloads.

TABLE 11.1
Antibody-drug conjugates and DAR

| Variant | Antibody | Drug-Linker | DAR |
|---|---|---|---|
| v29456 | Humanized | MC-GGFG-AM-DXd1 (DL1) | 8 |
| | antibody | MT-VC-Compound 1 (DL2) | 4 |
| | variant | | |
| v23855 | Parental | MT-VC-Compound 1 (DL2) | 4 |
| | Chimeric | MC-VC-PABC-MMAE (DL3) | 4 |
| v18992 | MX35 | MC-GGFG-AM-DXd1 (DL1) | 8 |
| | (reference | MT-VC-Compound 1 (DL2) | 3 |
| | antibody) | | |
| v18993 | Lifastuzumab | MC-GGFG-AM-DXd1 (DL1) | 8 |
| | (reference | MT-VC-Compound 1 (DL2) | 4 |
| | antibody) | MC-VC-PABC-MMAE (DL3) | 4 |
| v22277 | Palivizumab | MC-GGFG-AM-DXd1 (DL1) | 8 |
| | control | MT-VC-Compound 1 (DL2) | 4 |
| | | TFP-AD-VC-Compound 1 (DL4) | 4 |
| | | MC-VC-PABC-MMAE (DL3) | 4 |
| | | | 8 |

### Exemplary Protocols

v29456-MC-GGFG-AM-DXd1 DAR8: A solution (2.47 mL) of the humanized variant v29456 (54 mg) in PBS, pH 7.4 was diluted in PBS, pH 7.4 (1.00 mL) and reduced by addition of 5 mM diethylenetriamine pentaacetic acid (DTPA) (0.90 mL in PBS, pH adjusted to 7.4) and 25 mM of an aqueous tris(2-carboxyethyl)phosphine (TCEP) solution (134 μL, 9.0 eq.). After 4 hours at 37° C., the reduced antibody was purified by passage over a Zeba™ Spin Desalting Columns (40 KDa MWCO; Thermo Scientific™) pre-equilibrated with 1 mM DTPA in PBS, pH 7.4. An aliquot of the reduced antibody solution (13.5 mg, 1.32 mL) was diluted with 1 mM DTPA (33.5 μL in PBS, pH adjusted to 7.4). To the antibody solution was added 38.3 μL of DMSO and an excess of MC-GGFG-AM-DXd1 (111.7 μL; 12 eq.) from a 10 mM DMSO stock solution. The conjugation reaction proceeded at room temperature with mixing for 120 min. At that time, additional drug-linker (14 μL, 1.5 eq.) was added. The conjugation reaction proceeded at room temperature with mixing for an additional 60 min. An excess of a 10 mM N-acetyl-L-cysteine solution (76.8 μL, 8 eq.) was added to quench the conjugation reaction.

v29456-MT-VC-Compound 1 DAR4: A solution (43.9 μL) of the humanized variant v29456 (0.5 mg) in PBS, pH 7.4 was diluted in PBS, pH 7.4 (44.3 μL) and reduced by addition of 5 mM diethylenetriamine pentaacetic acid (DTPA) (24 μL in PBS, pH adjusted to 7.4) and 1 mM of an aqueous tris(2-carboxyethyl)phosphine (TCEP) solution (7.76 μL, 2.25 eq.). After 2 hours at 37° C., the reduced antibody was cooled down to 0-4° C. An excess of MT-VC-Compound 1 (1.72 μL, 10 eq.) from a 20 mM stock solution was added. The conjugation reaction proceeded at 0-4° C. for 90 min.

v23855-MT-VC-Compound 1 and v23855-MC-VC-PABC-MMAE: A solution (2.52 mL) of the chimeric variant v23855 (15 mg) in PBS, pH 7.4 was diluted in PBS, pH 7.4 (457 μL) and reduced by addition of 5 mM diethylenetriamine pentaacetic acid (DTPA) (750 μL in PBS, pH adjusted to 7.4) and 10 mM of an aqueous tris(2-carboxyethyl)phosphine (TCEP) solution (26.0 μL, 2.6 eq.). After 2 hours at 37° C., the reduced antibody was cooled down to 0-4° C. An excess of MT-VC-Compound 1 or MC-VC-PABC-MMAE (50.0 μL, 10 eq.) from a 20 mM stock solution was added. The conjugation reaction proceeded at 0-4° C. for 60 min. An excess of a 10 mM N-acetyl-L-cysteine solution (26.7 μL, 8 eq.) was added to quench the conjugation reaction. The quenching reaction proceeded at 0-4° C. for 30 min.

v18992-MC-GGFG-AM-DXd1 DAR8: A solution (120.8 μL) of the humanized variant v18992 (1 mg) in PBS, pH 7.4 was diluted in PBS, pH 7.4 (29.6 μL) and reduced by addition of 5 mM diethylenetriamine pentaacetic acid (DTPA) (40.0 μL in PBS, pH adjusted to 7.4) and 10 mM of an aqueous tris(2-carboxyethyl)phosphine (TCEP) solution (9.62 μL, 14.0 eq.). After 3 hours at 37° C., the reduced antibody was purified by passage over a Zeba™ Spin Desalting Columns (40 KDa MWCO; Thermo Scientific™) pre-equilibrated with 10 mM NaOAc, pH 4.5. To the reduced antibody solution was added 20.0 μL of DMSO and an excess of MC-GGFG-AM-DXd1 (10.31 μL; 15 eq.) from a 10 mM DMSO stock solution. The conjugation reaction proceeded at room temperature with mixing for 120 min.

v18992-MT-VC-Compound 1 DAR3: A solution (4.23 mL) of the humanized variant v18992 (35 mg) in PBS, pH 7.4 was diluted in PBS, pH 7.4 (2.2 mL) and reduced by addition of 5 mM diethylenetriamine pentaacetic acid (DTPA) (1.75 mL in PBS, pH adjusted to 6.7) and 10 mM of an aqueous tris(2-carboxyethyl)phosphine (TCEP) solution (50.8 μL, 2.1 eq.). After 2 hours at 37° C., the reduced antibody was cooled down to 0-4° C. An excess of MT-VC-Compound 1 (120.9 μL, 10 eq.) from a 20 mM stock solution was added. The conjugation reaction proceeded at 0-4° C. for 60 min. An excess of a 10 mM N-acetyl-L-cysteine solution (64.5 μL, 8 eq.) was added to quench the conjugation reaction. The quenching reaction proceeded at 0-4° C. for 30 min.

v18993-MC-GGFG-AM-DXd1 DAR8: A solution (156.5 μL) of the humanized variant v18993 (1 mg) in PBS, pH 7.4 was diluted in PBS, pH 7.4 (1.96 μL) and reduced by addition of 5 mM diethylenetriamine pentaacetic acid (DTPA) (42.0 μL in PBS, pH adjusted to 7.4) and 10 mM of an aqueous tris(2-carboxyethyl)phosphine (TCEP) solution (9.55 μL, 14.0 eq.). After 3 hours at 37° C., the reduced antibody was purified by passage over a Zeba™ Spin Desalting Columns (40 KDa MWCO; Thermo Scientific™) pre-equilibrated with 10 mM NaOAc, pH 4.5. To the reduced antibody solution was added 20.0 μL of DMSO and an excess of MC-GGFG-AM-DXd1 (10.23 μL; 15 eq.) from a 10 mM DMSO stock solution. The conjugation reaction proceeded at room temperature with mixing for 120 min.

v18993-MT-VC-Compound 1 DAR4: A solution (78.3 μL) of the humanized variant v18993 (0.5 mg) in PBS, pH 7.4 was diluted in PBS, pH 7.4 (10.25 μL) and reduced by addition of 5 mM diethylenetriamine pentaacetic acid (DTPA) (24.0 μL in PBS, pH adjusted to 7.4) and 1 mM of an aqueous tris(2-carboxyethyl)phosphine (TCEP) solution (7.50 μL, 2.2 eq.). After 2 hours at 37° C., the reduced antibody was cooled down to 0-4° C. An excess of MT-VC-Compound 1 (1.71 μL, 10 eq.) from a 20 mM stock solution was added. The conjugation reaction proceeded at room temperature for 60 min.

v18993-MC-VC-PABC-MMAE DAR4: A solution (5.84 mL) of the humanized variant v18993 (30 mg) in PBS, pH 7.4 was diluted in PBS, pH 7.4 (0.60 mL) and reduced by addition of 5 mM diethylenetriamine pentaacetic acid (DTPA) (1.62 mL in PBS, pH adjusted to 7.4) and 10 mM of an aqueous tris(2-carboxyethyl)phosphine (TCEP) solution (55.3 μL, 2.7 eq.). After 2.5 hours at 37° C., the reduced antibody was cooled down to 0-4° C. PBS, pH 7.4 (0.24 mL) was added, followed by an excess of MC-VC-PABC-MMAE (225 μL, 11 eq.) from a 10 mM stock solution was added. The conjugation reaction proceeded at 0-4° C. for 16 h. An excess of a 10 mM N-acetyl-L-cysteine solution (0.90 mL, 44 eq.) was added to quench the conjugation reaction. The quenching reaction proceeded at room temperature for 60 min.

v22277-MC-GGFG-AM-DXd1 DAR8: A solution (2.18 mL) of the control variant v22277 (10 mg) in PBS, pH 7.4 was diluted in PBS, pH 7.4 (4.1 μL) and reduced by addition of 5 mM diethylenetriamine pentaacetic acid (DTPA) (563 μL in PBS, pH adjusted to 6.7) and 10 mM of an aqueous tris(2-carboxyethyl)phosphine (TCEP) solution (68.9 μL, 10.0 eq.). After 3 hours at 37° C., the reduced antibody was purified by passage over a Zeba™ Spin Desalting Columns (40 KDa MWCO; Thermo Scientific™) pre-equilibrated with 10 mM NaOAc, pH 5.5. To the reduced antibody solution was added 230.0 μL of DMSO and an excess of MC-GGFG-AM-DXd1 (51.7 μL; 15 eq.) from a 20 mM DMSO stock solution. The conjugation reaction proceeded at room temperature with mixing for 60 min. An excess of a 20 mM N-acetyl-L-cysteine solution (51.7 μL, 15 eq.) was added to quench the conjugation reaction. The quenching reaction proceeded at 0-4° C. for 30 min.

v22277-MT-VC-Compound 1 DAR4, v22277-MC-VC-PABC-MMAE DAR4: A solution (4.36 mL) of the control variant v22277 (20 mg) in PBS, pH 7.4 was reduced by addition of 5 mM diethylenetriamine pentaacetic acid (DTPA) (1.10 mL in PBS, pH adjusted to 7.4) and 10 mM of an aqueous tris(2-carboxyethyl)phosphine (TCEP) solution (35.1 μL, 2.55 eq.). After 1.5 hours at 37° C., the reduced antibody was cooled down to 0-4° C. An excess of MT-VC-Compound 1 or MC-VC-PABC-MMAE (82.7 μL, 12 eq.) from a 20 mM stock solution was added. The conjugation reaction proceeded at room temperature or at 0-4° C. for 60 min.

v22277-AD-VC-Compound 1 DAR4: A solution (4.31 mL) of the variant v22277 (20 mg) was diluted with 0.69 mL of PBS, pH 7.4. An excess of TFP-AD-VC-Compound 1 (65.4 μL, 9.5 eq.) from a 20 mM DMSO stock solution was added, and conjugation proceeded at room temperature overnight.

v22277-MC-VC-PABC-MMAE DAR8: A solution (1.08 mL) of the control variant v22277 (5 mg) in PBS, pH 7.4 was diluted in PBS, pH 7.4 (81.1 μL) and reduced by addition of 5 mM diethylenetriamine pentaacetic acid (DTPA) (300 μL in PBS, pH adjusted to 6.7) and 10 mM of an aqueous tris(2-carboxyethyl)phosphine (TCEP) solution (41.3 μL, 10.0 eq.). After 3 hours at 37° C., the reduced antibody was purified by passage over a Zeba™ Spin Desalting Columns (40 KDa MWCO; Thermo Scientific™) pre-equilibrated with PBS, pH 7.4. To the reduced antibody solution was added an excess of MC-VC-PABC-MMAE (20.7 μL; 12 eq.) from a 20 mM DMSO stock solution. The conjugation reaction proceeded at room temperature with mixing for 60 min.

TABLE 11.2
Drug-Linkers used in the preparation of ADCs

| Drug-linker¹ | Linker | Payload |
|---|---|---|
| DL1 | MC-GGFG-AM- | DXd1 |
| DL2 | MT-VC- | Compound 1 |
| DL3 | MC-VC-PABC- | MMAE |
| DL4 | AD-VC | Compound 1 |

¹Structures of Linkers and Payloads provided below

### Structures:

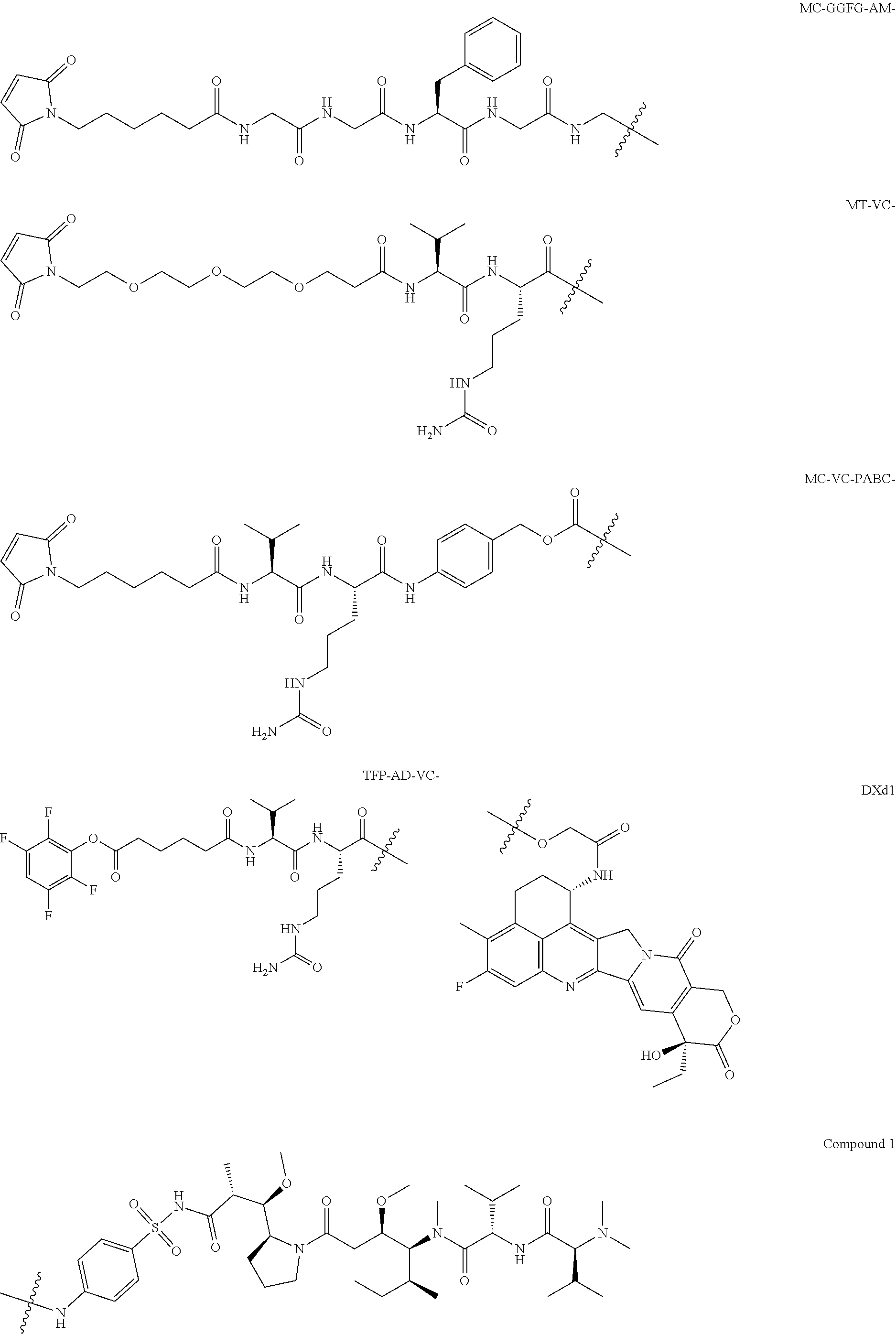

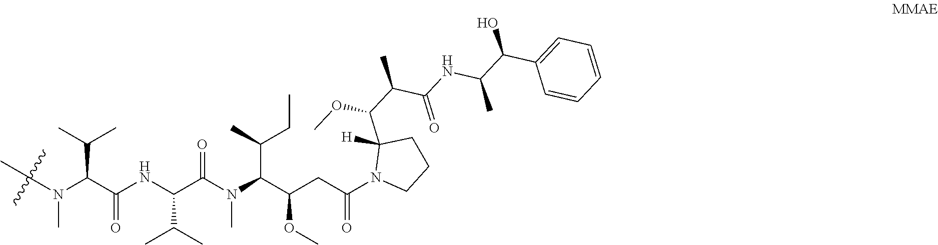 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

### Example 12: Purification and Characterization of Antibody-Drug Conjugates

ADCs prepared through Cysteine conjugation chemistry as described in Example 11 were purified by one to two passages over a Zeba™ Spin Desalting Columns (40 KDa MWCO; Thermo Scientific™) pre-equilibrated with 10 mM NaOAc, pH 4.5 or pH 5.5, or with PBS, pH 7.4.

ADCs prepared at a large scale as described in Example 11 were purified on an AKTA™ pure chromatography system (Cytiva Life Sciences, Marlborough, MA) using a 53 mL HiPrep 26/10 Desalting column (Cytiva Life Sciences, Marlborough, MA) and a mobile phase consisting of 10 mM NaOAc, pH 4.5 with 150 mM NaCl and a flow rate of 10 mL/min. The purified ADC was then sterile filtered (0.2 m).

ADCs prepared through lysine conjugation chemistry as described in Example 11 were purified by one passage over a Zeba™ Spin Desalting Columns (40 KDa MWCO; Thermo Scientific™) pre-equilibrated with PBS, pH 7.4.

Following purification, ADC prepared for in vivo use were then sterile filtered (0.2 mm).

Following purification, the concentration of the ADCs was determined by a BCA assay with reference to a standard curve generated using the humanized variant v29456. Alternatively, concentrations were estimated by measurement of absorption at 280 nm using extinction coefficients taken from the literature (European Patent No. 3 342 785, for DL1) or determined experimentally (for the remaining drug-linkers). ADCs were also characterized by hydrophobic interaction chromatography (HIC), Reverse-Phase Ultra Performance Liquid Chromatography Mass Spectrometry (RP-UPLC-MS), and size exclusion chromatography (SEC) as described below.

### Hydrophobic Interaction Chromatography

Antibody and ADCs were analyzed by HIC to estimate the drug-to-antibody ratio (DAR). Chromatography was performed on an Agilent Infinity II 1290 HPLC (Agilent Technologies, Santa Clara, CA) using a TSKgel® Butyl-NPR column (2.5 μm, 4.6×35 mm; TOSOH Bioscience GmbH, Griesheim, Germany) and employing a gradient of 95/5% MPA/MPB to 5/95% MPA/MPB over a period of 12 minutes at a flow rate of 0.5 mL/min (MPA=1.5 M (NH₄)₂SO₄, 25 mM NaₓPO₄, pH 7 and MPB=75% 25 mM NaₓPO₄, pH 7, 25% isopropanol). Detection was by absorbance at 280 nm.

### Reverse-Phase Ultra Performance Liquid Chromatography Mass Spectrometry:

Alternatively, ADCs could be analyzed by RP-UPLC-MS to determine the average drug-to-antibody ratio (DAR).

### DAR Determination by RP-UPLC-MS for Lysine-Conjugated ADCs

ADC samples were deglycosylated using Endo S for 1 hour at RT and injected onto an Agilent 1290 Infinity II LC coupled with an Agilent 6545 Quadrupole Time of Flight (Q-TOF) mass spectrometer (Agilent Technologies, Santa Clara, CA). Protein species were separated using a PLRP-S column (1000 Å, 8 μM, 50×2.1 mm) at a flow rate of 0.3 ml/min using the gradient shown in Table 12.1. Mobile phase A: 0.1% formic acid (FA), 0.025% trifluoracetic acid (TFA) and 10% isopropyl alcohol (IPA) in water. Mobile phase B: 0.1% FA and 10% IPA in acetonitrile (ACN).

TABLE 12.1
RP-HPLC-MS Gradient

| Time (min) | % Buffer A | % Buffer B |
|---|---|---|
| 0 | 80 | 20 |
| 20 | 60 | 40 |
| 22 | 10 | 90 |
| 22.5 | 1 | 99 |
| | 1 | 99 |

The MS source conditions are shown in Table 12.2 and the acquisition parameters were as follows:

Mode: MS; Mass Range: 500 to 7000 m/z; Acquisition Rate: 1 spectra/s and 1000 ms/spectrum, 3354 transients/spectrum.

TABLE 12.2
MS Source Conditions

| | |
|---|---|
| Gas Temp: 300° C. | VCap: 5000 V |
| Drying Gas: 13 l/min | Nozzle Voltage: 2000 V |
| Nebulizer: 45 psig | Fragmentor: 170 V |
| Sheath Gas Temp: 400° C. | Skimmer: 65 V |
| Sheath Gas Flow: 12 l/min | Oct RF Vpp: 750 V |

Qualitive analysis using MassHunter software (Agilent Technologies, Santa Clara, CA) was employed for deconvolution and data analysis. Deconvolution parameters were as follows: Deconvolution Algorithm: Maximum Entropy; Mass Range: 70000-160000; Mass Step: 1.0; Used limited m/z range: 1000-7000; Subtract baseline: 7.0; Adduct: Proton; Isotope width: Automatic; Height Filter: Peak signal to noise >=30.0; Maximum number of peaks: Limited by height 100.

Average DAR was the calculated from the deconvoluted spectrum using the following equation:

$$average DAR = {\sum }_{i = 0}^{n} \frac{Drug {load}_{i} \cdot peak {intensity}_{i}}{Total peak intensity}$$

### DAR Determination by RP-UPLC-MS for Cysteine-Conjugated ADCs

ADC samples were deglycosylated using Endo S for 1 hour at RT, reduced and denatured by TCEP incubation at 70° C. for 1 h and injected onto an Agilent 1290 Infinity II LC coupled with an Agilent 6545 Quadrupole Time of Flight (Q-TOF) mass spectrometer (Agilent Technologies, Santa Clara, CA). Protein species were separated using a PLRP-S column (1000 Å, 8 μM, 50×2.1 mm) at a flow rate of 0.3 ml/min using the gradient shown in Table 12.3. Mobile phase A: 0.1% formic acid (FA), 0.025% trifluoracetic acid (TFA) and 10% isopropyl alcohol (IPA) in water. Mobile phase B: 0.1% FA and 10% IPA in acetonitrile (ACN).

TABLE 12.3
RP-HPLC-MS Gradient

| Time (min) | % Buffer A | % Buffer B |
|---|---|---|
| 0 | 80 | 20 |
| 20 | 60 | 40 |
| 22 | 10 | 90 |
| 22.5 | 1 | 99 |
| 24 | 1 | 99 |

The MS source conditions are shown in Table 12.4 and the acquisition parameters were as follows: Mode: MS; Mass Range: 500 to 7000 m/z; Acquisition Rate: 1 spectra/s and 1000 ms/spectrum, 3354 transients/spectrum.

TABLE 12.4
MS Source Conditions

| | |
|---|---|
| Gas Temp: 300° C. | VCap: 5000 V |
| Drying Gas: 13 l/min | Nozzle Voltage: 2000 V |
| Nebulizer: 45 psig | Fragmentor: 170 V |
| Sheath Gas Temp: 400° C. | Skimmer: 65 V |
| Sheath Gas Flow: 12 l/min | Oct RF Vpp: 750 V |

Qualitive analysis using MassHunter software (Agilent Technologies, Santa Clara, CA) was employed for deconvolution and data analysis. For a typical IgG1 ADC, the reduction of the sample should separate out the different species, light chain (LC) eluting first (DAR 0 followed by DAR 1, 2), heavy chain (HC) eluting later (DAR 0 followed by DAR 1, 2, 3). TIC is integrated at two regions corresponding to light and heavy chains and then deconvoluted. Deconvolution parameters were as follows:

Deconvolution Algorithm: Maximum Entropy; Mass Range: 20000-60000; Mass Step: 1.0; Used limited m/z range: 700-3000; Subtract baseline: 7.0; Adduct: Proton; Isotope width: Automatic; Height Filter: Peak signal to noise >=30.0; Maximum number of peaks: Limited by height 100.

Average DAR was the calculated from the deconvoluted spectrum using the following equation:

$$average DAR = 2 * ( {\sum }_{i = 0}^{n} \frac{LC peak intensityi * Drug loadi}{Total LC intensity} + {\sum }_{i = 0}^{n} \frac{HC peak intensityi * Drug loadi}{Total HC intensity} )$$

### Size Exclusion Chromatography

The extent of aggregation of the antibody and ADCs (˜15-150 g, 5 μL injection volume) was assessed by SEC on an Agilent Infinity II 1260 HPLC (Agilent Technologies, Santa Clara, CA) using an AdvanceBio SEC column (300 angstroms, 2.7 μm, 7.8×150 mm) (Agilent, Santa Clara, California) and a mobile phase consisting of 150 mM phosphate, pH 6.95 and a flow rate of 1 mL/min. Detection was by absorbance at 280 nm.

### Results

When determining the DAR by HIC, the individual contributions of the DAR0, DAR2, DAR4, DAR6 and DAR8 species to the average DAR of the purified ADCs were assessed by integration of the HPLC-HIC chromatogram. The average drug to antibody ratio (DAR) of each ADC was determined by the weighted average of each DAR species. Regardless of the method, the average DAR for each ADC, when rounded to the nearest integer, was the same as the target DAR shown in Table 12.1 below.

The extent of aggregation and monomer content was assessed by integration of the HPLC-SEC chromatogram. The monomer peak of each ADC was identified as the peak with the same retention time as the unconjugated antibody from which each ADC was derived from. All peaks with an earlier retention time relative to the monomer species was determined to be aggregated species. Percent monomer species determined for each ADC is shown in Table 12.1.

TABLE 12.1
Characterization of ADCs

| | Drug- | Target | % |
|---|---|---|---|
| Variant | Linker1 | DAR | Monomer |
| v29456 | DL1 | 8 | 88 |
| | DL2 | 4 | 99 |
| v23855 | DL2 | 4 | 99 |
| | DL3 | 4 | 99 |
| v18992 | DL1 | 8 | 88 |
| | DL2 | 3 | 99 |
| v18993 | DL1 | 8 | 88 |
| | DL2 | 4 | 98.3 |
| | DL3 | 4 | 100 |
| v22277 | DL1 | 8 | 98.5 |
| | DL2 | 4 | 100 |
| | DL4 | 4 | 100 |
| | DL3 | 4 | 99 |
| | | 8 | 100 |

### Example 13: Functional Characterization of Anti-NaPi2b Antibody Constructs—Cellular Binding of Bivalent Antibodies by Flow Cytometry

The ability of the parental chimeric antibody construct v23855 and the humanized antibody variants described in Example 2 and Example 3 to bind to NaPi2b expressed on cells was assessed on the endogenous NaPi2b-expressing cell line, IGROV-1, by flow cytometry. IGROV-1 cells express endogenous NaPi2b at a high level.

Briefly, cells were seeded at 50,000 cells/well in conical-bottom 96-well plates and treated with test antibody for 24 hours at 4° C. to prevent internalization. Palivizumab (anti-RSV antibody, v22277) was included as a negative control. Reference anti-NaPi2b antibodies lifastuzumab (v18993) and MX35 (v18992) conjugated to a maleimide functionalized auristatin drug linker (DL2) were included as comparators; conjugation of this drug linker has shown no impact to the antibody binding capability (data not shown). Following incubation, cells were washed and stained with anti-Human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. The AF647/APC-A GeoMean (fluorescence signal geometric mean, proportional to anti-Human AF647 binding) was calculated for the live cell population using FlowJo™ Version 10.8.1 (BD Biosciences, Franklin Lake, NJ) and plotted for each test antibody using GraphPad Prism Version 9 (GraphPad Software, San Diego, CA).

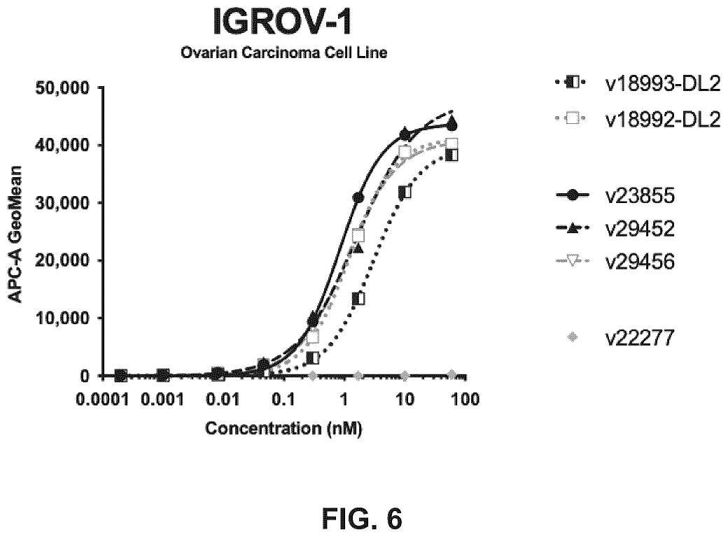

Results for parental chimeric construct (v23855) and all humanized antibody variants are tabulated in Table 13.1. Full dose-response binding curves are represented for parental chimeric antibody (v23855) and two representative humanized antibody variants (v29452, v29456) in FIG. 6.

TABLE 13.1
Binding of parental chimeric and humanized
antibody variants to IGROV-1 cells

| | IGROV-1 | | |
|---|---|---|---|
| | Bmax | Kd | Curve Hill |
| Test Antibody | (MFI) | (nM) | Slope (h) |
| v23855 | 43,706 | 0.84 | 1.2 |
| (parental chimeric) | | | |
| v18993-DL2 | 39,758 | 3.00 | 1.1 |
| (Lifa Compound 1 ADC) | | | |
| v18992-DL2 | 41,234 | 1.22 | 1.2 |
| (MX35 Compound 1 ADC) | | | |
| v29449 (H4L3) | 44,460 | 0.79 | 1.3 |
| v29450 (H3L3) | 43,809 | 0.96 | 1.2 |
| v29451 (H2L3) | 46,170 | 1.49 | 1.1 |
| v29452 (H1L3) | 47,763 | 1.60 | 0.9 |
| v29453 (H4L2) | 44,480 | 0.82 | 1.2 |
| v29454 (H3L2) | 42,278 | 0.92 | 1.0 |
| v29455 (H2L2) | 41,917 | 1.18 | 1.1 |
| v29456 (H1L2) | 40,867 | 1.10 | 1.1 |
| v29457 (H4L1) | 44,539 | 1.73 | 1.0 |
| v29458 (H3L1) | 40,793 | 1.39 | 1.1 |
| v29459 (H2L1) | 40,941 | 1.61 | 1.2 |
| v29460 (H1L1) | 40,071 | 1.67 | 1.2 |
| v22277 | NB | NB | NB |
| (Palivizumab) | | | |

All humanized antibody variants bound to IGROV-1 cells similarly, yielding apparent Kd values within 2-fold and comparable Bmax values. Reference antibody MX35-DL2 ADC showed comparable binding to chimeric v23855 and humanized antibodies. Humanized antibody lifastuzumab-DL2 ADC showed lower binding compared to all other targeted antibodies, with a lower Bmax value and greater apparent Kd value. Negative control palivizumab (v22277) showed no cellular binding (NB), as expected.

### Example 14: Functional Characterization of Anti-NaPi2b ADCS—Cellular Binding by Flow Cytometry

The ability of ADCs to bind to cells expressing NaPi2b was assessed. ADCs of a representative humanized variant antibody, v29456 (H1L2), were prepared by conjugation to DL2 and DL1 as described in Example 11. Binding to endogenous high NaPi2b-expressing cell lines IGROV-1 and HCC-78 was assessed by flow cytometry according to methods described in Example 5.

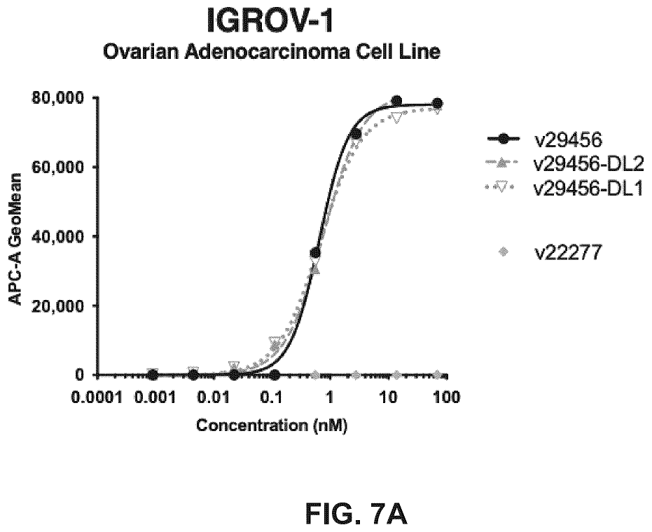

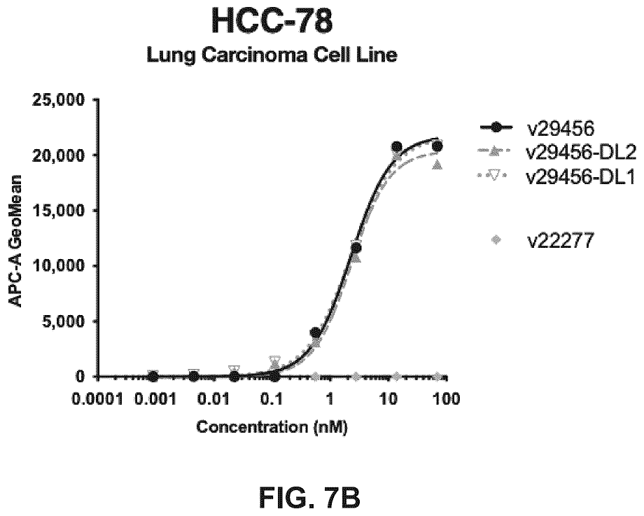

Results are shown in Table 14.1 and plotted in FIG. 7A for IGROV-1 cells and in FIG. 7B for HCC-78 cells.

TABLE 14.1
Cellular Binding of Antibody and Antibody-Drug Conjugates

| | IGROV-1 | | | HCC-78 | | |
|---|---|---|---|---|---|---|
| | Bmax | Kd | Curve Hill | | Kd | Curve Hill |
| Sample | (MFI) | (nM) | Slope (h) | Bmax | (nM) | Slope (h) |
| v29456 | 78,059 | 0.65 | 1.7 | 21,785 | 2.27 | 1.2 |
| v29456-DL2 | 81,406 | 0.77 | 1.3 | 20,399 | 2.32 | 1.3 |
| v29456-DL1 | 77,049 | 0.67 | 1.2 | 21,759 | 2.27 | 1.1 |
| v22277 | NA | NA | NA | NA | NA | NA |
| (Palivizumab) | | | | | | |

Both antibody drug conjugates yielded similar apparent Kd and Bmax values compared to their unconjugated parental antibody v29456 on both high NaPi2b-expressing cell lines IGROV-1 and HCC-78. Negative control Palivizumab (v22277) did not bind to either cell line, as expected.

These results indicate that the ability of the representative antibody variant v29456 to bind to cells expressing NaPi2b was not affected by conjugation to drug-linkers.

### Example 15: Functional Characterization of Anti-NaPi2b ADCS—In Vitro Cytotoxicity in 2D Monolayer Culture

The cytotoxicity as measured by cell growth inhibition of the humanized variant v29456 (H1L2) conjugated to various drug-linkers were assessed in a panel of NaPi2b-expressing cell lines as described below. Cell lines used were OVCAR-3 (ovarian carcinoma), IGROV-1 (ovarian carcinoma), and HCC-78 (lung carcinoma). ADCs with antibody palivizumab (anti-RSV) (v22277) were used as non-targeted controls.

OVCAR-3 were cultured in RPMI 1640 Medium, ATCC modification (Thermo Fisher Scientific, Waltham, MA) supplemented with 20% fetal bovine serum (FBS) (Thermo Fisher Scientific, Waltham, MA) and 0.01 μg/mL human insulin (Sigma-Aldrich, Oakville, ON). IGROV-1 and HCC-78 were cultured in RPMI 1640 Medium, ATCC modification (Thermo Fisher Scientific, Waltham, MA) supplemented with 10% FBS (Thermo Fisher Scientific, Waltham, MA). Briefly, cells were seeded in 384-well plates at 50 μL per well of their standard culture medium and treated with 20 μL per well of a titration of test article prepared in RPMI 1640 Medium, ATCC modification (Thermo Fisher Scientific, Waltham, MA) supplemented with 10% FBS. Cells were incubated for 4 days under standard culturing conditions. After incubation, CellTiter-Glo® reagent (Promega Corporation, Madison, WI) was added in all wells and luminescence corresponding to ATP present in each well was measured using a Synergy™H1 plate reader (BioTek Instruments, Winooski, VT). Percent cytotoxicity values were calculated by the following formula: (1−(Luminescence of Treated Cells/Average Luminescence of Untreated Cells))×100, and plotted against test article concentration using GraphPad Prism 9 software (GraphPad Software, San Diego, CA). EC50 values were calculated based on a non-linear regression log(agonist) versus response, variable slope (four parameters) by GraphPad Prism 9.

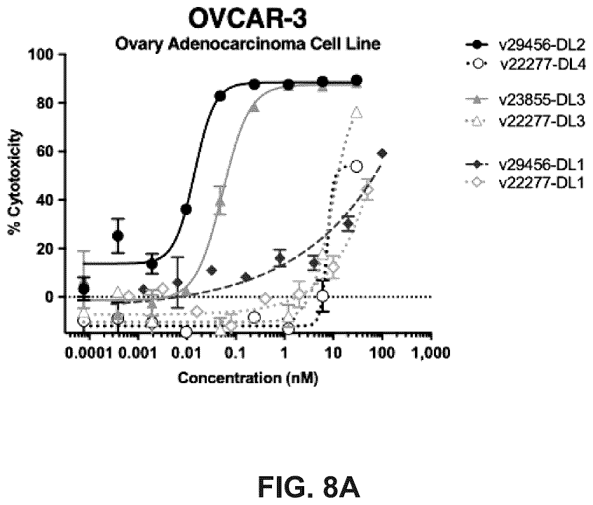

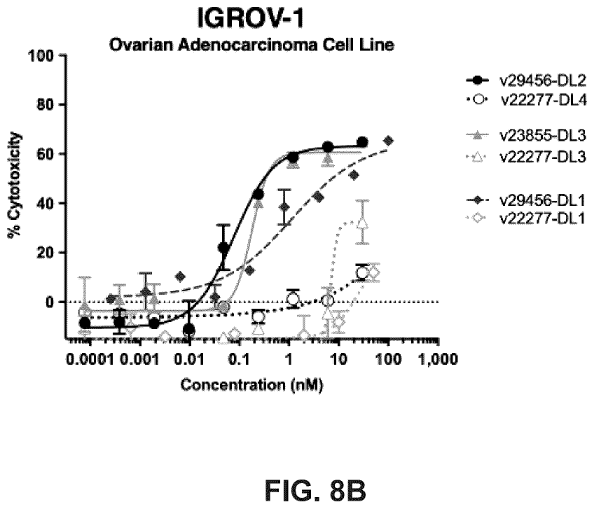

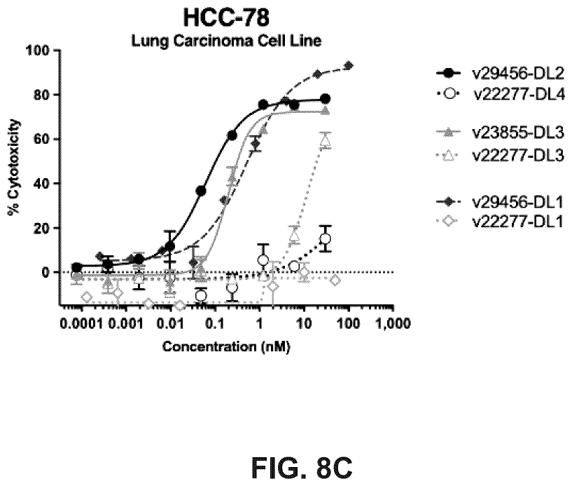

The results are shown in Table 15.1 and representative curves are plotted in FIG. 8A (OVCAR-3), FIG. 8B (IGROV-1), and FIG. 8C (HCC-78).

TABLE 15.1
In vitro Cytotoxicity - 2D Monolayer

| | | EC50 (nM) | | |
|---|---|---|---|---|
| ADC | DAR | OVCAR-3 | IGROV-1 | HCC-78 |
| v29456-DL2 | 4 | 0.01 | 0.08 | 0.06 |
| v18993-DL2 | 4 | 0.49 | 2.12 | 1.77 |
| v18992-DL2 | 3 | 0.02 | 0.11 | 0.13 |
| v22277-DL4 | 4 | >30 | >30 | >30 |
| v29456-DL1 | 8 | IC* | 1.14 | 0.51 |
| v18993-DL1 | 8 | IC* | 3.45 | 5.45 |
| v18992-DL1 | 8 | IC* | 1.04 | 0.68 |
| v22277-DL1 | 8 | >50 | >50 | >50 |
| v23855-DL3 | 4 | 0.05 | 0.18 | 0.20 |
| v22277-DL3 | 4 | 9.40 | 7.02 | 11.43 |

*Incomplete Curve

The humanized variant v29456 and humanized antibody MX35 (v18992) showed comparable potency to each other and greater potency than humanized antibody lifastuzumab (v18993) when comparing conjugates containing the same drug linker (DL2 and MC-GGFG-DXd) against all three NaPi2b-expressing cell lines. EC50 values for NaPi2b-targeted killing could not be calculated for DXd ADCs in OVCAR-3 cells (incomplete curve). The chimeric antibody v23855 conjugated to DL3 showed NaPi2b-targeted killing with sub-nanomolar potency against the three cell lines. As expected, palivizumab control ADCs showed over log-fold greater EC50 values than NaPi2b-targeted ADCs against all three NaPi2b-expressing cell lines.

### Example 16: In Vivo Activity of Chimeric Antibody Drug Conjugates

In vivo anti-tumor activity of a chimeric v23855 ADC was assessed in the OVCAR3 xenograft model of ovarian cancer, which expresses high levels of NaPi2b. The anti-tumor activities of ADCs based on reference antibodies lifastuzumab (v18993) and MX35 (v18992) were also assessed for comparison. The studies were carried out as described below.

These data indicate v23855 based ADCs are active against NaPi2b expressing tumors in vivo.

For the high Napi2b expressing OVCAR3 ovarian cancer model, tumor fragments (˜1 mm³) from stock mice were implanted subcutaneously into female CB.17 SCID mice. When mean tumor volume reached ˜100-150 mm³ the animals were assigned to groups, n=8 per group, and administered a single IV dose of test articles on study day 1. Tumor volume and body weight were measured twice weekly with a study duration of 39-49 days. For statistical analyses, a linear mixed effects model was fit to log-transformed tumor volumes, followed by F-test for the null hypothesis that mean growth rates are equal and post-hoc pairwise comparisons. Two studies were carried out with treatment groups as described in Table 16.1 and Table 16.2 below.

TABLE 16.1
Treatment groups for OVCAR3 Study 1

| | | | Dose |
|---|---|---|---|
| Group | Treatment | DAR | (mg/kg) |
| 1 | Vehicle | | 0 |
| 2 | v23855-DL2 | 4 | 6 |
| 3 | v23855-DL2 | 4 | 18 |
| 4 | v18992-DL2 | 4 | 6 |
| 5 | v18992-DL2 | 4 | 18 |

TABLE 16.2
Treatment groups for OVCAR3 Study 2

| | | | Dose |
|---|---|---|---|
| Group | Treatment | DAR | (mg/kg) |
| 1 | Vehicle | | 0 |
| 2 | v23855-DL2 | 4 | 6 |
| 3 | v18993-DL2 | 4 | 6 |
| 4 | v23855-DL3 | 4 | 6 |
| 5 | v23855-DL3 | 4 | 18 |
| 6 | v18993-DL3 | 4 | 6 |

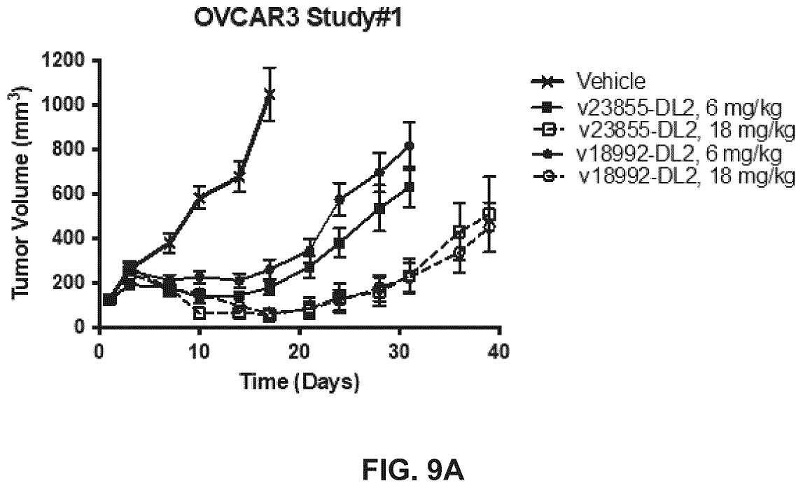

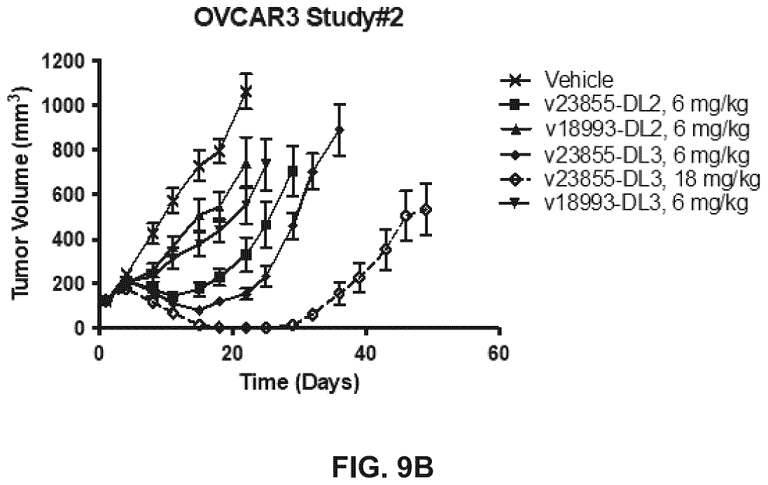

The results for OVCAR3 Study 1 are shown in FIG. 9A and demonstrate that 6 and 18 mg/kg doses of parental chimeric antibody v23855-DL2 resulted in significant inhibition of tumor growth compared to vehicle control (p<0.05). v23855-DL2 inhibited tumor growth that was comparable to reference antibody v18992-DL2 at either the 6 or 18 mg/kg dose levels.

The results for the OVCAR3 Study 2 are shown in FIG. 9B and demonstrate that a 6 mg/kg dose of chimeric v23855-DL2 resulted in a significant inhibition of tumor growth compared to vehicle control (p<0.05). At 6 mg/kg, v23855-DL2 resulted in a greater inhibition of tumor growth than reference antibody v18993-DL2. At 6 mg/kg, v23855-DL3 resulted in a greater inhibition of tumor growth than reference antibody v18993-DL3.

Together, these studies demonstrate that ADCs comprising the parental chimeric v23855 show superior anti-tumor activity to ADCs comprising the reference antibody v18993, when conjugated to either DL2 or DL3. When conjugated to DL2, v23855 showed non-inferior activity to the reference antibody v18992.

### Example 17: In Vivo Activity of Humanized Antibody Drug Conjugates

In vivo anti-tumor activity of an ADC of humanized antibody variant v29456 conjugated to DXd1 was assessed in the OVCAR3 xenograft model of ovarian cancer and in the NCI-H441 xenograft model of lung cancer, both models in which tumors express high levels of NaPi2b. The studies were carried out as described below.

For the high NaPi2b expressing OVCAR3 ovarian cancer model, tumor fragments (˜1 mm³) were implanted subcutaneously into female CB.17 SCID mice. When mean tumor volume reached ˜100-150 mm³ the animals were assigned to groups, n=8 per group, and administered a single IV dose of test articles on study day 1. Tumor volume and body weight were measured twice weekly with a study duration of 60 days. Treatment groups are described in Table 17.1.

TABLE 17.1
Treatment groups for OVCAR3 study

| | | | Dose |
|---|---|---|---|
| Group | Treatment | DAR | (mg/kg) |
| 1 | Vehicle | | 0 |
| 2 | v29456-DL1 | 8 | 1 |
| 3 | v29456-DL1 | 8 | 3 |
| 4 | v29456-DL1 | 8 | 10 |

For the NCI-H441 model of lung cancer, 5×106 cells in 0.1 ml 1:1 PBS:Matrigel were implanted subcutaneously into male NU-Foxn1nu mice. When mean tumor volume reached ˜145 mm³ the animals were assigned to groups, n=6 per group, and administered a single IV dose of test articles on study day 0. Tumor volume and body weight were measured twice weekly with a study duration of 28-35 days. For statistical analyses, a linear mixed effects model was fit to log-transformed tumor volumes, followed by F-test for the null hypothesis that mean growth rates are equal and post-hoc pairwise comparisons. Two studies were carried out in the NCI-H441 model with treatment groups described in Table 17.2 and Table 17.3.

TABLE 17.2
Treatment groups for NCI-H441 Study 1

| | | | Dose |
|---|---|---|---|
| Group | Treatment | DAR | (mg/kg) |
| 1 | Vehicle | | 0 |
| 2 | v29456-DL1 | 8 | 1 |
| 3 | v29456-DL1 | 8 | 3 |
| 4 | v29456-DL1 | 8 | 10 |

TABLE 17.3
Treatment groups for NCI-H441 Study 2

| | | | Dose |
|---|---|---|---|
| Group | Treatment | DAR | (mg/kg) |
| 1 | Vehicle | | 0 |
| 2 | v29456-DL1 | 8 | 0.3 |
| 3 | v29456-DL1 | 8 | 1 |
| 4 | 21995 | 8 | 1 |
| | (palivizumab)-DL1 | | |

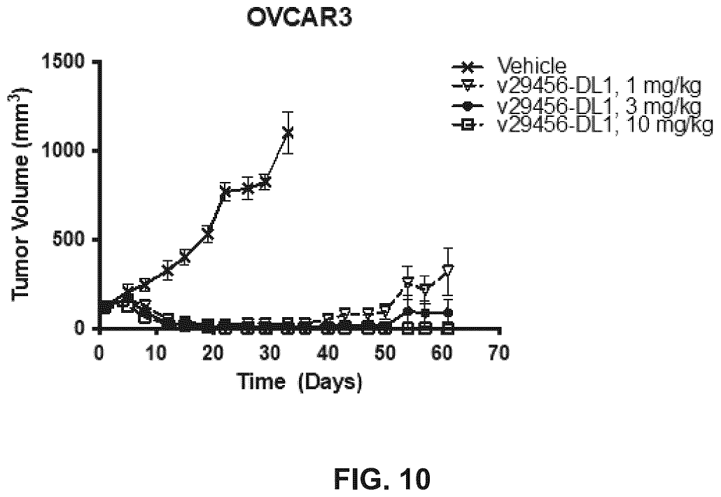

Results for the OVCAR3 model are shown in FIG. 10, showing that v29456-DL1 resulted in strong tumor growth inhibition at 1, 3 and 10 mg/kg.

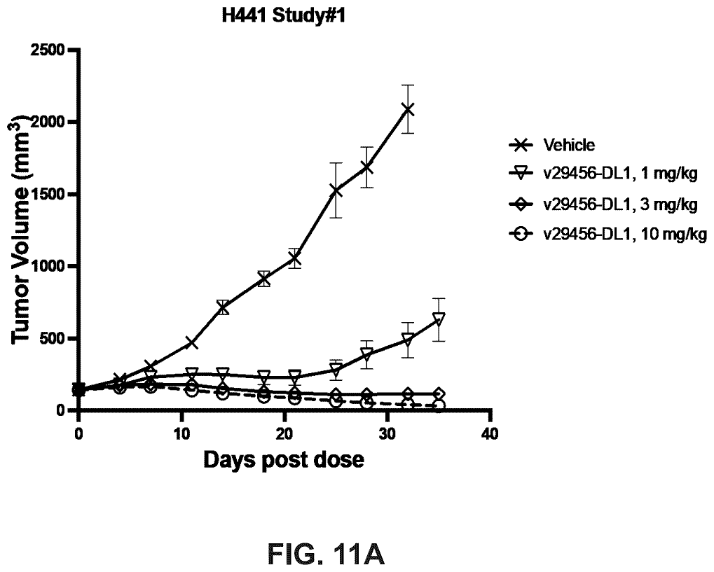

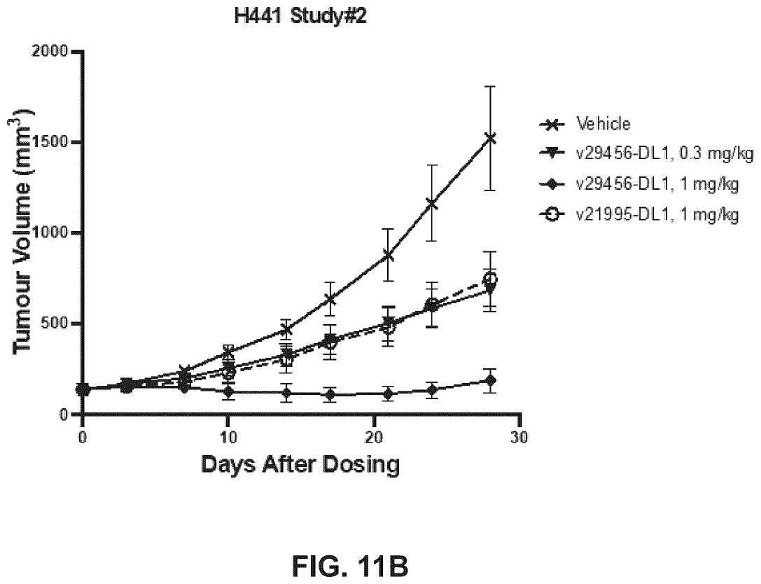

Results for the NCI-H441 model Study 1 are shown in FIG. 11A, showing that v29456-DL1 resulted in strong tumor growth inhibition at 1, 3 and 10 mg/kg.

The results of the NCI-H441 model Study 2 are shown in FIG. 11B and demonstrate that v29456-DL1 resulted in moderate inhibition of tumor growth at 0.3 mg/kg and strong tumor growth inhibition at 1 mg/kg. At 1 mg/kg, the activity of the non-targeting control v21995 (palivizumab)-DL1 was less than that of v29456-DL1, demonstrating the target dependent activity of the v29456 ADC.

### Example 18: Assessment of Specificity of Anti-NaPi2b Antibody

A Membrane Proteome Array™ (Integral Molecular, Philadelphia, PA, USA) was used to screen for specific off-target binding interactions for antibody, humanized v38591 anti-NaPi2b (SLC34A2) variant. This anti-NaPi2b humanized antibody variant has amino acid sequences that are essentially identical to v29456. The DNA sequence encoding the heavy chains of v38591 is identical to that encoding v29456, except that the heavy chains include a C-terminal lysine. This C-terminal lysine is cleaved from the majority of the antibody product once it is secreted from the cell it is produced in.

Briefly, the study consisted of three phases: phase (1) determination of assay screening conditions, phase (2) membrane proteome array (library) screen and phase (3) protein target validation. In phase (1), conditions appropriate for detecting v38591 binding by high-throughput flow cytometry were determined, including the optimal antibody concentration and cell type for screening (two cell types were tested, HEK293T and avian QT6). In phase (2), using optimal conditions determined in phase 1, v38591 was screened against the library of over 6000 human membrane proteins (individually expressed in unfixed HEK293T cells), including 94% of all single-pass, multi-pass, and GPI-anchored proteins, including GPCRs, ion channels and transporters. In phase (3), each protein target hit from the screen stage (potential off-target interactions) was assessed in titration experiment using flow cytometry.

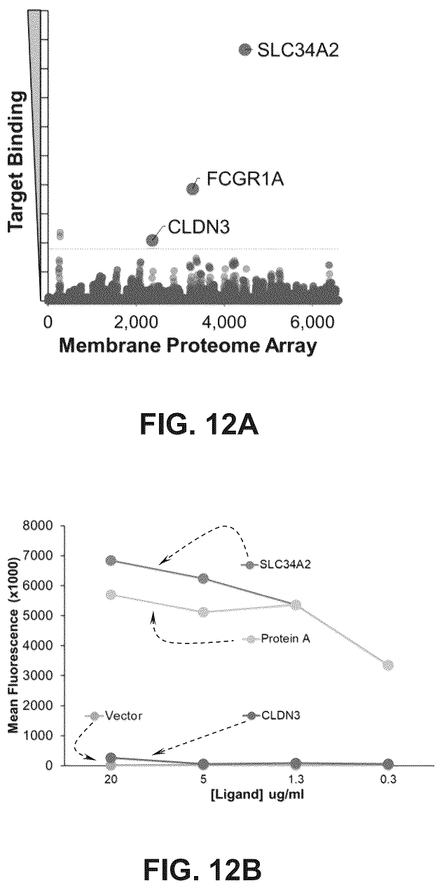

Phase (1) determined that the HEK293T cell type and an antibody concentration of 20 g/mL were optimal for library screening. As shown in FIG. 12A, library screening resulted in validated protein target hits for the primary target of NaPi2b as well as for FcgR1A which binds the Fc portion of the antibody. Another validated protein target hit was CLDN3. CLDN3 validation data indicated it was a very weak binder to humanized v38591, as shown by a low binding signal to v38591 across a range of concentrations (MFI-60-275), compared to a strong binding signal in the case of NaPi2b (MFI ˜3500-7000) in the validation assay (FIG. 12B). In general, this data is indicative of high specificity of humanized v38591 for the primary target, NaPi2b.

### Example 19: Functional Characterization of Anti-NaPi2B Antibody Cellular Binding of Anti-NaPi2B Antibody by Flow Cytometry

The ability of the humanized antibody variants v38591 and v29456 to bind to NaPi2b expressed on cells was assessed on endogenous NaPi2b-expressing tumor cell lines IGROV-1 (ovarian adenocarcinoma) and TOV-21G (ovarian carcinoma) by flow cytometry. IGROV-1 and TOV-21G cells express endogenous NaPi2b at high and moderate levels, respectively, as described in Example 10.

Cellular binding was performed according to methods described in Example 13. Reference anti-NaPi2b antibody lifastuzumab (v18993) and anti-RSV antibody palivizumab (v22277) were included as positive and negative controls, respectively.

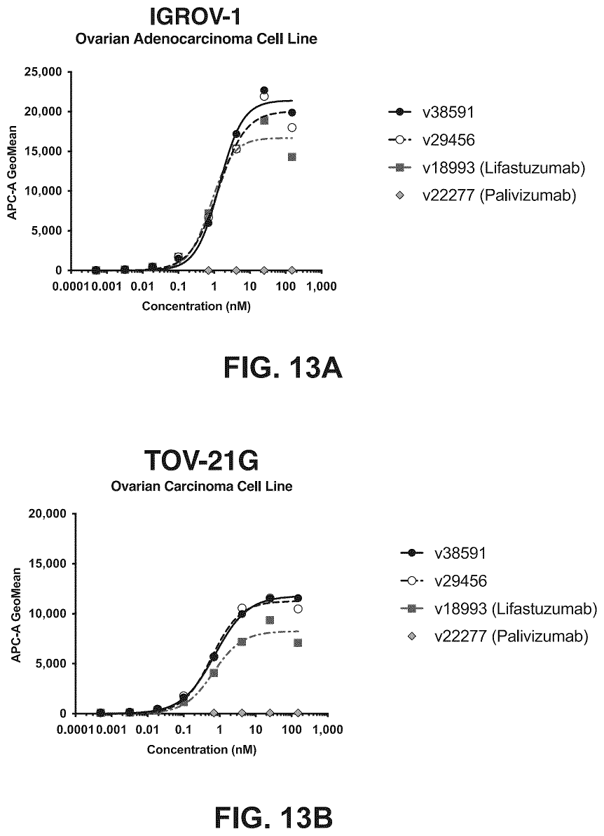

Results are shown in Table 19.1 and plotted in FIG. 13. Both v38591 and v29456 demonstrated comparable cellular binding to IGROV-1 and TOV-21G cell lines, indicating that addition of C-terminal lysine to the heavy chains of the antibody had minimal impact on the cellular binding capability of the antibody. Anti-NaPi2b antibody control v18993 showed slightly poorer binding with approximately 1.2- and 1.4-fold decreased Bmax ceiling compared to v38591 on IGROV-1 and TOV-21G, respectively. Negative control Palivizumab (v22277) showed no cellular binding, as expected.

TABLE 19.1
Cellular Binding of anti-NaPi2b Antibodies

| | IGROV-1 | | | TOV-21G | | |
|---|---|---|---|---|---|---|
| | Bmax | Kd | Curve Hill | | Kd | Curve Hill |
| Sample | (MFI) | (nM) | Slope (h) | Bmax | (nM) | Slope (h) |
| v38591 | 21,425 | 1.39 | 1.3 | 11,791 | 0.74 | 0.9 |
| v29456 | 20,096 | 1.28 | 1.1 | 11,271 | 0.60 | 1.1 |
| v18993 | 16,700 | 0.81 | 1.4 | 8,261 | 0.67 | 1.1 |
| (Lifastuzumab) | | | | | | |
| v22277 | NB | NB | NB | NB | NB | NB |
| (Palivizumab) | | | | | | |

* NB = no binding (apparent Kd value greater than the highest antibody testing concentration >150 nM)

### Example 20: Functional Characterization of Anti-NaPi2b Antibodies NaPi2b Specificity

The binding cross-reactivity of humanized antibody variant v38591 to human NaPi2b, NaPi2a, and NaPi2c was assessed by flow cytometry using HEK293-6e transfected cells. Reference anti-NaPi2b antibodies MX35 (v18992) and lifastuzumab (v18993) were included as positive controls; reference anti-NaPi2a (polyclonal rabbit anti-human SLC34A1; Atlas Biotechnologies Inc, Edmonton, AB; Cat. No. HPA051255) and anti-NaPi2c (polyclonal rabbit anti-human SLC34A3; Thermo Fisher Scientific, Waltham, MA; Cat. No. PA5-50762) antibodies were included; palivizumab (anti-RSV antibody, v22277) was included as a negative control.

HEK293-6e cells were maintained under standard culture conditions (37° C./5% CO2) with shaking at 110 rpm for suspension and cultured in FreeStyle™ 293 Expression Medium (Thermo Fisher Scientific, Waltham, MA) supplemented with 1% fetal bovine serum (Thermo Fisher Scientific, Waltham, MA) and 1× Penicillin-Streptomycin (Thermo Fisher Scientific, Waltham, MA). Cells were transfected with human NaPi2b (pTT5-huNaPi2b) (CL_#13432), human NaPi2a (CL_#13435), or human NaPi2c (CL_#13436) (all from GenScript Biotech, Piscataway, NJ), 1 μg of DNA per 1 million cells, using 293Fectin™ Transfection Reagent (Thermo Fisher Scientific, Waltham, MA) and Opti-MEM™ I Reduced Serum Medium (Thermo Fisher Scientific, Waltham, MA), and incubated for 24 hours under standard culture conditions (37° C./5% CO2/110 rpm). Following transfection, cells were seeded at 50,000 cells/well in conical-bottom 96-well plates and treated with test antibody for 24 hours at 4° C. to prevent internalization. Following incubation, cells were washed and stained with anti-Human IgG Fc AF647 conjugate (Jackson Immuno Research Labs, West Grove, PA; Cat. No. 109-605-098) or anti-Rabbit IgG F(ab″)2 AF647 conjugate (Jackson Immuno Research Labs, West Grove, PA; Cat. No. 111-605-047). Following incubation and washing, fluorescence was detected by flow cytometry on a BD LSRFortessa™ Cell Analyzer (BD Biosciences, Franklin Lake, NJ) with 1,000 minimum events collected per well. The AF647/APC-A GeoMean (fluorescence signal geometric mean, proportional to anti-Human AF647 binding) was calculated for the live cell population using FlowJo™ Version 10.8.1 (BD Biosciences, Franklin Lake, NJ) and plotted for each test antibody using GraphPad Prism Version 9 (GraphPad Software, San Diego, CA).

Results are shown in Table 20.1. Antibody variant v38591, and reference antibodies v18993 and v18992, displayed comparable binding to each other on HEK293-6e cells transfected with NaPi2b, and minimal binding to NaPi2a-transfected and NaPi2c-transfected HEK293-6e cells. Positive NaPi2a-binding control antibody from Atlas Bio demonstrated binding on NaPi2a-transfected cells, some binding to NaPi2c-transfected cells, and no binding to NaPi2b-transfected cells. Positive NaPi2c-binding control antibody from Thermo Fisher Scientific demonstrated some binding to NaPi2c-transfected cells, but not NaPi2a- or NaPi2b-transfected cells, as expected. Negative control Palivizumab (v22277) showed no cellular binding, as expected.

TABLE 20.1
Cross-reactivity of v38591 ADCs, parental antibody,
and controls to cynomolgus monkey and mouse NaPi2b

| | | Bmax (MFI) | | | Apparent Kd (nM) | | |
|---|---|---|---|---|---|---|---|
| | | Hu | Hu | Hu | Hu | Hu | Hu |
| Sample | DAR | NaPi2b | NaPi2a | NaPi2c | NaPi2b | NaPi2a | NaPi2c |
| v38591 | — | 651 | NB | NB | 1.68 | NB | NB |
| v18992 | — | 548 | NB | NB | 1.51 | NB | NB |
| (MX35) | | | | | | | |
| v18993 | — | 678 | NB | NB | 5.76 | NB | NB |
| (Lifastuzumab) | | | | | | | |
| v22277 | — | NB | NB | NB | NB | NB | NB |
| (Palivizumab) | | | | | | | |
| anti-SLC34A1 (Atlas | — | NB | IC | IC | NB | IC | IC |
| Biotechnologies Inc.) | | | | | | | |
| anti-SLC34A3 (Thermo | — | NB | NB | IC | NB | NB | IC |
| Fisher Scientific) | | | | | | | |

NB = no binding (apparent Kd value greater than the highest antibody testing concentration >200 nM for human antibodies, >50 nM for rabbit antibodies)
IC = incomplete curve

### Example 21: Pharmcokinetic Assessment of Anti-NaPi2b Antibodies in TG32 Mice

The pharmacokinetics (PK) of v29456 antibody and v18993 (lifastuzumab)-MCvcPABC-MMAE DAR4 were assessed in humanized FcRn Tg32 mice. This mouse model can be predictive of the pharmacokinetics of a drug in humans (see Avery et al. (2016) Utility of a human FcRn transgenic mouse model in drug discovery for early assessment and prediction of human pharmacokinetics of monoclonal antibodies, mAbs, 8:6, 1064-1078).

All test articles were administered at 5 mg/kg to hFcRn Tg32 mice (The Jackson Laboratory, Sacramento, CA; Stock #014565) by intravenous injection as shown in Table 26.1. For each test article, blood was collected from n=4 animals by retro-orbital bleed at 1, 3, and 6 hours and 1, 3, 7, 10, 14, 21 days post-dose. Blood was processed to serum and stored frozen at −80° C. in 96-well storage plates prior to pharmacokinetic analysis.

TABLE 26.1
Test articles and doses for PK assessment

| | | | Dose |
|---|---|---|---|
| Group | Test Article | DAR | (mg/kg) |
| 1 | v29456 | — | 5 |
| 7 | v18993 | 4 | 5 |
| | (lifastuzumab)- | | |
| | MCvcPABC-MMAE | | |

Mouse serum containing v29456 antibody and v18993 (lifastuzumab)-MCvcPABC-MMAE DAR4 was captured onto a 384 well plate coated with Goat anti-Human IgG Fc antibody (Jackson 109-005-098). The total antibody was detected with a goat anti-Human IgG Fab Biotin antibody (Jackson 109-065-097), followed by streptavidin SULFO-TAG conjugate (Mesoscale). After addition of MSD GOLD Read Buffer A, electrochemiluminescence (ECL) signal by the SULFO-TAG label was measured using the plate reader (Mesoscale).

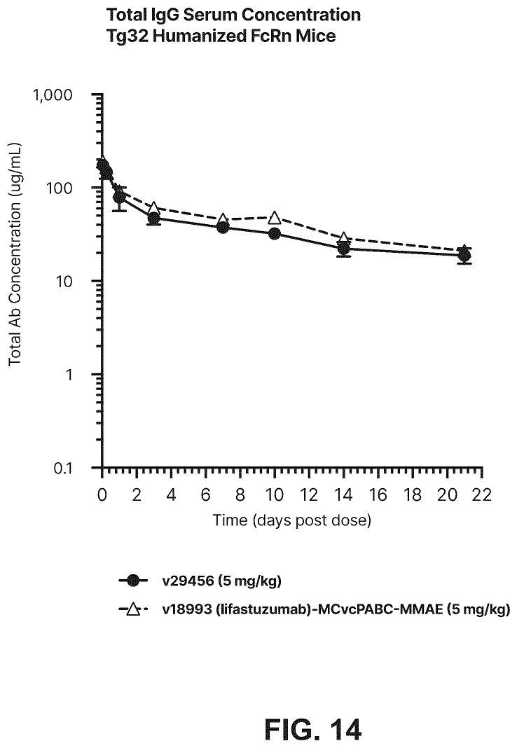

The PK profiles obtained are shown in FIG. 14. The anti-NaPi2b antibody v29456 demonstrated a typical antibody PK profile that was largely comparable between test articles and lifastuzumab-MMAE comparator.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

### Additional Sequence Tables (Seq Id Nos:36-59)

TABLE A
Clone Numbers for Variants

| | Clone Numbers | | | |
|---|---|---|---|---|
| Variant | H1 | H2 | L1 | L2 |
| 18993 | CL_#11677 | CL_#11678 | CL_#11695 | CL_#11695 |
| 18992 | CL_#11698 | CL_#11699 | CL_#11700 | CL_#11700 |
| 22277 | CL_#11009 | CL_#11010 | CL_#16635 | CL_#16635 |
| 21995 | CL_#16603 | CL_#16603 | CL_#16635 | CL_#16635 |
| 29449 | CL_#21506 | CL_#21506 | CL_#21509 | CL_#21509 |
| 29450 | CL_#21505 | CL_#21505 | CL_#21509 | CL_#21509 |
| 29451 | CL_#21504 | CL_#21504 | CL_#21509 | CL_#21509 |
| 29452 | CL_#21503 | CL_#21503 | CL_#21509 | CL_#21509 |
| 29453 | CL_#21506 | CL_#21506 | CL_#21508 | CL_#21508 |
| 29454 | CL_#21505 | CL_#21505 | CL_#21508 | CL_#21508 |
| 29455 | CL_#21504 | CL_#21504 | CL_#21508 | CL_#21508 |
| 29456 | CL_#21503 | CL_#21503 | CL_#21508 | CL_#21508 |
| 29457 | CL_#21506 | CL_#21506 | CL_#21507 | CL_#21507 |
| 29458 | CL_#21505 | CL_#21505 | CL_#21507 | CL_#21507 |
| 29459 | CL_#21504 | CL_#21504 | CL_#21507 | CL_#21507 |
| 29460 | CL_#21503 | CL_#21503 | CL_#21507 | CL_#21507 |
| 38591 | CL_#29371 | CL_#29371 | CL_#21508 | CL_#21508 |

TABLE B
Clone amino acid Sequences (Full Chain)

| Clone | | | SEQ ID |
|---|---|---|---|
| Number | Region | Sequence | NO |
| CL_#11009 | Full heavy | GQVTLRESGPALVKPTQTLTLTCTFSGFSLSTSGMSVGWIRQPPGKAL | 36 |
| | chain | EWLADIWWDDKKDYNPSLKSRLTISKDTSKNQVVLKVTNMDPADTA | |
| | | TYYCARSMITNWYFDVWGAGTTVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVYPPSRDELTKNQVSLTCLVKGF | |
| | | YPSDIAVEWESNGQPENNYKTTPPVLDSDGSFALVSKLTVDKSRWQQ | |
| | | GNVFSCSVMHEALHNHYTQKSLSLSPG | |
| CL_#11010 | Full heavy | GQVTLRESGPALVKPTQTLTLTCTFSGFSLSTSGMSVGWIRQPPGKAL | 37 |
| | chain | EWLADIWWDDKKDYNPSLKSRLTISKDTSKNQVVLKVTNMDPADTA | |
| | | TYYCARSMITNWYFDVWGAGTTVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVLPPSRDELTKNQVSLLCLVKGF | |
| | | YPSDIAVEWESNGQPENNYLTWPPVLDSDGSFFLYSKLTVDKSRWQQ | |
| | | GNVFSCSVMHEALHNHYTQKSLSLSPG | |
| CL_#11677 | Full heavy | GEVQLVESGGGLVQPGGSLRLSCAASGFSFSDFAMSWVRQAPGKGLE | 38 |
| | chain | WVATIGRVAFHTYYPDSMKGRFTISRDNSKNTLYLQMNSLRAEDTA | |
| | | VYYCARHRGFDVGHFDFWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVYPPSRDELTKNQVSLTCLVKGF | |
| | | YPSDIAVEWESNGQPENNYKTTPPVLDSDGSFALVSKLTVDKSRWQQ | |
| | | GNVFSCSVMHEALHNHYTQKSLSLSPG | |
| CL_#11678 | Full heavy | GEVQLVESGGGLVQPGGSLRLSCAASGFSFSDFAMSWVRQAPGKGLE | 39 |
| | chain | WVATIGRVAFHTYYPDSMKGRFTISRDNSKNTLYLQMNSLRAEDTA | |
| | | VYYCARHRGFDVGHFDFWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVLPPSRDELTKNQVSLLCLVKGF | |
| | | YPSDIAVEWESNGQPENNYLTWPPVLDSDGSFFLYSKLTVDKSRWQQ | |
| | | GNVFSCSVMHEALHNHYTQKSLSLSPG | |
| CL_#11695 | Full light | GDIQMTQSPSSLSASVGDRVTITCRSSETLVHSSGNTYLEWYQQKPGK | 40 |
| | chain | APKLLIYRVSNRFSGVPSRFSGSGSGTDFTLTISSLQPEDFATYYCFQGS | |
| | | FNPLTFGQGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |
| CL_#11698 | Full heavy | GQVQLVQSGAEVVKPGASVKMSCKASGYTFTGYNIHWVKQAPGQG | 41 |
| | chain | LEWIGAIYPGNGDTSYKQKFRGRATLTADTSTSTVYMELSSLRSEDSA | |
| | | VYYCARGETARATFAYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVYPPSRDELTKNQVSLTCLVKGF | |
| | | YPSDIAVEWESNGQPENNYKTTPPVLDSDGSFALVSKLTVDKSRWQQ | |
| | | GNVFSCSVMHEALHNHYTQKSLSLSPG | |
| CL_#11699 | Full heavy | GQVQLVQSGAEVVKPGASVKMSCKASGYTFTGYNIHWVKQAPGQG | 42 |
| | chain | LEWIGAIYPGNGDTSYKQKFRGRATLTADTSTSTVYMELSSLRSEDSA | |
| | | VYYCARGETARATFAYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVLPPSRDELTKNQVSLLCLVKGF | |
| | | YPSDIAVEWESNGQPENNYLTWPPVLDSDGSFFLYSKLTVDKSRWQQ | |
| | | GNVFSCSVMHEALHNHYTQKSLSLSPG | |
| CL_#11700 | Full light | GDIQMTQSPSSLSASVGDRVTITCSASQDIGNFLNWYQQKPGKTVKV | 43 |
| | chain | LIYYTSSLYSGVPSRFSGSGSGTDYTLTISSLQPEDFATYYCQQYSKLP | |
| | | LTFGQGTKLELKRTVAAPSVFIFPPSDEQLKSGTASVVCLLNNFYPRE | |
| | | AKVQWKVDNALQSGNSQESVTEQDSKDSTYSLSSTLTLSKADYEKH | |
| | | KVYACEVTHQGLSSPVTKSFNRGEC | |
| CL_#16603 | Full heavy | GQVTLRESGPALVKPTQTLTLTCTFSGFSLSTSGMSVGWIRQPPGKAL | 44 |
| | chain | EWLADIWWDDKKDYNPSLKSRLTISKDTSKNQVVLKVTNMDPADTA | |
| | | TYYCARSMITNWYFDVWGAGTTVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL_#16635 | Full light | GDIQMTQSPSTLSASVGDRVTITCKSQLSVGYMHWYQQKPGKAPKLL | 45 |
| | chain | IYDTSKLASGVPSRFSGSGSGTEFTLTISSLQPDDFATYYCFQGSGYPFT | |
| | | FGGGTKLEIKRTVAAPSVFIFPPSDEQLKSGTASVVCLLNNFYPREAK | |
| | | VQWKVDNALQSGNSQESVTEQDSKDSTYSLSSTLTLSKADYEKHKV | |
| | | YACEVTHQGLSSPVTKSFNRGEC | |
| CL_#21503 | Full heavy | QVQLVQSGAEVKKPGASVKVSCKASGFTFTSYNVHWVRQAPGQGLE | 46 |
| | chain | WMGAISPGNGDLSYAQKFQGRVTMTRDTSTSTVYMELSSLRSEDTA | |
| | | VYYCARGRTGMGAVDYWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL_#21504 | Full heavy | QVQLVQSGAEVKKPGASVKVSCKASGFTFTSYNVHWVRQAPGQGLE | 47 |
| | chain | WIAAISPGNGDLSYAQKFQGRVTMTVDTSTSTVYMELSSLRSEDTAV | |
| | | YYCARGRTGMGAVDYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL_#21505 | Full heavy | QVQLVQSGAEVKKPGASVKMSCKASGFTFTSYNVHWVRQAPGQGL | 48 |
| | chain | EWIAAISPGNGDLSYAQKFQGRATLTVDTSTSTAYMELSSLRSEDTAV | |
| | | YYCARGRTGMGAVDYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL #21506 | Full heavy | QVQLVQSGAEVKKPGASVKMSCKASGFTFTSYNVHWVRQAPGQGL | 49 |
| | chain | EWIAAISPGNGDLSYAQKFQGRATLTVDRSTSTAYMELSSLRSEDTAV | |
| | | YYCARGRTGMGAVDYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL #21507 | Full light | DIQMTQSPSSLSASVGDRVTITCRASQDVYSYLNWYQQKPGKAPKLLI | 50 |
| | chain | YYTSRLQSGVPSRFSGSGSGTDFTLTISSLQPEDFATYYCQQYNIFPWT | |
| | | FGQGTKLEIKRTVAAPSVFIFPPSDEQLKSGTASVVCLLNNFYPREAK | |
| | | VQWKVDNALQSGNSQESVTEQDSKDSTYSLSSTLTLSKADYEKHKV | |
| | | YACEVTHQGLSSPVTKSFNRGEC | |
| CL_#21508 | Full light | DVQMTQSPSSLSASVGDRVTITCRASQDVYSYLNWYQQKPGKAVKL | 51 |
| | chain | LIYYTSRLQSGVPSRFSGSGSGTDYTLTISSLQPEDFATYYCQQYNIFP | |
| | | WTFGQGTKLEIKRTVAAPSVFIFPPSDEQLKSGTASVVCLLNNFYPRE | |
| | | AKVQWKVDNALQSGNSQESVTEQDSKDSTYSLSSTLTLSKADYEKH | |
| | | KVYACEVTHQGLSSPVTKSFNRGEC | |
| CL_#21509 | Full light | DVQMTQSPSSLSASVGDRVTITCRASQDVYSYLNWYQQKPGKAVKL | 52 |
| | chain | LIYYTSRLQSGVPSRFSGSGSGTDYTLTISSLQPEDIATYYCQQYNIFP | |
| | | WTFGQGTKLEIKRTVAAPSVFIFPPSDEQLKSGTASVVCLLNNFYPRE | |
| | | AKVQWKVDNALQSGNSQESVTEQDSKDSTYSLSSTLTLSKADYEKH | |
| | | KVYACEVTHQGLSSPVTKSFNRGEC | |
| CL_#29371 | Full heavy | QVQLVQSGAEVKKPGASVKVSCKASGFTFTSYNVHWVRQAPGQGLE | 61 |
| | chain | WMGAISPGNGDLSYAQKFQGRVTMTRDTSTSTVYMELSSLRSEDTA | |
| | v38591 | VYYCARGRTGMGAVDYWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCL VKGF | |
| | | YPSDIAVEWESNGQPENNYKTTPPVLDSDGSFFLYSKLTVDKSRWQQ | |
| | | GNVFSCSVMHEALHNHYTQKSLSLSPGK | |

TABLE C
Clone DNA Sequences (VH, VL for v29449-29460)

| Clone | | | SEQ |
|---|---|---|---|
| Number | Region | Sequence | ID NO |
| CL_#21503 | VH | CAGGTGCAGCTGGTGCAGTCTGGAGCCGAGGTGAAGAAGCCAGGGG | 53 |
| | | CCAGCGTGAAGGTGTCCTGCAAGGCCTCTGGCTTCACCTTTACAAGC | |
| | | TACAACGTGCACTGGGTGAGGCAGGCCCCCGGACAGGGACTGGAGT | |
| | | GGATGGGAGCCATCAGCCCTGGCAATGGCGACCTGTCCTACGCCCA | |
| | | GAAGTTTCAGGGCAGGGTGACCATGACACGCGATACCTCCACATCT | |
| | | ACCGTGTATATGGAGCTGAGCTCCCTGAGATCCGAGGACACAGCCG | |
| | | TGTACTATTGTGCCCGGGGCAGAACCGGAATGGGAGCCGTGGATTA | |
| | | TTGGGGCCAGGGCACACTGGTGACCGTGTCTAGC | |
| CL_#21504 | VH | CAGGTGCAGCTGGTGCAGTCTGGAGCCGAGGTGAAGAAGCCAGGGG | 54 |
| | | CCAGCGTGAAGGTGTCCTGCAAGGCCTCTGGCTTCACCTTTACAAGC | |
| | | TACAACGTGCACTGGGTGCGGCAGGCCCCCGGACAGGGACTGGAGT | |
| | | GGATCGCAGCAATCAGCCCTGGCAATGGCGACCTGTCCTACGCCCA | |
| | | GAAGTTTCAGGGCCGGGTGACCATGACAGTGGATACCTCCACATCTA | |
| | | CCGTGTATATGGAGCTGAGCTCCCTGAGGTCCGAGGACACAGCCGT | |
| | | GTACTATTGTGCCCGGGGCAGAACCGGAATGGGAGCCGTGGATTAT | |
| | | TGGGGCCAGGGCACACTGGTGACCGTGTCTAGC | |
| CL_#21505 | VH | CAGGTGCAGCTGGTGCAGTCTGGAGCCGAGGTGAAGAAGCCAGGGG | 55 |
| | | CCAGCGTGAAGATGTCCTGCAAGGCCTCTGGCTTCACCTTTACAAGC | |
| | | TACAACGTGCACTGGGTGCGGCAGGCCCCCGGACAGGGACTGGAGT | |
| | | GGATCGCAGCAATCAGCCCTGGCAATGGCGACCTGTCCTACGCCCA | |
| | | GAAGTTTCAGGGCCGGGCCACCCTGACAGTGGATACCTCCACATCTA | |
| | | CCGCCTATATGGAGCTGAGCTCCCTGAGGTCCGAGGACACAGCCGT | |
| | | GTACTATTGTGCCCGGGGCAGAACCGGAATGGGAGCCGTGGATTAT | |
| | | TGGGGCCAGGGCACACTGGTGACCGTGTCTAGC | |
| CL_#21506 | VH | CAGGTGCAGCTGGTGCAGTCTGGAGCCGAGGTGAAGAAGCCAGGGG | 56 |
| | | CCAGCGTGAAGATGTCCTGCAAGGCCTCTGGCTTCACCTTTACAAGC | |
| | | TACAACGTGCACTGGGTGAGGCAGGCCCCCGGACAGGGACTGGAGT | |
| | | GGATCGCAGCAATCAGCCCTGGCAATGGCGACCTGTCCTACGCCCA | |
| | | GAAGTTTCAGGGCAGGGCCACCCTGACAGTGGATCGCTCCACCTCTA | |
| | | CAGCCTATATGGAGCTGAGCTCCCTGAGATCCGAGGACACCGCCGT | |
| | | GTACTATTGTGCCCGGGGCAGAACAGGAATGGGAGCCGTGGATTAT | |
| | | TGGGGCCAGGGCACCCTGGTGACAGTGTCTAGC | |
| CL_#21507 | VL | GACATCCAGATGACACAGTCTCCAAGCTCCCTGTCCGCCTCTGTGGG | 57 |
| | | CGACAGGGTGACCATCACATGCAGAGCCAGCCAGGACGTGTACTCC | |
| | | TATCTGAACTGGTACCAGCAGAAGCCCGGCAAGGCCCCTAAGCTGC | |
| | | TGATCTACTATACCAGCAGGCTGCAGTCCGGAGTGCCTTCTCGCTTC | |
| | | AGCGGCTCCGGCTCTGGAACCGACTTTACCCTGACAATCTCTAGCCT | |
| | | GCAGCCTGAGGATTTTGCCACATACTATTGTCAGCAGTATAATATCT | |
| | | TCCCATGGACCTTTGGCCAGGGCACAAAGCTGGAGATCAAG | |
| CL_#21508 | VL | GACGTGCAGATGACACAGTCTCCTAGCTCCCTGTCCGCCTCTGTGGG | 58 |
| | | CGACAGGGTGACCATCACATGCAGAGCCAGCCAGGACGTGTACTCC | |
| | | TATCTGAACTGGTACCAGCAGAAGCCTGGCAAGGCCGTGAAGCTGC | |
| | | TGATCTACTATACCAGCAGGCTGCAGTCCGGAGTGCCATCTCGCTTC | |
| | | AGCGGCTCCGGCTCTGGAACCGACTACACCCTGACAATCTCTAGCCT | |
| | | GCAGCCAGAGGATTTTGCCACATACTATTGTCAGCAGTATAATATCT | |
| | | TCCCCTGGACCTTTGGCCAGGGCACAAAGCTGGAGATCAAG | |
| CL_#21509 | VL | GACGTGCAGATGACACAGTCTCCTAGCTCCCTGTCCGCCTCTGTGGG | 59 |
| | | CGACAGGGTGACCATCACATGCAGAGCCAGCCAGGACGTGTACTCC | |
| | | TATCTGAACTGGTACCAGCAGAAGCCTGGCAAGGCCGTGAAGCTGC | |
| | | TGATCTACTATACCAGCAGGCTGCAGTCCGGCGTGCCATCTCGCTTT | |
| | | AGCGGCTCCGGCTCTGGAACCGACTACACCCTGACAATCTCTAGCCT | |
| | | GCAGCCAGAGGATATCGCCACATACTATTGTCAGCAGTATAATATCT | |
| | | TCCCCTGGACCTTTGGCCAGGGCACAAAGCTGGAGATCAAG | |

1. An antibody construct comprising an antigen-binding domain that binds to human NaPi2b (sodium-dependent phosphate transport protein 2B), the antigen-binding domain comprising:
a) a heavy chain CDR1 (HCDR1) amino acid sequence comprising the sequence as set forth in SEQ ID NO: 7, a heavy chain CDR2 (HCDR2) amino acid sequence comprising the sequence as set forth in SEQ ID NO: 8, and a heavy chain CDR3 (HCDR3) amino acid sequence comprising the sequence as set forth in SEQ ID NO: 9, and
b) a light chain CDR1 (LCDR1) amino acid sequence comprising the sequence as set forth in SEQ ID NO: 19, a light chain CDR2 (LCDR2) amino acid sequence comprising the sequence as set forth in SEQ ID NO: 20, and a light chain CDR3 (LCDR3) amino acid sequence comprising the sequence as set forth in SEQ ID NO: 18.

2. The antibody-drug construct according to claim 1, wherein the antigen-binding domain comprises:
a) a VH domain having at least 90% sequence identity to the sequence as set forth in SEQ ID NO: 24 and a VL domain having at least 90% sequence identity to the sequence as set forth in SEQ ID NO: 29;
b) a VH domain having at least 90% sequence identity to the sequence as set forth in SEQ ID NO: 24 and a VL domain having at least 90% sequence identity to the sequence as set forth in SEQ ID NO: 30;
c) a VH domain having at least 90% sequence identity to the sequence as set forth in SEQ ID NO: 26 and a VL domain having at least 90% sequence identity to the sequence as set forth in SEQ ID NO: 30;
d) a VH domain having at least 90% sequence identity to the sequence as set forth in SEQ ID NO: 25 and a VL domain having at least 90% sequence identity to the sequence as set forth in SEQ ID NO: 30;
e) a VH domain having at least 90% sequence identity to the sequence as set forth in SEQ ID NO: 27 and a VL domain having at least 90% sequence identity to the sequence as set forth in SEQ ID NO: 30;
f) a VH domain having at least 90% sequence identity to the sequence as set forth in SEQ ID NO: 27 and a VL domain having at least 90% sequence identity to the sequence as set forth in SEQ ID NO: 29;
g) a VH domain having at least 90% sequence identity to the sequence as set forth in SEQ ID NO: 26 and a VL domain having at least 90% sequence identity to the sequence as set forth in SEQ ID NO: 29;
h) a VH domain having at least 90% sequence identity to the sequence as set forth in SEQ ID NO: 25 and a VL domain having at least 90% sequence identity to the sequence as set forth in SEQ ID NO: 29;
i) a VH domain having at least 90% sequence identity to the sequence as set forth in SEQ ID NO: 27 and a VL domain having at least 90% sequence identity to the sequence as set forth in SEQ ID NO: 28;
j) a VH domain having at least 90% sequence identity to the sequence as set forth in SEQ ID NO: 26 and a VL domain having at least 90% sequence identity to the sequence as set forth in SEQ ID NO: 28;
k) a VH domain having at least 90% sequence identity to the sequence as set forth in SEQ ID NO: 25 and a VL domain having at least 90% sequence identity to the sequence as set forth in SEQ ID NO: 28;
l) a VH domain having at least 90% sequence identity to the sequence as set forth in SEQ ID NO: 24 and a VL domain having at least 90% sequence identity to the sequence as set forth in SEQ ID NO: 28; or
m) a VH domain having at least 90% sequence identity to the sequence as set forth in SEQ ID NO: 31 and a VL domain having at least 90% sequence identity to the sequence as set forth in SEQ ID NO: 32.

3. The antibody construct according to claim 1, further comprising a scaffold, wherein the antigen-binding domain is operably linked to the scaffold.

4. The antibody construct according to claim 3, wherein the scaffold comprises an IgG Fc region.

5. The antibody construct according to claim 1, wherein the antibody construct further comprises a second antigen-binding domain.

6. The antibody construct according to claim 5, wherein the second antigen-binding domain binds to NaPi2b.

7. The antibody construct according to claim 6, wherein the second antigen-binding domain is the same as the first antigen-binding domain.

8. The antibody construct according to claim 5, further comprising one or more additional antigen-binding domains.

9. A polynucleotide or set of polynucleotides encoding the antibody construct according to claim 1.

10. An expression vector or set of expression vectors comprising the polynucleotide or set of polynucleotides according to claim 9.

11. A host cell comprising the expression vector or set of expression vectors according to claim 10.

12. An antibody-drug conjugate comprising the antibody construct according to claim 1 conjugated to one or more drug moieties.

13. The antibody-drug conjugate according to claim 12, wherein the antibody conjugate is conjugated to between 1 and about 8 drug moieties.

14. The antibody-drug conjugate according to claim 13, wherein the antibody-drug conjugate has general Formula I:
A-(L-(D)ₘ)ₙ (I)
wherein:
A is the antibody construct;
L is a linker;
D is a drug moiety;
m is an integer between 1 and about 8, and
n is between 1 and about 12.

15. The antibody-drug conjugate according to claim 14, wherein m is 1 or 2.

16. The antibody-drug conjugate according to claim 14, wherein n is between about 2 and about 8.

17. The antibody-drug conjugate according to claim 13, wherein the drug moiety is a maytansinoid, maytansinoid analogue, benzodiazepine, pyrrolobenzodiazepine, duocarmycin, calicheamicin, calicheamicin analogue, auristatin, auristatin analogue, hemiasterlin, hemiasterlin analogue, tubulysin, tubulysin analogue, amatoxin, amatoxin analogue, camptothecin, camptothecin analogue, eribulin, TLR agonist or STING agonist.

18. The antibody-drug conjugate according to claim 13, wherein the drug moiety an auristatin, auristatin analogue, hemiasterlin, hemiasterlin analogue, camptothecin, camptothecin analogue or eribulin.

19. A pharmaceutical composition comprising the antibody construct according to claim 1, and a pharmaceutically acceptable carrier or diluent.

20.-22. (canceled)

23. A method of inhibiting the growth of NaPi2b-positive tumor cells comprising contacting the cells with the antibody construct according to claim 1.

24. A method of treating a subject having a cancer comprising administering to the subject an effective amount of the antibody construct according to claim 1.

25. A pharmaceutical composition comprising the antibody-drug conjugate according to claim 12 and a pharmaceutically acceptable carrier or diluent.

26. A method of inhibiting the growth of NaPi2b-positive tumor cells comprising contacting the cells with the antibody-drug conjugate according to claim 12.

27. A method of treating a subject having a cancer comprising administering to the subject an effective amount of the antibody-drug conjugate according to claim 12.


SEQUENCE LISTING (61 sequences)

SEQ ID NO: 1 - protein, 690 aa, Homo sapiens
  source 1..690: note = NaPi2b
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCCCCRVCCRACCLLCDCPKCCRCSKCCEDLEEAQEGQDVPVKAPETFDNITISREAQGEVPASDSKTECTAL

SEQ ID NO: 2 - protein, 690 aa, Macaca fascicularis
  source 1..690: note = NaPi2b
MAPWPELGDAQPNPSKYLEGATSQQPITPDKSKETNKTDNTEVPVTKFELLPTYSTATLIEEPTEVDDPWNLPTLQDSGIKWSERDAKGKILCVFQGIGRLILLLGFLYFFVCSLDVLSSAFQLVGGKMAGQFFSNSSIMSNPLLGLVIGVLVTVFVQSSSTSTSIVVSMVASSLLTVRAAIPIIMGANIGTSITNTIVALMQVGDRSEFRRAFAGATVHDFFNWLSVLVLLPVEVATHYLEVVTQLIVESFHFKNGEDAPDLLKVITKPFTKLIVQLDKKVISQIAMNDETAKNKSLVKIWCKTFTNMTQMNVTVPSMANCTSHSLCWTDGIQIWTMKNVTYKENIAKCQHIFVNFHLPDLAVGIILLIISLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNTLRSSLQIALCHFFFNISGILLWYPIPFTRLPIRMAKGLGNISAKYRWFAVFYLIIFFFLIPLTVFGLSLAGWPVLVGVGVPVVFIIILVLCLRLLQSRCPRVLPKKLQNWNFLPLCMHSLKPWDAVISKFTGPFQMPCCCCCRVCCRVCCLLCGCPKCCRCSKCCQDLEEGQEAQGVPVKAPETFDNITISREAQGEVRAPDSKTECTAL

SEQ ID NO: 3 - protein, 697 aa, Mus sp.
  source 1..697: note = NaPi2b
MAPWPELENAQPNPGKFIEGASGPQSSIPAKDKEASKTNDNGTPVAKTELLPSYSALVLIEEHPEGTDPWDLPELQDTGIKWSERDTKGKTLCIFQGVGKFILLLGFLYLFVCSLDVLSSAFQLVGGKVAGQFFSNNSIMSNPVAGLVIGVLVTVMVQSSSTSSSIIVSMVASSLLTVRAAIPIIMGANIGTSITNTIVALMQAGDRNEFRRAFAGATVHDFFNWLSVFVLLPLEAATHYLEILTNLVLETFKFQNGEDAPDILKVITDPFTKLIIQLDKKVIQQIAMGDSAAQNKSLIKIWCKSITNVTEMNVTVPSTDNCTSPSYCWTDGIQTWTIQNVTQKENIAKCQHIFVNFSLPDLAVGIILLTVSLVVLCGCLIMIVKLLGSVLRGQVATVIKKTLNTDFPFPFAWLTGYLAILVGAGMTFIVQSSSVFTSAMTPLIGIGVISIERAYPLTLGSNIGTTTTAILAALASPGNTLRSSLQIALCHFFFNISGILLWYPIPFTRLPIRLAKGLGNISAKYRWFAVFYLIFFFFVTPLTVFGLSLAGWPVLVGVGVPIILLLLLVLCLRMLQFRCPRILPLKLRDWNFLPLWMHSLKPWDNVISLATTCFQRRCCCCCRVCCRVCCMVCGCKCCRCSKCCRDQGEEEEEKEQDIPVKASGAFDNAAMSKECQDEGKGQVEVLSMKALSNTTVF

SEQ ID NO: 4 - protein, 8 aa, synthetic construct
GFTFTSYN

SEQ ID NO: 5 - protein, 8 aa, synthetic construct
  source 1..8: note = IMGT HCDR2
ISPGNGDL

SEQ ID NO: 6 - protein, 12 aa, synthetic construct
  source 1..12: note = IMGT HCDR3
ARGRTGMGAVDY

SEQ ID NO: 7 - protein, 5 aa, synthetic construct
  source 1..5: note = Kabat HCDR1
SYNVH

SEQ ID NO: 8 - protein, 17 aa, synthetic construct
  source 1..17: note = Kabat HCDR2
AISPGNGDLSYNQRFKD

SEQ ID NO: 9 - protein, 10 aa, synthetic construct
  source 1..10: note = Kabat, Chothia, AbM HCDR3
GRTGMGAVDY

SEQ ID NO: 10 - protein, 7 aa, synthetic construct
  source 1..7: note = Chothia HCDR1
GFTFTSY

SEQ ID NO: 11 - protein, 6 aa, synthetic construct
  source 1..6: note = Chothia HCDR2
SPGNGD

SEQ ID NO: 12 - protein, 10 aa, synthetic construct
  source 1..10: note = AbM HCDR1
GFTFTSYNVH

SEQ ID NO: 13 - protein, 10 aa, synthetic construct
  source 1..10: note = AbM HCDR2
AISPGNGDLS

SEQ ID NO: 14 - protein, 6 aa, synthetic construct
  source 1..6: note = Contact HCDR1
TSYNVH

SEQ ID NO: 15 - protein, 13 aa, synthetic construct
  source 1..13: note = Contact HCDR2
WIAAISPGNGDLS

SEQ ID NO: 16 - protein, 11 aa, synthetic construct
  source 1..11: note = Contact HCDR3
ARGRTGMGAVD

SEQ ID NO: 17 - protein, 6 aa, synthetic construct
  source 1..6: note = IMGT LCDR1
QDVYSY

SEQ ID NO: 18 - protein, 9 aa, synthetic construct
  source 1..9: note = IMGT, Kabat, Chothia, AbM LCDR3
QQYNIFPWT

SEQ ID NO: 19 - protein, 11 aa, synthetic construct
  source 1..11: note = Kabat, Chothia, AbM LCDR1
RASQDVYSYLN

SEQ ID NO: 20 - protein, 7 aa, synthetic construct
  source 1..7: note = Kabat, Chothia, AbM LCDR2
YTSRLQS

SEQ ID NO: 21 - protein, 7 aa, synthetic construct
  source 1..7: note = Contact LCDR1
YSYLNWY

SEQ ID NO: 22 - protein, 10 aa, synthetic construct
  source 1..10: note = Contact LCDR2
LLIYYTSRLQ

SEQ ID NO: 23 - protein, 8 aa, synthetic construct
  source 1..8: note = Contact LCDR3
QQYNIFPW

SEQ ID NO: 24 - protein, 119 aa, synthetic construct
  source 1..119: note = v29452, v29456, v29460 VH (H1)
QVQLVQSGAEVKKPGASVKVSCKASGFTFTSYNVHWVRQAPGQGLEWMGAISPGNGDLSYAQKFQGRVTMTRDTSTSTVYMELSSLRSEDTAVYYCARGRTGMGAVDYWGQGTLVTVSS

SEQ ID NO: 25 - protein, 119 aa, synthetic construct
  source 1..119: note = v29451, v29455, v29459 VH (H2)
QVQLVQSGAEVKKPGASVKVSCKASGFTFTSYNVHWVRQAPGQGLEWIAAISPGNGDLSYAQKFQGRVTMTVDTSTSTVYMELSSLRSEDTAVYYCARGRTGMGAVDYWGQGTLVTVSS

SEQ ID NO: 26 - protein, 119 aa, synthetic construct
  source 1..119: note = v29450, v29454, v29458 VH (H3)
QVQLVQSGAEVKKPGASVKMSCKASGFTFTSYNVHWVRQAPGQGLEWIAAISPGNGDLSYAQKFQGRATLTVDTSTSTAYMELSSLRSEDTAVYYCARGRTGMGAVDYWGQGTLVTVSS

SEQ ID NO: 27 - protein, 119 aa, synthetic construct
  source 1..119: note = v29449, v29453, v29457 VH (H4)
QVQLVQSGAEVKKPGASVKMSCKASGFTFTSYNVHWVRQAPGQGLEWIAAISPGNGDLSYAQKFQGRATLTVDRSTSTAYMELSSLRSEDTAVYYCARGRTGMGAVDYWGQGTLVTVSS

SEQ ID NO: 28 - protein, 107 aa, synthetic construct
  source 1..107: note = v29457, v29458, v29459, v29460 VL (L1)
DIQMTQSPSSLSASVGDRVTITCRASQDVYSYLNWYQQKPGKAPKLLIYYTSRLQSGVPSRFSGSGSGTDFTLTISSLQPEDFATYYCQQYNIFPWTFGQGTKLEIK

SEQ ID NO: 29 - protein, 107 aa, synthetic construct
  source 1..107: note = v29453, v29454, v29455, v29456 VL (L2)
DVQMTQSPSSLSASVGDRVTITCRASQDVYSYLNWYQQKPGKAVKLLIYYTSRLQSGVPSRFSGSGSGTDYTLTISSLQPEDFATYYCQQYNIFPWTFGQGTKLEIK

SEQ ID NO: 30 - protein, 107 aa, synthetic construct
  source 1..107: note = v29449, v29450, v29451, v29452 VL (L3)
DVQMTQSPSSLSASVGDRVTITCRASQDVYSYLNWYQQKPGKAVKLLIYYTSRLQSGVPSRFSGSGSGTDYTLTISSLQPEDIATYYCQQYNIFPWTFGQGTKLEIK

SEQ ID NO: 31 - protein, 119 aa, synthetic construct
  source 1..119: note = v23855 VH
QAYLQQSGAELVRPGASVKMSCKASGFTFTSYNVHWVKQTPRQGLEWIAAISPGNGDLSYNQRFKDKATLTVDRSSSTAYMQLSGLTSEDSAVYFCARGRTGMGAVDYWGQGTSVTVSS

SEQ ID NO: 32 - protein, 107 aa, synthetic construct
  source 1..107: note = v23855 VL
DVQMTQTASSLSASLGDRVTISCRASQDVYSYLNWYQQKPHGTVKLLIYYTSRLQSGVPSRFTGSGSGTDYSLTISNLEPEDIATYYCQQYNIFPWTFGGGTKLEIK

SEQ ID NO: 33 - protein, 329 aa, synthetic construct
  source 1..329: note = CH1-hinge-CH2-CH3 (IGHG1*01)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 107 aa, synthetic construct [sequence omitted]

SEQ ID NO: 35 - protein, 23 aa, synthetic construct
  source 1..23: note = signal peptide
MRPTWAWWLFLVLLLALWAPARG

SEQ ID NO: 36 - protein, 450 aa, synthetic construct
  source 1..450: note = CL_#11009 Full Heavy Chain
GQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 37 - protein, 450 aa, synthetic construct
  source 1..450: note = CL_#11010 Full Heavy Chain
GQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 38 - protein, 450 aa, synthetic construct
  source 1..450: note = CL_#11677 Full Heavy Chain
GEVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 39 - protein, 450 aa, synthetic construct
  source 1..450: note = CL_#11678 Full Heavy Chain
GEVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 40 - protein, 220 aa, synthetic construct
  source 1..220: note = CL_#11695 Full Light Chain
GDIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 449 aa, synthetic construct
  source 1..449: note = CL_#11698 Full Heavy Chain
GQVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 42 - protein, 449 aa, synthetic construct
  source 1..449: note = CL_#11699 Full Heavy Chain
GQVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 43 - protein, 215 aa, synthetic construct
  source 1..215: note = CL_#11700 Full Light Chain
GDIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 450 aa, synthetic construct
  source 1..450: note = CL_#16603 Full Heavy Chain
GQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 214 aa, synthetic construct
  source 1..214: note = CL_#16635 Full Light Chain
GDIQMTQSPSTLSASVGDRVTITCKSQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 448 aa, synthetic construct
  source 1..448: note = CL_#21503 Full Heavy Chain
QVQLVQSGAEVKKPGASVKVSCKASGFTFTSYNVHWVRQAPGQGLEWMGAISPGNGDLSYAQKFQGRVTMTRDTSTSTVYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 448 aa, synthetic construct
  source 1..448: note = CL_#21504 Full Heavy Chain
QVQLVQSGAEVKKPGASVKVSCKASGFTFTSYNVHWVRQAPGQGLEWIAAISPGNGDLSYAQKFQGRVTMTVDTSTSTVYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 448 aa, synthetic construct
  source 1..448: note = CL_#21505 Full Heavy Chain
QVQLVQSGAEVKKPGASVKMSCKASGFTFTSYNVHWVRQAPGQGLEWIAAISPGNGDLSYAQKFQGRATLTVDTSTSTAYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 448 aa, synthetic construct
  source 1..448: note = CL_#21506 Full Heavy Chain
QVQLVQSGAEVKKPGASVKMSCKASGFTFTSYNVHWVRQAPGQGLEWIAAISPGNGDLSYAQKFQGRATLTVDRSTSTAYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 214 aa, synthetic construct
  source 1..214: note = CL_#21507 Full Light Chain
DIQMTQSPSSLSASVGDRVTITCRASQDVYSYLNWYQQKPGKAPKLLIYYTSRLQSGVPSRFSGSGSGTDFTLTISSLQPEDFATYYCQQYNIFPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, synthetic construct
  source 1..214: note = CL_#21508 Full Light Chain
DVQMTQSPSSLSASVGDRVTITCRASQDVYSYLNWYQQKPGKAVKLLIYYTSRLQSGVPSRFSGSGSGTDYTLTISSLQPEDFATYYCQQYNIFPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 214 aa, synthetic construct
  source 1..214: note = CL_#21509 Full Light Chain
DVQMTQSPSSLSASVGDRVTITCRASQDVYSYLNWYQQKPGKAVKLLIYYTSRLQSGVPSRFSGSGSGTDYTLTISSLQPEDIATYYCQQYNIFPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 357 bp, synthetic construct
  source 1..357: mol_type = other DNA; note = CL_#21503 VH
caggtgcagctggtgcagtctggagccgaggtgaagaagccaggggccagcgtgaaggtgtcctgcaaggcctctggcttcacctttacaagctacaacgtgcactgggtgaggcaggcccccggacagggactggagtggatgggagccatcagccctggcaatggcgacctgtcctacgcccagaagtttcagggcagggtgaccatgacacgcgatacctccacatctaccgtgtatatggagctgagctccctgagatccgaggacacagccgtgtactattgtgcccggggcagaaccggaatgggagccgtggattattggggccagggcacactggtgaccgtgtctagc

SEQ ID NO: 54 - DNA, 357 bp, synthetic construct
  source 1..357: mol_type = other DNA; note = CL_#21504 VH
caggtgcagctggtgcagtctggagccgaggtgaagaagccaggggccagcgtgaaggtgtcctgcaaggcctctggcttcacctttacaagctacaacgtgcactgggtgcggcaggcccccggacagggactggagtggatcgcagcaatcagccctggcaatggcgacctgtcctacgcccagaagtttcagggccgggtgaccatgacagtggatacctccacatctaccgtgtatatggagctgagctccctgaggtccgaggacacagccgtgtactattgtgcccggggcagaaccggaatgggagccgtggattattggggccagggcacactggtgaccgtgtctagc

SEQ ID NO: 55 - DNA, 357 bp, synthetic construct
  source 1..357: mol_type = other DNA; note = CL_#21505 VH
caggtgcagctggtgcagtctggagccgaggtgaagaagccaggggccagcgtgaagatgtcctgcaaggcctctggcttcacctttacaagctacaacgtgcactgggtgcggcaggcccccggacagggactggagtggatcgcagcaatcagccctggcaatggcgacctgtcctacgcccagaagtttcagggccgggccaccctgacagtggatacctccacatctaccgcctatatggagctgagctccctgaggtccgaggacacagccgtgtactattgtgcccggggcagaaccggaatgggagccgtggattattggggccagggcacactggtgaccgtgtctagc

SEQ ID NO: 56 - DNA, 357 bp, synthetic construct
  source 1..357: mol_type = other DNA; note = CL_#21506 VH
caggtgcagctggtgcagtctggagccgaggtgaagaagccaggggccagcgtgaagatgtcctgcaaggcctctggcttcacctttacaagctacaacgtgcactgggtgaggcaggcccccggacagggactggagtggatcgcagcaatcagccctggcaatggcgacctgtcctacgcccagaagtttcagggcagggccaccctgacagtggatcgctccacctctacagcctatatggagctgagctccctgagatccgaggacaccgccgtgtactattgtgcccggggcagaacaggaatgggagccgtggattattggggccagggcaccctggtgacagtgtctagc

SEQ ID NO: 57 - DNA, 321 bp, synthetic construct
  source 1..321: mol_type = other DNA; note = CL_#21507 VL
gacatccagatgacacagtctccaagctccctgtccgcctctgtgggcgacagggtgaccatcacatgcagagccagccaggacgtgtactcctatctgaactggtaccagcagaagcccggcaaggcccctaagctgctgatctactataccagcaggctgcagtccggagtgccttctcgcttcagcggctccggctctggaaccgactttaccctgacaatctctagcctgcagcctgaggattttgccacatactattgtcagcagtataatatcttcccatggacctttggccagggcacaaagctggagatcaag

SEQ ID NO: 58 - DNA, 321 bp, synthetic construct
  source 1..321: mol_type = other DNA; note = CL_#21508 VL
gacgtgcagatgacacagtctcctagctccctgtccgcctctgtgggcgacagggtgaccatcacatgcagagccagccaggacgtgtactcctatctgaactggtaccagcagaagcctggcaaggccgtgaagctgctgatctactataccagcaggctgcagtccggagtgccatctcgcttcagcggctccggctctggaaccgactacaccctgacaatctctagcctgcagccagaggattttgccacatactattgtcagcagtataatatcttcccctggacctttggccagggcacaaagctggagatcaag

SEQ ID NO: 59 - DNA, 321 bp, synthetic construct
  source 1..321: mol_type = other DNA; note = CL_#21509 VL
gacgtgcagatgacacagtctcctagctccctgtccgcctctgtgggcgacagggtgaccatcacatgcagagccagccaggacgtgtactcctatctgaactggtaccagcagaagcctggcaaggccgtgaagctgctgatctactataccagcaggctgcagtccggcgtgccatctcgctttagcggctccggctctggaaccgactacaccctgacaatctctagcctgcagccagaggatatcgccacatactattgtcagcagtataatatcttcccctggacctttggccagggcacaaagctggagatcaag

SEQ ID NO: 60 - protein, 217 aa, synthetic construct
  source 1..217: note = IgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 449 aa, synthetic construct
  source 1..449: note = CL_#29371 Full Heavy Chain
QVQLVQSGAEVKKPGASVKVSCKASGFTFTSYNVHWVRQAPGQGLEWMGAISPGNGDLSYAQKFQGRVTMTRDTSTSTVYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK